(19) United States
(12) Patent Application Publication
(10) Pub. No.: US 2016/0130361 A1
(43) Pub. Date: May 12, 2016

(54) ANTI-FACTOR VIII ANTIBODIES OR USES THEREOF

(71) Applicant: BIOGEN MA INC., Cambridge, MA (US)

(72) Inventor: John KULMAN, Belmont, MA (US)

(73) Assignee: Biogen MA Inc., Cambridge, MA (US)

(21) Appl. No.: 14/894,155

(22) PCT Filed: Jun. 13, 2014
(86) PCT No.: PCT/US2014/042374

§ 371 Date: Nov. 25, 2015

Related U.S. Application Data
(60) Provisional application No. 61/834,828, filed on Jun. 13, 2013.
(60) Provisional application No. 61/913,143, filed on Dec. 6, 2013.

(51) Int. Cl. C07K 16/36; G01N 33/86; C07K 14/755

(52) U.S. Cl. CPC C07K 16/36; C07K 14/755; G01N 33/86; C07K 2317/565; C07K 2317/56; C07K 2317/92; C07K 2317/76; C07K 2317/55; C07K 2317/52; G01N 2333/755; G01N 2800/224; A61K 38/00

(57) ABSTRACT

The present invention provides anti-FVIII antibodies and antigen-binding molecules thereof which specifically bind FVIII epitopes. The antibodies and antigen-binding molecules can be used to purify FVIII. The invention also includes nucleic acid molecules and methods of making the anti-FVIII antibodies or antigen binding molecules thereof.

### BACKGROUND OF THE INVENTION

Hemophilia A is characterized by spontaneous hemorrhage and excessive bleeding after trauma. Treatment of hemophilia A is by replacement therapy targeting restoration of Factor VIII (“FVIII”) activity. Treatment of hemophilia A is by replacement therapy targeting restoration of FVIII activity to 1 to 5% of normal levels to prevent spontaneous bleeding (Mannucci, P. M., et al., N. Engl. J. Med. 344:1773-1779 (2001), which is herein incorporated by reference in its entirety). There are plasma-derived and recombinant FVIII products available to treat bleeding episodes on-demand or to prevent bleeding episodes from occurring by treating prophylactically.

About 25% of hemophilia A patients under replacement therapy by FVIII infusion develop an immune response against FVIII. Anti-FVIII antibodies can also be found in the context of some autoimmune diseases, or occasionally after pregnancy or surgery. Such antibodies, called inhibitors, reduce the rate of thrombin generation by the tenase complex and thereby inhibit the amplification loop of the coagulation cascade.

Plasma-derived FVIII or recombinantly-produced FVIII can be purified by anti-FVIII antibodies or anti-Von Willebrand factor (VWF) antibodies. However, purification of FVIII products can be challenging because of FVIII's instability and low product yields. For example, FVIII can be unstable and can be susceptible to dissociation under certain acidic conditions due to a metal ion bridge connecting FVIII's heavy chain with the light chain. In addition to the instability, purification of FVIII products typically requires four or five chromatography steps to achieve acceptable levels of product purity and product potency. Moreover, the multiple steps of chromatography can reduce product yields. While some anti-FVIII antibodies bind to FVIII and thus are candidates for immunoaffinity purification, the anti-FVIII antibodies may affect the coagulation activity of the FVIII product after the FVIII product is released from the antibody. Moreover, improved immunoaffinity purification methods employing novel antibodies would be useful for purifying modified variants of FVIII for which conventional chromatographic approached are rendered unsuitable owing to the nature of the particular modification. Therefore, there are needs for improved anti-FVIII antibodies and immunoaffinity purification methods.

### BRIEF SUMMARY OF THE INVENTION

The present invention provides an anti-FVIII antibody or antigen-binding molecule thereof which specifically binds to a FVIII epitope, wherein the antibody or antigen-binding molecule thereof exhibits one or more of the following characteristics:

(a) the anti-FVIII antibody or antigen-binding molecule thereof specifically binds to the same FVIII epitope as an antibody selected from MBS11, MBS32, MBS22, MBS14, GMA8023, GMA8024, GMA8045, GMA8025, GMA5G8, GMA8026, or MBS17;
(b) the anti-FVIII antibody or antigen-binding molecule thereof competitively inhibits FVIII binding by an antibody selected from MBS11, MBS32, MBS22, MBS14, GMA8023, GMA8024, GMA8045, GMA8025, GMA5G8, GMA8026, or MBS17; or
(c) the anti-FVIII antibody or antigen-binding molecule thereof comprises at least one, at least two, at least three, at least four, or at least five complementarity determining regions (CDR) or variants thereof of an antibody selected from MBS11, MBS32, MBS22, MBS14, GMA8023, GMA8024, GMA8045, GMA8025, GMA5G8, GMA8026, or MBS17,
wherein the FVIII epitope is located in an A2 domain, an A3 domain, a C1 domain, a C2 domain, or any combinations thereof, and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In one embodiment, an anti-FVIII antibody or antigen-binding molecule thereof which specifically binds to a FVIII epitope comprises:

(i) a variable heavy chain CDR-1 (VH-CDR1) sequence at least about 60%, 70%, 80%, 90%, 95%, or 100% identical to a VH-CDR1 of an antibody selected from MBS11, MBS32, GMA8023, GMA8024, or MBS22;

(ii) a variable heavy chain CDR-2 (VH-CDR2) sequence at least about 60%, 70%, 80%, 90%, 95%, or 100% identical to a VH-CDR2 of an antibody selected from MBS11, MBS32, GMA8023, GMA8024, or MBS22;

(iii) a variable heavy chain CDR-3 (VH-CDR3) sequence at least about 60%, 70%, 80%, 90%, 95%, or 100% identical to a VH-CDR3 of an antibody selected from MBS11, MBS32, GMA8023, GMA8024, or MBS22;

(iv) a variable light chain CDR-1 (VL-CDR1) sequence at least about 60%, 70%, 80%, 90%, 95%, or 100% identical to a VL-CDR1 of an antibody selected from MBS11, MBS32, GMA8023, GMA8024, or MBS22;

(v) a variable light chain CDR-2 (VL-CDR2) sequence at least about 60%, 70%, 80%, 90%, 95%, or 100% identical to a VL-CDR2 of an antibody selected from MBS11, MBS32, GMA8023, GMA8024, or MBS22; and,

(vi) a variable light chain CDR-3 (VL-CDR3) sequence at least about 60%, 70%, 80%, 90%, 95%, or 100% identical to a VL-CDR3 of an antibody selected from MBS11, MBS32, GMA8023, GMA8024, or MBS22, wherein the FVIII epitope is located in an A2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In another embodiment, an anti-FVIII antibody or antigen-binding molecule thereof, which specifically binds to an FVIII epitope, comprises a VH region, which comprises an amino acid sequence at least about 80%/c, 85%, 90%, 95%, or 100% identical to a VH of an antibody selected from MBS11, MBS32, GMA8023, GMA8024, or MBS22 and a VL region, which comprises an amino acid sequence at least about 80%, 85%, 90%, 95%, or 100% identical to a VL of an antibody selected from MBS11, MBS32, GMA8023, GMA8024, or MBS22, wherein the FVIII epitope is located in an A2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof comprises the VH of MBS11 and the VL of MBS11 (MBS11 antibody). In still other embodiments, an anti-FVIII antibody or antigen-binding molecule thereof comprises the VH of MBS32 and the VL of MBS32 (MBS32 antibody). In yet other embodiments, an anti-FVIII antibody or antigen-binding molecule thereof comprises the VH of MBS22 and the VL of MBS22 (MBS22 antibody). In certain embodiments, an anti-FVIII antibody or antigen-binding molecule thereof comprises the VH of GMA8023 and the VL of GMA8023. In some embodiments, an anti-FVIII antibody or antigen-binding molecule thereof comprises the VH of GMA8024 and the VL of GMA8024.

In some embodiments, an anti-FVIII antibody or antigen-binding molecule thereof, which specifically binds to a FVIII epitope, comprises:

(i) a variable heavy chain CDR-1 (VH-CDR1) sequence at least about 60%, 70%, 80%, 90%, 95%, or 100% identical to a VH-CDR1 of MBS14;

(ii) a variable heavy chain CDR-2 (VH-CDR2) sequence at least about 60%, 70%, 80%, 90%, 95%, or 100% identical to a VH-CDR2 of MBS14;

(iii) a variable heavy chain CDR-3 (VH-CDR3) sequence at least about 60%, 70%, 80%, 90%, 95%, or 100% identical to a VH-CDR3 of MBS14;

(iv) a variable light chain CDR-1 (VL-CDR1) sequence at least about 60%, 70%, 80%, 90%, 95%, or 100% identical to a VL-CDR1 of MBS14;

(v) a variable light chain CDR-2 (VL-CDR2) sequence at least about 60%, 70%, 80%, 90%, 95%, or 100% identical to a VL-CDR2 of MBS14; and,

(vi) a variable light chain CDR-3 (VL-CDR3) sequence at least about 60%, 70%, 80%, 90%, 95%, or 100% identical to a VL-CDR3 of MBS14, wherein the FVIII epitope is located in a C1 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In certain embodiments, an anti-FVIII antibody or antigen-binding molecule thereof, which specifically binds to a FVIII epitope, comprises a VH region, which comprises an amino acid sequence at least about 80%, 85%, 90%, 95%, or 100% identical to the VH of MBS14 and a VL region, which comprises an amino acid sequence at least about 80%, 85%, 90%, 95%, or 100% identical to the VL of MBS14, wherein the FVIII epitope is a C1 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In some embodiments, an anti-FVIII antibody or antigen-binding molecule thereof, which specifically binds to a FVIII epitope, comprises:

(i) a variable heavy chain CDR-1 (VH-CDR1) sequence at least about 60%, 70%, 80%, 90%, 95%, or 100% identical to a VH-CDR1 of MBS17, GMA8045, GMA8025, GMA5G8, or GMA8026;

(ii) a variable heavy chain CDR-2 (VH-CDR2) sequence at least about 60%, 70%, 80%, 90%, 95%, or 100% identical to a VH-CDR2 of MBS17, GMA8045, GMA8025, GMA5G8, or GMA8026;

(iii) a variable heavy chain CDR-3 (VH-CDR3) sequence at least about 60%, 70%, 80%, 90%, 95%, or 100% identical to a VH-CDR3 of MBS17, GMA8045, GMA8025, GMA5G8, or GMA8026;

(iv) a variable light chain CDR-1 (VL-CDR1) sequence at least about 60%, 70%, 80%, 90%, 95%, or 100% identical to a VL-CDR1 of MBS17, GMA8045, GMA8025, GMA5G8, or GMA8026;

(v) a variable light chain CDR-2 (VL-CDR2) sequence at least about 60%, 70%, 80%, 90%, 95%, or 100% identical to a VL-CDR2 of MBS17, GMA8045, GMA8025, GMA5G8, or GMA8026; and,

(vi) a variable light chain CDR-3 (VL-CDR3) sequence at least about 60%, 70%, 80%, 90%, 95%, or 100% identical to a VL-CDR3 of MBS17, GMA8045, GMA8025, GMA5G8, or GMA8026,

wherein the FVIII epitope is located in a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In certain embodiments, an anti-FVIII antibody or antigen-binding molecule thereof, which specifically binds to a FVIII epitope, comprises a VH region, which comprises an amino acid sequence at least about 80%, 85%, 90%, 95%, or 100% identical to the VH of MBS17, GMA8045, GMA8025, GMA5G8, or GMA8026 and a VL region, which comprises an amino acid sequence at least about 80%, 85%, 90%, 95%, or 100% identical to the VL of MBS17, GMA8045, GMA8025, GMA5G8, or GMA8026, wherein the FVIII epitope is located in a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof is a monoclonal antibody, a chimeric antibody, or a humanized antibody. In still other embodiments, an anti-FVIII antibody or antigen-binding molecule thereof is: (a) a single chain Fv (“scFv”); (b) a diabody; (c) a minibody; (d) a polypeptide chain of an antibody; (e) F(ab′)₂; or (f) F(ab).

In yet other embodiments, the invention includes a nucleic acid molecule or a set of nucleic acid molecules encoding an anti-FVIII antibody or antigen-binding molecule or a complement thereof, a vector or a set of vectors comprising the nucleic acid molecule or the set of the nucleic acid molecules or a complement thereof, a host cell comprising the vector or the set of vectors, a composition comprising an anti-FVIII antibody or antigen-binding molecule thereof, the nucleic acid molecule or the set of nucleic acid molecules, the vector or the set of vectors and a carrier, or a kit comprising an anti-FVIII antibody or antigen-binding molecule thereof, the nucleic acid molecule or the set of nucleic acid molecules, or the vector or the set of vectors and a packaging material.

In some embodiments, the invention provides a method for producing an anti-FVIII antibody or antigen-binding molecule thereof comprising culturing the host cell and recovering the anti-FVIII antibody or antigen-binding molecule thereof from the culture medium.

Also provided is a method of purifying a FVIII protein, comprising contacting an anti-FVIII antibody or antigen-binding molecule thereof, which specifically binds to a FVIII epitope, with the FVIII protein and eluting the FVIII protein in a buffer, wherein the anti-FVIII antibody or antigen-binding molecule thereof comprises:

(i) a variable heavy chain CDR-1 (VH-CDR1) sequence at least about 60%, 70%, 80%, 90%, 95%, or 100% identical to a VH-CDR1 of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006;

(ii) a variable heavy chain CDR-2 (VH-CDR2) sequence at least about 60%, 70%, 80%, 90%, 95%, or 100% identical to a VH-CDR2 of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006;

(iii) a variable heavy chain CDR-3 (VH-CDR3) sequence at least about 60%, 70%, 80%, 90%, 95%, or 100% identical to a VH-CDR3 of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006;

(iv) a variable light chain CDR-1 (VL-CDR1) sequence at least about 60%, 70%, 80%, 90%, 95%, or 100% identical to a VL-CDR1 of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006;

(v) a variable light chain CDR-2 (VL-CDR2) sequence at least about 60%, 70%, 80%, 90%, 95%, or 100% identical to a VL-CDR2 of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006; and,

(vi) a variable light chain CDR-3 (VL-CDR3) sequence at least about 60%, 70%, 80%, 90%, 95%, or 100% identical to a VL-CDR3 of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006,

wherein the FVIII epitope is located in an A1 domain, an A2 domain, an A3 domain, a C1 domain, a C2 domain, a light chain, or any combinations thereof and wherein the buffer comprises propylene glycol and arginine.

In one aspect, an anti-FVIII antibody or antigen-binding molecule thereof useful for the method comprises a VH region and a VL region, wherein the VH region comprises an amino acid sequence at least 60%, at least 70%, at least 80%, at least 90%, at least 95%, or at least 100% identical to a VH region of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006 and wherein the VL region comprises an amino acid sequence at least 60%, at least 70%, at least 80%, at least 90%, at least 95%, or at least 100% identical to a VL region of an antibody selected from GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006.

In another aspect, the FVIII epitope to which the anti-FVIII antibody or antigen-binding molecule thereof binds is located in an A1 domain. In one example, the anti-FVIII antibody or antigen-binding molecule thereof comprises a VH region and a VL region, wherein the VH region comprises the VH of GMA8002 and the VL region comprises the VL of GMA8002.

In other aspects, the FVIII epitope to which the anti-FVIII antibody or antigen-binding molecule thereof binds is located in an A2 domain. For example, the anti-FVIII antibody or antigen-binding molecule thereof comprises a VH region and a VL region, wherein the VH region comprises the VH of an antibody selected from GMA8021, GMA8016, MBS32, N772-10M, GMA012, GMA8023, GMA8024, or OBT-0037A and the VL region comprises the VL of an antibody selected from GMA8021, GMA8016, MBS32, N772-10M, GMA012, GMA8023, GMA8024, or OBT-0037A.

In some aspects, the FVIII epitope to which the anti-FVIII antibody or antigen-binding molecule thereof binds is located in an A3 domain. Non-limiting examples of the anti-FVIII antibody or antigen-binding molecule thereof include an antibody or antigen-binding molecule thereof comprising a VH region and a VL region, wherein the VH region comprises the VH of GMA8001 or MBS14 and the VL region comprises the VL of GMA8001 or MBS14.

In other aspects, the FVIII epitope to which the anti-FVIII antibody or antigen-binding molecule thereof binds is located in a C1 domain. For example, the anti-FVIII antibody or antigen-binding molecule thereof comprises a VH region and a VL region, wherein the VH region comprises the VH of GMA8011 and the VL region comprises the VL of GMA8011.

In certain aspects, the FVIII epitope to which the anti-FVIII antibody or antigen-binding molecule thereof binds is located in a C2 domain. For example, the anti-FVIII antibody or antigen-binding molecule thereof comprises a VH region and a VL region, wherein the VH region comprises the VH of GMA8006, GMA5G8, or GMA8026 and the VL region comprises the VL of GMA 8006, GMA5G8, or GMA8026.

In some aspects, the FVIII epitope to which the anti-FVIII antibody or antigen-binding molecule thereof binds is located in a light chain of the FVIII protein. For example, the anti-FVIII antibody or antigen-binding molecule thereof comprises a VH region and a VL region, wherein the VH region comprises the VH of GMA8020, GMA5E8, or GMA8013 and the VL region comprises the VL of GMA8020, GMA5E8, or GMA8013.

The instant disclosure provides that the anti-FVIII antibody or antigen-binding molecule thereof can bind to the FVIII protein at a dissociation constant (K_{D}) lower than about 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, 0.005 nM, or 0.001 nM.

In one embodiment of the invention, the buffer that the anti-FVIII antibody or antigen-binding molecule thereof is eluted comprises at least about 30% (v/v), at least about 40% (v/v), at least about 45% (v/v), at least about 50% (v/v), at least about 55% (v/v), at least about 60% (v/v), at least about 65% (v/v), at least about 70% (v/v), or at least about 75% (v/v) propylene glycol. In another embodiment, the buffer is an elution buffer comprising at least about 0.5M, at least about 0.6M, at least about 0.7M, at least about 0.8M, at least about 0.9M, at least about 1.0M, at least about 1.1M, at least about 1.2M, or at least about 1.3M arginine. In other embodiments, the buffer further comprises histidine, CaCl₂, Tween-20, or any combinations thereof. In a particular embodiment, the buffer comprises about 50 mM histidine, about 0.9M arginine, about 50 mM CaCl₂, about 45% (v/v) propylene glycol, and about 0.05% Tween-20 at pH 7.2.

The invention also includes a method of purifying a FVIII protein, comprising contacting an anti-FVIII antibody or antigen-binding molecule thereof, which specifically binds to a FVIII epitope, with the FVIII protein and eluting the FVIII protein in a high ionic buffer, wherein the anti-FVIII antibody or antigen-binding molecule thereof comprises:

(i) a variable heavy chain CDR-1 (VH-CDR1) sequence at least about 60%, 70%, 80%, 90%, 95%, or 100% identical to a VH-CDR1 of an antibody selected from ESH4, GMA8013, MBS17, GMA5G8, GMA5E8, GMA8025, or OBT0037A;

(ii) a variable heavy chain CDR-2 (VH-CDR2) sequence at least about 60%, 70%, 80%, 90%, 95%, or 100% identical to a VH-CDR2 of an antibody selected from ESH4, GMA8013, MBS17, GMA5G8, GMA5E8, GMA8025, or OBT0037A;

(iii) a variable heavy chain CDR-3 (VH-CDR3) sequence at least about 60%, 70%, 80%, 90%, 95%, or 100% identical to a VH-CDR3 of an antibody selected from ESH4, GMA8013, MBS17, GMA5G8, GMA5E8, GMA8025, or OBT0037A;

(iv) a variable light chain CDR-1 (VL-CDR1) sequence at least about 60%, 70%, 80%, 90%, 95%, or 100% identical to a VL-CDR1 of an antibody selected from ESH4, GMA8013, MBS17, GMA5G8, GMA5E8, GMA8025, or OBT0037A;

(v) a variable light chain CDR-2 (VL-CDR2) sequence at least about 60%, 70%, 80%, 90%, 95%, or 100% identical to a VL-CDR2 of an antibody selected from ESH4, GMA8013, MBS17, GMA5G8, GMA5E8, GMA8025, or OBT0037A; and,

(vi) a variable light chain CDR-3 (VL-CDR3) sequence at least about 60%, 70%, 80%, 90%, 95%, or 100% identical to a VL-CDR3 of an antibody selected from ESH4, GMA8013, MBS17, GMA5G8, GMA5E8, GMA8025, or OBT0037A, wherein the FVIII epitope is located in a light chain, an A2 region, a C2 domain, or any combinations thereof

In one embodiment, the anti-FVIII antibody or antigen-binding molecule thereof comprises a VH region and a VL region, wherein the VH region comprises an amino acid sequence at least 60%, at least 70%, at least 80%, at least 90%, at least 95%, or at least 100% identical to a VH region of an antibody selected from ESH4, GMA8013, MBS17, GMA5G8, GMA5E8, GMA8025, or OBT0037A and wherein the VL region comprises an amino acid sequence at least 60%, at least 70%, at least 80%, at least 90%, at least 95%, or at least 100% identical to a VL region of an antibody selected from ESH4, GMA8013, MBS17, GMA5G8, GMA5E8, GMA8025, or OBT0037A. In another embodiment, the high ionic buffer comprises NaCl, CaCl2, Tris-HCl, or any combinations thereof. In other embodiments, the high ionic buffer comprises at least about 5 nM, at least about 10 nM, at least about 15 nM, at least about 20 nM, at least about 25 nM, at least about 30 nM, at least about 40 nM, at least about 50 nM, at least about 60 nM, at least about 70 nM, at least about 80 nM Tris-HCl. In still other embodiments, the high ionic buffer comprises at least about 0.1M, at least about 0.2M, at least about 0.3M, at least about 0.4M, at least about 0.5M, at least about 0.6M, at least about 0.7M, at least about 0.8M, at least about 0.9M, at least about 1.0M, at least about 1.1M, at least about 1.2M, at least about 1.3M, at least about 1.4M, or at least about 1.5M NaCl. In yet other embodiments, the high ionic buffer comprises at least about 0.1M CaCl2, at least about 0.15M, at least about 0.2M, at least about 0.25M, at least about 0.3M, at least about 0.35M, at least about 0.4M, at least about 0.45M, at least about 0.5M, at least about 0.6M, at least about 0.65M, at least about 0.7M, at least about 0.75M, at least about 0.8M, at least about 0.85M, at least about 0.9M, at least about 0.95M, or at least about 1.0M CaCl₂. In a specific embodiment, wherein the high ionic buffer comprises about 20 nM Tris-HCl, about 0.6M NaCl, about 0.35M CaCl₂ at pH 7.2.

The invention also provides a method of reducing or preventing a FVIII protein from binding to von Willebrand Factor (“VWF”) comprising contacting an anti-FVIII antibody or antigen-binding molecule thereof with the FVIII protein, wherein the anti-FVIII antibody or antigen-binding molecule thereof binds to the FVII protein at a VWF binding site. In one embodiment, the invention includes a method of identifying a FVIII protein that does not bind to VWF comprising contacting an anti-FVIII antibody or antigen-binding molecule thereof with the FVIII protein, measuring the binding of the anti-FVIII antibody or antigen-binding molecule thereof to the FVIII protein, and isolating the FVIII protein that does not bind to the anti-FVIII antibody or antigen-binding molecule thereof.

In one embodiment, the anti-FVIII antibody or antigen-binding molecule thereof comprises:

(i) a variable heavy chain CDR-1 (VH-CDR1) sequence at least about 60%, 70%, 80%, 90%, 95%, or 100% identical to VH-CDR1 of an antibody selected from GMA8018, MBS17, ESH4, GMA8013, GMA8008, GMA8011, GMA8045, or GMA8020;

(ii) a variable heavy chain CDR-2 (VH-CDR2) sequence at least about 60%, 70%, 80%, 90%, 95%, or 100% identical to VH-CDR2 of an antibody selected from GMA8018, MBS17, ESH4, GMA8013, GMA8008, GMA8011, GMA8045, or GMA8020;

(iii) a variable heavy chain CDR-3 (VH-CDR3) sequence at least about 60%, 70%, 80%, 90%, 95%, or 100% identical to VH-CDR3 of an antibody selected from GMA8018, MBS17, ESH4, GMA8013, GMA8008, GMA8011, GMA8045, or GMA8020;

(iv) a variable light chain CDR-1 (VL-CDR1) sequence at least about 60%, 70%, 80%, 90%, 95%, or 100% identical to VL-CDR1 of an antibody selected from GMA8018, MBS17, ESH4, GMA8013, GMA8008, GMA8011, GMA8045, or GMA8020;

(v) a variable light chain CDR-2 (VL-CDR2) sequence at least about 60%, 70%, 80%, 90%, 95%, or 100% identical to VL-CDR2 of an antibody selected from GMA8018, MBS17, ESH4, GMA8013, GMA8008, GMA8011, GMA8045, or GMA8020; and,

(vi) a variable light chain CDR-3 (VL-CDR3) sequence at least about 60%, 70%, 80%, 90%, 95%, or 100% identical to VL-CDR3 of an antibody selected from GMA8018, MBS17, ESH4, GMA8013, GMA8008, GMA8011, GMA8045, or GMA8020, and

wherein the anti-FVIII antibody or antigen-binding molecule thereof specifically binds to a VWF binding site on the FVIII protein.

In another embodiment, the anti-FVIII antibody or antigen-binding molecule thereof comprises a VH region and a VL region,

wherein the VH region comprises an amino acid sequence at least 60%, at least 70%, at least 80%, at least 90%, at least 95%, or at least 100% identical to a VH region of an antibody selected from GMA8018, MBS17, ESH4, GMA8013, GMA8008, GMA8011, GMA8045, or GMA8020;

wherein the VL region comprises an amino acid sequence at least 60%, at least 70%, at least 80%, at least 90%, at least 95%, or at least 100% identical to a VL region of an antibody selected from GMA8018, MBS17, ESH4, GMA8013, GMA8008, GMA8011, GMA8045, or GMA8020; and

wherein the anti-FVIII antibody or antigen-binding molecule thereof specifically binds to a VWF binding site on the FVIII protein.

In one aspect, the invention includes a method of reducing or preventing a FVIII-binding molecule from binding to a FVIII protein comprising contacting an anti-FVIII antibody or antigen-binding molecule thereof with the FVIII protein, wherein the anti-FVIII antibody or antigen-binding molecule thereof binds to a FVIII-binding site to which the FVIII-binding molecules binds. In another aspect, the invention includes a method of identifying a subject who has developed a FVIII inhibitor which binds to a FVIII protein in plasma comprising contacting an anti-FVIII antibody or antigen-binding molecule thereof with the plasma of the subject, wherein the anti-FVIII antibody or antigen-binding molecule thereof binds to a FVIII-binding site to which the FVIII inhibitor binds. In other aspects, the invention includes a method of identifying a FVIII binding site of a FVIII inhibitor comprising contacting an anti-FVIII antibody or antigen-binding molecule thereof with a FVII protein in the presence of the FVIII inhibitor. Also provided is a method of preventing or inhibiting a cellular uptake of a FVIII protein comprising contacting an anti-FVIII antibody or antigen-binding molecule thereof with the FVIII protein which specifically binds to a FVIII epitope.

In one embodiment, wherein the anti-FVIII antibody or antigen-binding molecule thereof comprises:

(i) a variable heavy chain CDR-1 (VH-CDR1) sequence at least about 60%, 70%, 80%, 90%, 95%, or 100% identical to VH-CDR1 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;

(ii) a variable heavy chain CDR-2 (VH-CDR2) sequence at least about 60%, 70%, 80%, 90%, 95%, or 100% identical to VH-CDR2 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;

(iii) a variable heavy chain CDR-3 (VH-CDR3) sequence at least about 60%, 70%, 80%, 90%, 95%, or 100% identical to VH-CDR3 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;

(iv) a variable light chain CDR-1 (VL-CDR1) sequence at least about 60%, 70%, 80%, 90%, 95%, or 100% identical to VL-CDR1 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;

(v) a variable light chain CDR-2 (VL-CDR2) sequence at least about 60%, 70%, 80%, 90%, 95%, or 100% identical to VL-CDR2 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17; and,

(vi) a variable light chain CDR-3 (VL-CDR3) sequence at least about 60%, 70%, 80%, 90%, 95%, or 100% identical to VL-CDR3 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17, and

wherein the anti-FVIII antibody or antigen-binding molecule thereof specifically binds to a FVIII epitope, which is located in an A1 region, an A2 region, an A3 region, a C1 region, a C2 region, or any combinations thereof.

In another embodiment, the anti-FVIII antibody or antigen-binding molecule thereof comprises a VH region and a VL region,

wherein the VH region comprises an amino acid sequence at least 60%, at least 70%, at least 80%, at least 90%, at least 95%, or at least 100% identical to the VH region of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17; and

wherein the VL region comprises an amino acid sequence at least 60%, at least 70%, at least 80%, at least 90%, at least 95%, or at least 100% identical to the VL region of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17.

### BRIEF DESCRIPTION OF THE DRAWINGS/FIGURES

FIG. 1 shows pairwise epitope overlap analysis using Octet QK384. Anti-mouse Fv biosensor probe is used to bind the first antibody, which captures FVIII. The FVIII bound probe is then exposed to a second antibody.

FIG. 2A shows a schematic diagram of BDD-rFVIII dissociation by EDTA into the heavy chain (HC) and the light chain (LC). FIG. 2B shows the spectral interference signal (nm).

FIG. 3A shows a schematic diagram of BDD-rFVIII treatment with EDTA and then α-thrombin. FIG. 3B shows the spectral interference signal (nm). (*) next to the numbers indicates that there are no additional binding of the antibodies to different chains or domains. Others indicate that there are additional binding, e.g., to the same chain or domain.

FIGS. 4A and 4B show the spectral interference signal to test application of the FVIII antibodies for immunoaffinity purification. Test antibodies [GMA8011 in FIG. 4A and GMA8015 in FIG. 4B] were bound to anti-mouse Fv biosensor probes. The probes were then exposed to an elution buffer for preconditioning, and FVIII was then captured by the antibodies bound to the probes. Each probe was then exposed to elution buffer. FIG. 4A shows complete elution, and FIG. 4B shows no elution.

FIG. 5 shows epitope overlap analysis of the anti-FVIII antibodies. The solid lines between MBS22 and 8016, between MBS14 and VIIISELECT®, between VIIISELECT® and 8011, between ESH4 and MBS17, and between VIIISELECT® and 8020 indicate partially overlapping epitopes. The other solid lines indicate overlapping epitopes. The dotted lines indicate non-overlapping epitopes. The halo rings indicate the antibodies that release FVIII in buffers while maintaining FVIII activity.

FIG. 6A shows the affinity of anti-FVIII antibodies for rFVIIIFc and BDD rFVIII as determined by surface plasmon resonance (SPR). The affinity data for 8014, 8020, 8011, and MBS22 are shown. The affinity was evaluated by using a ProteOn XPR36 SPR array instrument. Each antibody was tested three times for binding to BDD rFVIII and rFVIIIFc. FIG. 6B shows the affinity of MBS22 for rFVIIIFc and BDD rFVIII as determined with Biacore T100 SPR instrument with direct conjugation of the antibody to the chip and by using long dissociation step (35 minutes).

FIG. 7 shows 2-D plot of the affinities of the antibodies for rFVIIIFc (y-axis) and BDD rFVIII (x-axis). Affinities are expressed in terms of K_{D} (M). Values represent mean±SD for triplicate experiments.

FIG. 8A (top panel) shows negative stain electron microscopy (EM) image of GMA-8015 Fab-bound rFVIII grouped into 25 different classes. FIG. 8A (lower panel) shows a representative image of a single class GMA-8015 Fab bound rFVIII. FIG. 8B (top panel) shows negative strain EM image of ESH8 Fab-bound rFVIII grouped into 25 different classes. FIG. 8B (lower panel) shows a representative image of a single class ESH8 Fab-bound rFVIII.

FIG. 9A (top panel) shows negative stain EM images of rFVIIIFc grouped into 100 different classes. FIG. 9A (lower panel) shows a representative image of a single class of rFVIIIFc. FIG. 9B (top panel) shows 6 groups representing different conformations of rFVIIIFc, including 3D maps calculated from these groups. FIG. 9B (lower panel) shows one of the possible orientations of Fc relative to the FVIII component of rFVIIIFc from different views.

FIG. 10A shows representative negative stain EM image of ESH8 Fab-bound rFVIII. FIG. 10B shows representative negative stain EM image of ESH8 Fab-bound rFVIIIFc. FIG. 10C shows one of the 3D reconstructions of ESH8 Fab-bound rFVIIIFc.

FIG. 10D shows the affinity of ESH8 Fab for rFVIII and rFVIIIFc. Affinities are expressed in terms of K_{D} (nM).

FIG. 11 shows pairwise epitope overlap analysis using Octet QK384. Anti-mouse Fv biosensor probe is used to bind the first antibody, which captures FVIII. The FVIII bound probe is then exposed to a second antibody.

FIG. 12 shows epitope overlap analysis of the anti-FVIII antibodies using the SPR method.

### DETAILED DESCRIPTION

In order to provide a clear understanding of the specification and claims, the following definitions are provided below.

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising,” otherwise analogous embodiments described in terms of “consisting of” and/or “consisting essentially of” are also provided.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art relevant to the range or element at issue.

The amount of broadening from the strict numerical boundary depends upon many factors. For example, some of the factors to be considered can include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” can mean±5%, or +10%, depending on the relevant technology. Also, the disclosure of ranges is intended as a continuous range including every value between the minimum and maximum values recited.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxyl orientation. The headings provided herein are not limitations of the various embodiments of the disclosure, which can be by reference to the specification as a whole. Accordingly, the terms defined immediately below are more fully defined by reference to the specification in its entirety. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “amino acid chain,” or any other term used to refer to a chain or chains of two or more amino acids, are included within the definition of “polypeptide,” and the term “polypeptide” can be used instead of, or interchangeably with any of these terms.

As used herein the term “protein” is intended to encompass a molecule comprised of one or more polypeptides, which can in some instances be associated by bonds other than amide bonds.

Polypeptides can be either monomers or multimers. For example, in one embodiment, an antibody, an antigen-binding molecule thereof, or a chimeric molecule of the invention can be a dimeric polypeptide. A dimeric antibody, an antigen-binding molecule thereof can comprise two polypeptide chains or can consist of one polypeptide chain (e.g., in the case of a sckc molecule). In one embodiment, the dimers can be a homodimer, comprising two identical monomeric subunits or polypeptides (e.g., two identical Fc moieties or two identical biologically active moieties). In another embodiment, the dimers are heterodimers, comprising two non-identical monomeric subunits or polypeptides (e.g., comprising two different clotting factors or portions thereof or one clotting factor only). See, e.g., U.S. Pat. No. 7,404,956, incorporated herein by reference.

The terms “polypeptide” and “protein” are also intended to refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

A polypeptide which is “isolated” is a polypeptide which is in a form not found in nature. Isolated polypeptides include those which have been purified to a degree that they are no longer in a form in which they are found in nature. In some embodiments, a polypeptide which is isolated is substantially pure.

“Derivatives” of anti-FVIII antibodies or antigen-binding molecules thereof of the invention are polypeptides or proteins which have been altered so as to exhibit additional features not found on the native polypeptide or protein. Also included as “derivatives” are those peptides that contain one or more naturally occurring amino acid derivatives of the twenty standard amino acids. A polypeptide or amino acid sequence “derived from” a designated polypeptide or protein refers to the origin of the polypeptide. In one embodiment, the polypeptide or amino acid sequence which is derived from a particular sequence has an amino acid sequence that is essentially identical to that sequence or a portion thereof, wherein the portion consists of at least about 10 to about 20 amino acids, at least about 20 to about 30 amino acids, or at least about 30 to about 50 amino acids, or which is otherwise identifiable to one of ordinary skill in the art as having its origin in the sequence.

Polypeptides that are “variants” of another polypeptide can have one or more mutations relative to the starting polypeptide, e.g., one or more amino acid residues which have been substituted with another amino acid residue or which has one or more amino acid residue insertions or deletions. In one embodiment, the polypeptide comprises an amino acid sequence which is not naturally occurring. Such variants necessarily have less than 100% sequence identity or similarity with the starting polypeptide. In another embodiment, the variant will have an amino acid sequence from about 75% to less than 100% amino acid sequence identity or similarity with the amino acid sequence of the starting polypeptide, for example, from about 80% to less than 100%, from about 85% to less than 100%, from about 90% to less than 100% (e.g., 91%, 92%, 93%, 94%, 95%, 96%, 97%, 98%, 99%) and from about 95% to less than 100%, e.g., over the length of the variant molecule. In one embodiment, there is one amino acid difference between a starting polypeptide sequence and the sequence derived therefrom.

The term “sequence” as used to refer to a protein sequence, a peptide sequence, a polypeptide sequence, or an amino acid sequence means a linear representation of the amino acid constituents in the polypeptide in an amino-terminal to carboxyl-terminal direction in which residues that neighbor each other in the representation are contiguous in the primary structure of the polypeptide.

The term “amino acid” includes alanine (Ala or A); arginine (Arg or R); aspar-agine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V).

Non-traditional amino acids are also within the scope of the invention and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, and C). These amino acids can participate in hydrophobic interactions and electrostatic interactions. As used herein, the term “charged amino acid” includes amino acids that can have non-zero net charge on their side chains (e.g. R, K, H, E, and D). These amino acids can participate in hydrophobic interactions and electrostatic interactions.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions”, can be made, e.g. insertion of about three to about five or even up to about ten, fifteen, or twenty amino acid residues. The inserted residue(s) can be naturally occurring or non-naturally occurring as disclosed above. An “amino acid deletion” refers to the removal of at least one amino acid residue from a predetermined amino acid sequence.

A “conservative amino acid substitution” is one in which the amino acid residue is replaced with an amino acid residue having a similar side chain. Families of amino acid residues having similar side chains have been defined in the art, including basic side chains (e.g., Lys, Arg, and His), acidic side chains (e.g., Asp and Glu), uncharged polar side chains (e.g., Gly, Asn, Gnl, Ser, Thr, Tyr, and Cys), nonpolar side chains (e.g., Ala, Val, Leu, Ile, Pro, Phe, Met, and Trp), beta-branched side chains (e.g., Thr, Val, and Ile) and aromatic side chains (e.g., Tyr, Phe, Trp, and His). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

Non-conservative substitutions include those in which (i) a residue having an electropositive side chain (e.g., Arg, His, or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, He, Phe, or Val), (iii) a cysteine or proline is substituted for, or by, any other residue, or (iv) a residue having a bulky hydrophobic or aromatic side chain (e.g., Val, He, Phe, or Trp) is substituted for, or by, one having a smaller side chain (e.g., Ala or Ser) or no side chain (e.g., Gly).

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences.

One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org (ClustalX is a version of the ClustalW2 program ported to the Windows environment). Another suitable program is MUSCLE, available from www.drive5.com/muscle. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

In one embodiment, the antibodies and antigen-binding molecules thereof, as well as the chimeric molecules of the invention can comprise an amino acid sequence derived from a human protein sequence. However, the antibodies and antigen-binding molecules thereof, as well as the chimeric molecules of the invention can comprise one or more amino acids from another mammalian species. In a particular embodiment, the antibodies and antigen-binding molecules thereof, as well as the chimeric molecules of the invention are not immunogenic.

As used herein, the terms “linked,” “fused”, or “fusion” refer to linkage via a peptide bonds (e.g., genetic fusion), chemical conjugation, or other means known in the art. For example, one way in which molecules or moieties can be linked employs peptide linkers, which link the molecules or moieties via peptide bonds. The terms “genetically fused,” “genetically linked,” or “genetic fusion” are used interchangeably and refer to the co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence.

Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs (which segments are not normally so joined in nature). In this case, the single polypeptide is cleaved during processing to yield dimeric molecules comprising two polypeptide chains.

The term “antibody” means an immunoglobulin molecule that recognizes and specifically binds to a target, such as a protein (e.g., FVIII or a domain thereof,), polypeptide, peptide, carbohydrate, polynucleotide, lipid, or combinations of the foregoing through at least one antigen recognition site within the variable region of the immunoglobulin molecule.

A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CH1, CH2, and CH3. Each light chain is comprised of a “light chain variable region” or “light chain variable domain” (abbreviated herein as VL) and a light chain constant region. The light chain constant region is comprised of one domain, C1. The VH and VL regions can be further subdivided into regions of hypervariablity, termed Complementarity Determining Regions (CDR), interspersed with regions that are more conserved, termed framework regions (FW).

Each VH and VL region is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. As used herein, the term “antibody” encompasses intact polyclonal antibodies, intact monoclonal antibodies, antibody fragments (such as Fab, Fab′, F(ab′)2, and Fv fragments), single chain Fv (scFv), minibodies, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, radioisotopes, etc.

There are at least two techniques for determining the location of CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See e.g., Lefranc, M. P. et al., Dev. Comp. Immunol. 27: 55-77(2003). The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDR sequences described herein correspond to the classical Kabat numbering locations, namely Kabat VH-CDR1 is at positions 31-35, VH-CDR2 is a positions 50-65, and VH-CDR3 is at positions 95-102. VL-CDR1, VL-CDR2, and VL-CDR3 also correspond to classical Kabat numbering locations, namely positions 14-24, 50-56 and 89-97, respectively.

The term “consensus sequence,” as used herein with respect to a CDR in the light chain (VL) or heavy chain (VH) variable regions, refers to a composite or genericized amino acid sequence defined based on information as to which amino acid residues are present at a given position based in multiple sequence alignments. Thus, in a “consensus sequence” for a VL or VH chain CDR1, CDR2, or CDR3, certain amino acid positions are occupied by one of multiple possible amino acid residues at that position. For example, if an arginine (R) or a serine (S) occur at a particular position X, then that particular position within the consensus sequence can be either arginine or serine (R or S). Such occurrence would be represented, for example, as _{N}-Z1Z₂X_{Z}Zₜ₋₁Z_{t-C}, where Zₜ₋₁ are invariant amino acids in the multiple sequence aligment, X represent a position occupied by variant amino acids (e.g., R or S), and the subindex n is an ordinal. As used herein, referring to a polypeptide sequence as consisting of or comprising a consensus sequence means that the polypeptide sequence consists of or comprises one of the of multiple possible amino acid sequences represented by the consensus sequence.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced.

The term “Fab′” refers to an antibody fragment that is essentially equivalent to that obtained by reduction of the disulfide bridge or bridges joining the two heavy chain pieces in the F(ab′)2 fragment. Such fragments can be enzymatically or chemically produced by fragmentation of an intact antibody, recombinantly produced from a gene encoding the partial antibody sequence, or it can be wholly or partially synthetically produced.

The term “F(ab′)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced.

The term “Fv” refers to an antibody fragment that consists of one NH and one N domain held together by noncovalent interactions.

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, or Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides. The term “humanized antibody” refers to an antibody derived from a non-human (e.g., murine) immunoglobulin, which has been engineered to contain minimal non-human (e.g., murine) sequences. The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

In one embodiment, an anti-FVIII antibody of the invention comprises an antibody variant. The term “antibody variant” or “modified antibody” includes an antibody which does not occur in nature and which has an amino acid sequence or amino acid side chain chemistry which differs from that of a naturally-derived antibody by at least one amino acid or amino acid modification as described herein. As used herein, the term “antibody variant” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen; heavy chain molecules joined to scFv molecules; single-chain antibodies; diabodies; triabodies; and antibodies with altered effector function and the like.

As used herein the term “scFv” or “scFv molecule” includes binding molecules which consist of one light chain variable domain (VL) or a portion thereof, and one heavy chain variable domain (VH) or a portion thereof, wherein each variable domain (or a portion thereof) is derived from the same or different antibodies. Single chain Fv molecules preferably comprise an scFv linker interposed between the VH domain and the VL domain. Exemplary scFv molecules are known in the art and are described, for example, in U.S. Pat. No. 5,892,019; Ho et al., Gene 77:51 (1989); Bird et al., Science 242:423 (1988); Pantoliano et al., Biochemistry 30:10117 (1991); Milenic et al., Cancer Research 51:6363 (1991); Takkinen et al., Protein Engineering 4:837 (1991).

The term “scFv linker” as used herein refers to a moiety interposed between the VL and VH domains of the scFv. The scFv linkers preferably maintain the scFv molecule in an antigen-binding conformation. In one embodiment, a scFv linker comprises or consists of an scFv linker peptide. In certain embodiments, an scFv linker peptide comprises or consists of a gly-ser peptide linker. In other embodiments, an scFv linker comprises a disulfide bond.

As used herein, the term “antigen-binding molecule” refers to a molecule comprising an anti-FVIII antibody fragment, variant, or derivative thereof, comprising at least one CDR from one or more of the anti-FVIII antibodies disclosed herein. In some embodiments, the antigen-binding molecule is a protein. In other embodiments, the antigen-binding molecule is a protein scaffold (e.g., a fibronectin type III domain) or non-protein scaffold comprising at least one CDR from one of the anti-FVIII antibodies disclosed herein. In some embodiments, the antigen-binding molecule is an anti-FVIII antibody identified according to the methods disclosed herein, comprising at least one CDR identical to one of the CDR sequences disclosed herein. The term “antigen-binding molecule” also encompasses any molecule comprising a VH and/or VL region from one or more of the anti-FVIII antibodies disclosed herein. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

The term “polynucleotide” or “nucleotide” is intended to encompass a singular nucleic acid as well as plural nucleic acids and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. Examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid can include regulatory elements such as promoters, enhancers, ribosome binding sites, or transcription termination signals.

As used herein, a “coding region” or “coding sequence” is a portion of polynucleotide which consists of codons translatable into amino acids. Although a “stop codon” (tag, tga, or taa) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′terminus, encoding the carboxyl terminus of the resulting polypeptide.

Two or more coding regions of the present invention can be present in a single polynucleotide construct, e.g., on a single vector, or in separate polynucleotide constructs, e.g., on separate (different) vectors. It follows, then, that a single vector can contain just a single coding region, or comprise two or more coding regions, e.g., a single vector can separately encode a binding domain-A and a binding domain-B as described below. In addition, a vector, polynucleotide, or nucleic acid of the invention can encode heterologous coding regions, either fused or unfused to a nucleic acid encoding a binding domain of the invention. Heterologous coding regions include without limitation specialized elements or motifs, such as a secretory signal peptide or a heterologous functional domain.

The term “vector” or “expression vector” is used herein to mean vectors used in accordance with the present invention as a vehicle for introducing into and expressing a desired polynucleotide in a cell. As known to those skilled in the art, such vectors can easily be selected from the group consisting of plasmids, phages, viruses, and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems can be employed to produce the antibody, antigen-binding molecule thereof, or a chimeric molecule of the invention. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Additionally, cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. In one embodiment, an inducible expression system can be employed. Additional elements can also be needed for optimal synthesis of mRNA. These elements can include signal sequences, splice signals, as well as transcriptional promoters, enhancers, and termination signals. In one embodiment, a secretion signal, e.g., any one of several well characterized bacterial leader peptides (e.g., pelB, phoA, or ompA), can be fused in-frame to the N terminus of a polypeptide of the invention to obtain optimal secretion of the polypeptide. (Lei et al. (1988), Nature, 331:543; Better et al. (1988) Science, 240:1041; Mullinax et al., (1990). PNAS, 87:8095).

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

A “recombinant” polypeptide or protein refers to a polypeptide or protein produced via recombinant DNA technology. Recombinantly produced polypeptides and proteins expressed in host cells are considered isolated for the purpose of the invention, as are native or recombinant polypeptides which have been separated, fractionated, or partially or substantially purified by any suitable technique.

The term “host cell” refers to a cell that has been transformed with a vector constructed using recombinant DNA techniques and encoding at least one heterologous gene. In descriptions of processes for isolation of proteins from recombinant hosts, the terms “cell” and “cell culture” are used interchangeably to denote the source of protein unless it is clearly specified otherwise. In other words, recovery of protein from the “cells” can mean either from spun down whole cells, or from the cell culture containing both the medium and the suspended cells. The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, CHO cell line, BHK cell line, HEK cell line, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), PerC6 cells), HAK (hamster kidney line), SP2/O (mouse myeloma), P3x63-Ag3.653 (mouse myeloma), BFA-1c1BPT (bovine endothelial cells), and RAJI (human lymphocyte). Host cell lines are typically available from commercial services, the American Tissue Culture Collection or from published literature.

### II. Anti-FVIII Antibodies

The present invention provides antibodies and antigen-binding molecules thereof that specifically bind to a FVIII epitope. The FVIII antibodies or antigen-binding molecule thereof can bind to any one or more domains of full-length FVIII or BDD-rFVIII.

FVIII is known to contain an A1 domain, an a1 spacer region, an A2 domain, an a2 spacer region, an A3 domain, an a3 spacer region, a B domain, a C1 domain, and a C2 domain. Referring to the primary amino acid sequence position in mature full-length FVIII (SEQ ID NO: 1), the A1 domain of human FVIII extends from Ala1 to about Arg336, the a1 spacer region extends from about Met337 to about Arg372, the A2 domain extends from about Ser373 to about Tyr719, the a2 spacer region extends from about Glu720 to about Arg740, the B domain extends from about Ser741 to about Arg 1648, the a3 spacer region extends from about Glu1649 to about Arg1689, the A3 domain extends from about Ser1690 to about Asn2019, the C1 domain extends from about Lys2020 to about Asn2172, and the C2 domain extends from about Ser2173 to Tyr2332 (Saenko et al., J. Thromb. Hemostasis 1:922-930 (2005)). Other than specific proteolytic cleavage sites, designation of the locations of the boundaries between the domains and regions of FVIII can vary in different literature references. The boundaries noted herein are therefore designated as approximate by use of the term “about.” A polypeptide comprising the a3, A3, C1, and C2 domains, i.e., from about Ser1649 to Tyr2332, is cleaved from the polypeptide comprising the A1, a1, A2, a2, and B domains during normal FVIII processing resulting in a heavy chain and a light chain.

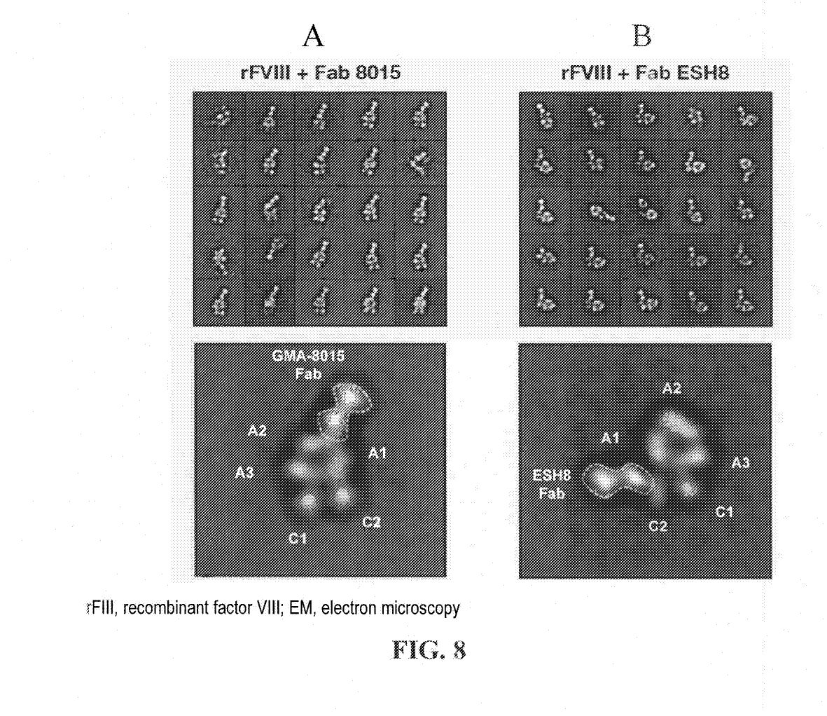

The B domain is not required for procoagulant activity, and in certain aspects, including commercially available therapeutic compositions, some or all of the B domain of FVIII is deleted (“B domain deleted factor VIII” or “BDD FVIII”). See, e.g., FIG. 8. An example of a BDD FVIII is REFACTO® or XYNTHA® (recombinant BDD FVIII), which comprises a first polypeptide corresponding to amino acids 1 to 743 of SEQ ID NO: 1, fused to a second polypeptide corresponding to amino acids 1638 to 2332 of SEQ ID NO: 1.

### II.A. Anti-FVIII Antibodies

The present invention provides an anti-FVIII antibody or antigen-binding molecule thereof which specifically binds to a Factor VIII (“FVIII”) epitope, wherein the antibody or antigen-binding molecule thereof specifically binds to the same FVIII epitope as an antibody selected from MBS11, MBS32, MBS22, MBS14, or MBS17 and wherein the FVIII epitope is located in an A2 domain, an A3 domain, a C1 domain, a C2 domain, or any combinations thereof and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. The invention also provides an anti-FVIII antibody or antigen-binding molecule thereof which specifically binds to a Factor VIII (“FVIII”) epitope, wherein the anti-FVIII antibody or antigen-binding molecule thereof competitively inhibits FVIII binding by an antibody selected from MBS11, MBS32, MBS22, MBS14, or MBS17, wherein the FVIII epitope is located in an A2 domain, an A3 domain, a C1 domain, a C2 domain, or any combinations thereof and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. The invention also includes an anti-FVIII antibody or antigen-binding molecule thereof which specifically binds to a Factor VIII (“FVIII”) epitope, wherein the anti-FVIII antibody or antigen-binding molecule thereof comprises at least one, at least two, at least three, at least four, or at least five complementarity determining regions (CDR) or variants thereof of an antibody selected from MBS11, MBS32, MBS22, MBS14, or MBS17, wherein the FVIII epitope is located in an A2 domain, an A3 domain, a C1 domain, a C2 domain, or any combinations thereof and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. The anti-FVIII antibody or antigen-binding molecule thereof can comprises six CDRs or variants thereof of an antibody selected from MBS11, MBS32, MBS22, MBS14, or MBS17.

In one embodiment, the invention includes an anti-FVIII antibody or antigen-binding molecule thereof which specifically binds to a FVIII epitope, which is an A2 region. For example, the anti-FVIII antibody or antigen-binding molecule thereof which specifically binds to a FVIII epitope can comprise:

(i) a variable heavy chain CDR-1 (VH-CDR1) sequence at least about 60G %, 70%, 80%, 90%, 95%, or 100% identical to a VH-CDR1 of an antibody selected from MBS11, MBS32, or MBS22;

(ii) a variable heavy chain CDR-2 (VH-CDR2) sequence at least about 60%, 70%, 80%, 90%, 95%, or 100% identical to a VH-CDR2 of an antibody selected from MBS11, MBS32, or MBS22;

(iii) a variable heavy chain CDR-3 (VH-CDR3) sequence at least about 60%, 70%, 80%, 90%, 95%, or 100% identical to a VH-CDR3 of an antibody selected from MBS11, MBS32, or MBS22;

(iv) a variable light chain CDR-1 (VL-CDR1) sequence at least about 60%, 70%, 80%, 90%, 95%, or 100% identical to a VL-CDR1 of an antibody selected from MBS11, MBS32, or MBS22;

(v) a variable light chain CDR-2 (VL-CDR2) sequence at least about 60%, 70%, 80%, 90%, 95%, or 100% identical to a VL-CDR2 of an antibody selected from MBS11, MBS32, or MBS22; and,

(vi) a variable light chain CDR-3 (VL-CDR3) sequence at least about 60%, 70%, 80%, 90%, 95%, or 100% identical to a VL-CDR3 of an antibody selected from MBS11, MBS32, or MBS22, wherein the FVIII epitope is located in an A2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, F VIIISELECT®, N772-10M, or OBT-0037A.

In another embodiment, an anti-FVIII antibody or antigen-binding molecule thereof which specifically binds to an A2 region comprises:

(i) the VH-CDR1 of an antibody selected from MBS11, MBS32, or MBS22;

(ii) the VH-CDR2 of an antibody selected from MBS11, MBS32, or MBS22;

(iii) the VH-CDR3 of an antibody selected from MBS11, MBS32, or MBS22;

(iv) the VL-CDR1 of an antibody selected from MBS11, MBS32, or MBS22;

(v) the VL-CDR2 of an antibody selected from MBS11, MBS32, or MBS22; and,

(vi) the VL-CDR3 of an antibody selected from MBS11, MBS32, or MBS22.

In another embodiment, the invention encompasses an anti-FVIII antibody or antigen-binding molecule thereof which specifically binds to an FVIII epitope, comprising a VH region, which comprises an amino acid sequence at least about 80%, 85%, 90%, 95%, or 100% identical to a VH of an antibody selected from MBS11, MBS32, or MBS22 and a VL region, which comprises an amino acid sequence at least about 80%, 85%, 90%, 95%, or 100% identical to a VL of an antibody selected from MBS11, MBS32, or MBS22, wherein the FVIII epitope is located in an A2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof comprises the VH of MBS11 and the VL of MBS11 (MBS11 antibody). In still other embodiments, an anti-FVIII antibody or antigen-binding molecule thereof comprises the VH of MBS32 and the VL of MBS32. In yet other embodiments, an anti-FVIII antibody or antigen-binding molecule thereof comprises the VH of MBS22 and the VL of MBS22.

In some embodiments, an anti-FVIII antibody or antigen-binding molecule thereof which specifically binds to a C1 domain of FVIII comprises:

(i) a variable heavy chain CDR-1 (VH-CDR1) sequence at least about 60%, 70%, 80%, 90%, 95%, or 100% identical to a VH-CDR1 of MBS14;

(ii) a variable heavy chain CDR-2 (VH-CDR2) sequence at least about 60%, 70%, 80%, 90%, 95%, or 100% identical to a VH-CDR2 of MBS14;

(iii) a variable heavy chain CDR-3 (VH-CDR3) sequence at least about 60%, 70%, 80%, 90%, 95%, or 100% identical to a VH-CDR3 of MBS14;

(iv) a variable light chain CDR-1 (VL-CDR1) sequence at least about 60%, 70%, 80%, 90%, 95%, or 100% identical to a VL-CDR1 of MBS14;

(v) a variable light chain CDR-2 (VL-CDR2) sequence at least about 60%, 70%, 80%, 90%, 95%, or 100% identical to a VL-CDR2 of MBS14; and,

(vi) a variable light chain CDR-3 (VL-CDR3) sequence at least about 60%, 70%, 80%, 90%, 95%, or 100% identical to a VL-CDR3 of MBS14 and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof comprises:

(i) the VH-CDR1 of MBS14;

(ii) the VH-CDR2 of MBS14;

(iii) the VH-CDR3 of MBS14;

(iv) the VL-CDR1 of MBS14;

(v) the VL-CDR2 of MBS14; and,

(vi) the VL-CDR3 of MBS14.

In certain embodiments, an anti-FVIII antibody or antigen-binding molecule thereof which specifically binds to a FVIII epitope, comprises a VH region, which comprises an amino acid sequence at least about 80%, 85%, 90%, 95%, or 100% identical to the VH of MBS14 and a VL region, which comprises an amino acid sequence at least about 80%, 85%, 90%, 95%, or 100% identical to the VL of MBS14, wherein the FVIII epitope is a C1 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. In other embodiments, an anti-FVIII antibody or antigen-binding molecule thereof comprises the VH of MBS14 and the VL of MBS14.

In certain embodiments, an anti-FVIII antibody or antigen-binding molecule thereof which specifically binds to a FVIII epitope comprises:

(i) a variable heavy chain CDR-1 (VH-CDR1) sequence at least about 60%, 70%, 80%/c, 90%, 95%, or 100% identical to a VH-CDR1 of MBS17;

(ii) a variable heavy chain CDR-2 (VH-CDR2) sequence at least about 60%, 70%, 80%, 900%, 95%, or 100% identical to a VH-CDR2 of MBS17;

(iii) a variable heavy chain CDR-3 (VH-CDR3) sequence at least about 60%, 70%, 80%, 90%, 95%, or 100% identical to a VH-CDR3 of MBS17;

(iv) a variable light chain CDR-1 (VL-CDR1) sequence at least about 60%, 70%, 80%, 90%, 95%, or 100% identical to a VL-CDR1 of MBS17;

(v) a variable light chain CDR-2 (VL-CDR2) sequence at least about 60%, 70%, 80%, 90%, 95%, or 100% identical to a VL-CDR2 of MBS17; and,

(vi) a variable light chain CDR-3 (VL-CDR3) sequence at least about 60%, 70%, 80%, 90%, 95%, or 100% identical to a VL-CDR3 of MBS17, wherein the FVIII epitope is located in a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof comprises:

(i) the VH-CDR1 of MBS17;

(ii) the VH-CDR2 of MBS17;

(iii) the VH-CDR3 of MBS17;

(iv) the VL-CDR1 of MBS17;

(v) the VL-CDR2 of MBS17; and,

(vi) the VL-CDR3 of MBS17.

In still other embodiments, an anti-FVIII antibody or antigen-binding molecule thereof which specifically binds to a FVIII epitope, comprises a VH region, which comprises an amino acid sequence at least about 80%, 85%, 90%, 95%, or 100% identical to the VH of MBS17 and a VL region, which comprises an amino acid sequence at least about 80%, 85%, 90%, 95%, or 100% identical to the VL of MBS17, wherein the FVIII epitope is located in a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In some embodiments, an anti-FVIII antibody or antigen-binding molecule thereof comprises the VH of MBSI7 and the VL of MBS17 (MBS17 antibody).

In other embodiments, an anti-FVII antibody or antigen-binding molecule thereof is a monoclonal antibody, a chimeric antibody, or a humanized antibody.

In some embodiments, the anti-FVIII antibody or antigen-binding molecule thereof comprises or consists of (a) a single chain Fv (“scFv”); (b) a diabody; (c) a minibody; (d) a polypeptide chain of an antibody; (e) F(ab′)₂; or (f) F(ab).

The present invention provides an anti-FVIII antibody or antigen-binding molecule thereof which specifically binds to a Factor VIII (“FVIII”) epitope, wherein the antibody or antigen-binding molecule thereof specifically binds to the same FVIII epitope as an antibody selected from GMA8023, GMA8024, GMA8045, GMA5G8, GMA8025, or GMA8026 and wherein the FVIII epitope is located in an A2 domain or a C2 domain, and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. The invention also provides an anti-FVIII antibody or antigen-binding molecule thereof which specifically binds to a Factor VIII (“FVIII”) epitope, wherein the anti-FVIII antibody or antigen-binding molecule thereof competitively inhibits FVIII binding by an antibody selected from GMA8023, GMA8024, GMA8045, GMA5G8, GMA8025, or GMA8026, wherein the FVIII epitope is located in an A2 domain or an C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. The invention also includes an anti-FVIII antibody or antigen-binding molecule thereof which specifically binds to a Factor VIII (“FVIII”) epitope, wherein the anti-FVIII antibody or antigen-binding molecule thereof comprises at least one, at least two, at least three, at least four, or at least five complementarity determining regions (CDR) or variants thereof of an antibody selected from GMA8023, GMA8024, GMA8045, GMA5G8, GMA8025, or GMA8026, wherein the FVIII epitope is located in an A2 domain or a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A. The anti-FVIII antibody or antigen-binding molecule thereof can comprises six CDRs or variants thereof of an antibody selected from MBS11, MBS32, MBS22, MBS14, or MBS17.

In one embodiment, the invention includes an anti-FVIII antibody or antigen-binding molecule thereof which specifically binds to a FVIII epitope, which is an A2 region. For example, the anti-FVIII antibody or antigen-binding molecule thereof which specifically binds to a FVIII epitope can comprise:

(i) a variable heavy chain CDR-1 (VH-CDR1) sequence at least about 60%, 70%, 80%, 90%, 95%, or 100% identical to a VH-CDR1 of an antibody selected from GMA8023 or GMA8024;

(ii) a variable heavy chain CDR-2 (VH-CDR2) sequence at least about 60%, 70%, 80%, 90%, 95%, or 100% identical to a VH-CDR2 of an antibody selected from GMA8023 or GMA8024;

(iii) a variable heavy chain CDR-3 (VH-CDR3) sequence at least about 60%, 70%, 80%, 90%, 95%, or 100% identical to a VH-CDR3 of an antibody selected from GMA8023 or GMA8024;

(iv) a variable light chain CDR-1 (VL-CDR1) sequence at least about 60%, 70%, 80%, 90%, 95%, or 100% identical to a VL-CDR1 of an antibody selected from GMA8023 or GMA8024;

(v) a variable light chain CDR-2 (VL-CDR2) sequence at least about 60%, 70%, 80%, 90%, 95%, or 100% identical to a VL-CDR2 of an antibody selected from GMA8023 or GMA8024; and,

(vi) a variable light chain CDR-3 (VL-CDR3) sequence at least about 60%, 70%, 80%, 90%, 95%, or 100% identical to a VL-CDR3 of an antibody selected from GMA8023 or GMA8024,

wherein the FVIII epitope is located in an A2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, F VIIISELECT®, N772-10M, or OBT-0037A.

In another embodiment, an anti-FVIII antibody or antigen-binding molecule thereof which specifically binds to an A2 region comprises:

(i) the VH-CDR1 of an antibody selected from GMA8023 or GMA8024;

(ii) the VH-CDR2 of an antibody selected from GMA8023 or GMA802422;

(iii) the VH-CDR3 of an antibody selected from GMA8023 or GMA8024;

(iv) the VL-CDR1 of an antibody selected from GMA8023 or GMA8024);

(v) the VL-CDR2 of an antibody selected from GMA8023 or GMA8024; and,

(vi) the VL-CDR3 of an antibody selected from GMA8023 or GMA8024.

In another embodiment, the invention encompasses an anti-FVIII antibody or antigen-binding molecule thereof which specifically binds to an FVIII epitope, comprising a VH region, which comprises an amino acid sequence at least about 80%, 85%, 90%, 95%, or 100% identical to a VH of an antibody selected from GMA8023 or GMA8024 and a VL region, which comprises an amino acid sequence at least about 80%, 85%, 90%, 95%, or 100% identical to a VL of an antibody selected from GMA8023 or GMA8024, wherein the FVIII epitope is located in an A2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof comprises the VH of GMA8023 and the VL of GMA8023. In still other embodiments, an anti-FVIII antibody or antigen-binding molecule thereof comprises the VH of GMA8024 and the VL of GMA8024.

In certain embodiments, an anti-FVIII antibody or antigen-binding molecule thereof which specifically binds to a FVIII epitope comprises:

(i) a variable heavy chain CDR-1 (VH-CDR1) sequence at least about 60%, 70%, 80%, 90%, 95%, or 100% identical to a VH-CDR1 of GMA8045, GMA5G8, GMA8025, or GMA8026;

(ii) a variable heavy chain CDR-2 (VH-CDR2) sequence at least about 60%, 70%, 800%, 90%, 95%, or 100% identical to a VH-CDR2 of GMA8045, GMA5G8, GMA8025, or GMA8026;

(iii) a variable heavy chain CDR-3 (VH-CDR3) sequence at least about 60%, 70%, 80%, 90%, 95%, or 100% identical to a VH-CDR3 of GMA8045, GMA5G8, GMA8025, or GMA8026;

(iv) a variable light chain CDR-1 (VL-CDR1) sequence at least about 60%, 70%, 80%, 90%, 95%, or 100% identical to a VL-CDR1 of GMA8045, GMA5G8, GMA8025, or GMA8026;

(v) a variable light chain CDR-2 (VL-CDR2) sequence at least about 60%, 70%, 80%, 90%, 95%, or 100% identical to a VL-CDR2 of GMA8045, GMA5G8, GMA8025, or GMA8026; and,

(vi) a variable light chain CDR-3 (VL-CDR3) sequence at least about 60%, 70%, 80%, 90%, 95%, or 100% identical to a VL-CDR3 of GMA8045, GMA5G8, GMA8025, or GMA8026,

wherein the FVIII epitope is located in a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In other embodiments, an anti-FVIII antibody or antigen-binding molecule thereof comprises:

(i) the VH-CDR1 of GMA8045, GMA5G8, GMA8025, or GMA8026;

(ii) the VH-CDR2 of GMA8045, GMA5G8, GMA8025, or GMA8026;

(iii) the VH-CDR3 of GMA8045, GMA5G8, GMA8025, or GMA8026;

(iv) the VL-CDR1 of GMA8045, GMA5G8, GMA8025, or GMA8026;

(v) the VL-CDR2 of GMA8045, GMA5G8, GMA8025, or GMA8026; and,

(vi) the VL-CDR3 of GMA8045, GMA5G8, GMA8025, or GMA8026.

In still other embodiments, an anti-FVIII antibody or antigen-binding molecule thereof which specifically binds to a FVIII epitope, comprises a VH region, which comprises an amino acid sequence at least about 80%, 85%, 90%, 95%, or 100% identical to the VH of GMA8045, GMA5G8, GMA8025, or GMA8026 and a VL region, which comprises an amino acid sequence at least about 80%, 85%, 90%, 95%, or 100% identical to the VL of GMA8045, GMA5G8, GMA8025, or GMA8026, wherein the FVIII epitope is located in a C2 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

In some embodiments, an anti-FVIII antibody or antigen-binding molecule thereof comprises the VH of GMA8045, GMA5G8, GMA8025, or GMA8026 and the VL of GMA8045, GMA5G8, GMA8025, or GMA8026.

In other embodiments, an anti-FVIII antibody or antigen-binding molecule thereof is a monoclonal antibody, a chimeric antibody, or a humanized antibody. In some embodiments, the anti-FVIII antibody or antigen-binding molecule thereof comprises or consists of (a) a single chain Fv (“scFv”); (b) a diabody; (c) a minibody; (d) a polypeptide chain of an antibody; (e) F(ab′)₂; or (f) F(ab).

### II.B. Methods of Making

The present disclosure also provides a nucleic acid molecule or a set of nucleic acid molecules encoding an anti-FVIII antibody or antigen-binding molecule thereof (e.g., MBS11, MBS32, MBS22, MBS14, GMA8023, GMA8024, GMA8045, GMA5G8, GMA8025, GMA8026, or MBS17) or a complement thereof.

Also provided is a vector or a set of vectors comprising such nucleic acid molecules or the set of the nucleic acid molecules or a complement thereof or a host cell comprising the vector.

The instant disclosure also provides a method for producing an anti-FVIII antibody or antigen-binding molecule thereof, such method comprising culturing the host cell disclosed herein and recovering the antibody or antigen-binding molecule thereof from the culture medium.

A variety of methods are available for recombinantly producing an anti-FVIII antibody or antigen-binding molecule thereof disclosed herein. It will be understood that because of the degeneracy of the code, a variety of nucleic acid sequences will encode the amino acid sequence of the polypeptide. The desired polynucleotide can be produced by de novo solid-phase DNA synthesis or by PCR mutagenesis of an earlier prepared polynucleotide.

Oligonucleotide-mediated mutagenesis is one method for preparing a substitution, in-frame insertion, or alteration (e.g., altered codon) to introduce a codon encoding an amino acid substitution (e.g., into an anti-FVIII antibody variant). For example, the starting polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide encoding an anti-FVIII antibody or antigen-binding molecule thereof disclosed herein.

For recombinant production, a polynucleotide sequence encoding a polypeptide (e.g., an anti-FVIII antibody or antigen-binding molecule thereof disclosed herein) is inserted into an appropriate expression vehicle, i.e., a vector which contains the necessary elements for the transcription and translation of the inserted coding sequence, or in the case of an RNA viral vector, the necessary elements for replication and translation.

The nucleic acid encoding the polypeptide (e.g., an anti-FVIII antibody or antigen-binding molecule thereof disclosed herein) is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO J. 1:841). A variety of host-expression vector systems can be utilized to express the polypeptides described herein (e.g., an anti-FVIII antibody or antigen-binding molecule thereof disclosed herein) in eukaryotic cells. In one embodiment, the eukaryotic cell is an animal cell, including mammalian cells (e.g., 293 cells, PerC6, CHO, BHK, Cos, HeLa cells). When the polypeptide is expressed in a eukaryotic cell, the DNA encoding the polypeptide (e.g., an anti-FVIII antibody or antigen-binding molecule thereof disclosed herein) can also code for a signal sequence that will permit the polypeptide to be secreted. One skilled in the art will understand that while the polypeptide is translated, the signal sequence is cleaved by the cell to form the mature antibody sequence. Various signal sequences are known in the art, e.g., native FVII signal sequence, native FIX signal sequence, native FX signal sequence, and the mouse IgK light chain signal sequence. Alternatively, where a signal sequence is not included, the polypeptide (e.g., an anti-FVIII antibody or antigen-binding molecule thereof disclosed herein) can be recovered by lysing the cells.

The expression vectors can encode for tags that permit for easy purification or identification of the recombinantly produced polypeptide. Examples include, but are not limited to, vector pUR278 (Ruther et al. 1983, EMBO J. 2:1791) in which the polypeptide (e.g., an anti-FVIII antibody or antigen-binding molecule thereof disclosed herein) coding sequence can be ligated into the vector in frame with the lac z coding region so that a polypeptide is produced; pGEX vectors can be used to express proteins with a glutathione S-transferase (GST) tag. These proteins are usually soluble and can easily be purified from cells by adsorption to glutathione-agarose beads followed by elution in the presence of free glutathione. The vectors include cleavage sites, e.g., for PreCission Protease (Pharmacia, Peapack, N. J.) for easy removal of the tag after purification.

For the purposes of this invention, numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation.

An examplary expression vector is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other embodiments, polypeptides of the invention (e.g., an anti-FVIII antibody or antigen-binding molecule thereof disclosed herein) can be expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is also incorporated herein. Those skilled in the art will appreciate that such expression systems can be used to effectively produce the full range of polypeptides disclosed in the instant application.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), flow cytometry, immunohistochemistry, and the like.

As used herein, the term “transformation” refers in a broad sense to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of polypeptide unless it is clearly specified otherwise. In other words, recovery of polypeptide from the “cells” can mean either from spun down whole cells, or from the cell culture containing both the medium and the suspended cells.

In one embodiment, a host cell endogenously expresses an enzyme (or the enzymes) necessary to cleave the heavy chain FVIII from the light chain FVIII (e.g., if such a linker is present and contains intracellular processing site(s)) during processing to form the mature two chain polypeptides. In another embodiment of the invention, a host cell is transformed to express one or more enzymes which are exogenous to the cell such that processing of FVIII occurs or is improved.

In one embodiment an enzyme which can be endogenously or exogenously expressed by a cell is a member of the furin family of enzymes. Complete cDNA and amino acid sequences of human furin (i.e., PACE) were published in 1990. Van den Ouweland A M et al. (1990) Nucleic Acids Res. 18:664; Erratum in: Nucleic Acids Res. 18:1332 (1990). U.S. Pat. No. 5,460,950, issued to Barr et al., describes recombinant PACE and the coexpression of PACE with a substrate precursor polypeptide of a heterologous protein to improve expression of active, mature heterologous protein. U.S. Pat. No. 5,935,815, likewise describes recombinant human furin (i.e., PACE) and the coexpression of furin with a substrate precursor polypeptide of a heterologous protein to improve expression of active, mature heterologous protein. Other family members in the mammalian furin/subtilisin/Kex2p-like proprotein convertase (PC) family in addition to PACE are reported to include PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). While these various members share certain conserved overall structural features, they differ in their tissue distribution, subcellular localization, cleavage specificities, and preferred substrates. For a review, see Nakayama K (1997) Biochem J. 327:625-35. Similar to PACE, these proprotein convertases generally include, beginning from the amino terminus, a signal peptide, a propeptide (that can be autocatalytically cleaved), a subtilisin-like catalytic domain characterized by Asp, His, Ser, and Asn/Asp residues, and a Homo B domain that is also essential for catalytic activity and characterized by an Arg-Gly-Asp (RGD) sequence. PACE, PACE4, and PC5 also include a Cys-rich domain, the function of which is unknown. In addition, PC5 has isoforms with and without a transmembrane domain; these different isoforms are known as PC5B and PC5A, respectively. Comparison between the amino acid sequence of the catalytic domain of PACE and the amino acid sequences of the catalytic domains of other members of this family of proprotein convertases reveals the following degrees of identity: 70 percent for PC4; 65 percent for PACE4 and PC5; 61 percent for PC1/PC3; 54 percent for PC2; and 51 percent for LPC/PC7/PC8/SPC7. Nakayama K (1997) Biochem J. 327:625-35.

PACE and PACE4 have been reported to have partially overlapping but distinct substrates. In particular, PACE4, in striking contrast to PACE, has been reported to be incapable of processing (i.e., cleaving) the heavy chain FVIII and the light chain FVIII. See WO 2012/006623 A2, published Dec. 1, 2012. U.S. Pat. No. 5,840,529, discloses nucleotide and amino acid sequences for human PC7 and the notable ability of PC7, as compared to other PC family members, to cleave HIV gp160 to gp120 and gp41.

Nucleotide and amino acid sequences of rodent PC5 were first described as PC5 by Lusson et al. (1993) Proc Natl Acad Sci USA 90:6691-5 and as PC6 by Nakagawa et al. (1993) J Biochem (Tokyo) 113:132-5. U.S. Pat. No. 6,380,171 discloses nucleotide and amino acid sequences for human PC5A, the isoform without the transmembrane domain. The sequences of these enzymes and method of cloning them are known in the art.

Genes encoding the polypeptides of the invention (e.g., an anti-FVIII antibody or antigen-binding molecule thereof disclosed herein) can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

In addition to prokaryates, eukaryotic microbes can also be used. Saccharomyces cerevisiae, or common baker's yeast, is the most commonly used among eukaryotic microorganisms although a number of other strains are commonly available.

For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

Alternatively, polypeptide-coding nucleotide sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein. An affinity tag sequence (e.g. a His(6) tag) can optionally be attached or included within the polypeptide sequence to facilitate downstream purification.

Once expressed, the chimeric molecules can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)) and see specifically the methods used in the instant Examples. Substantially pure proteins of at least about 90 to 95% homogeneity are preferred, and 98 to 99% or more homogeneity most preferred, for pharmaceutical uses.

### III. Methods of Using Anti-FVIII Antibodies

The anti-FVIII antibodies disclosed herein can be used in various methods depending their specificity and affinity to FVIII. In one aspect, some anti-FVIII antibodies are identified as the antibodies that are capable of releasing FVIII in buffers while maintaining FVIII activity after releasing FVIII. These anti-FVIII antibodies can be used for immunoaffinity purification. In another aspect, anti-FVIII antibodies that compete with VWF for the binding to FVIII can be used to reduce, prevent, or inhibit the binding of VWF with FVIII. In other aspects, anti-FVIII antibodies compete their binding to FVIII with one or more FVIII-binding molecules or one or more FVIII inhibitors. These antibodies can be used to identify the FVIII-binding molecules or FVIII inhibitors or identify the binding site of the FVIII-binding molecules or FVIII inhibitors. Exemplary uses of the anti-FVIII antibodies are provided below.

### III.B. Antibodies Useful for Immunoaffinity Purification

Some anti-FVIII antibodies can change, reduce, or ablate the FVIII activity after binding to a FVIII protein. Other anti-FVIII antibodies can bind to FVIII tightly and do not release FVIII commonly used buffers. Still other anti-FVIII antibodies are capable of releasing FVIII only in buffers, such as those with pH values well ouside the neutral range, i.e., greater than approximately pH 9 or less than approximately pH 4, that will change, reduce or ablate activity of FVIII. However, some anti-FVIII antibodies are identified as anti-FVIII antibodies that are capable of releasing FVIII in a buffer that maintains FVIII activity. Such antibodies can be used for immunoaffinity purification of a FVIII protein, as the antibodies would release FVIII in elutions buffers without affecting the activity of FVIII.

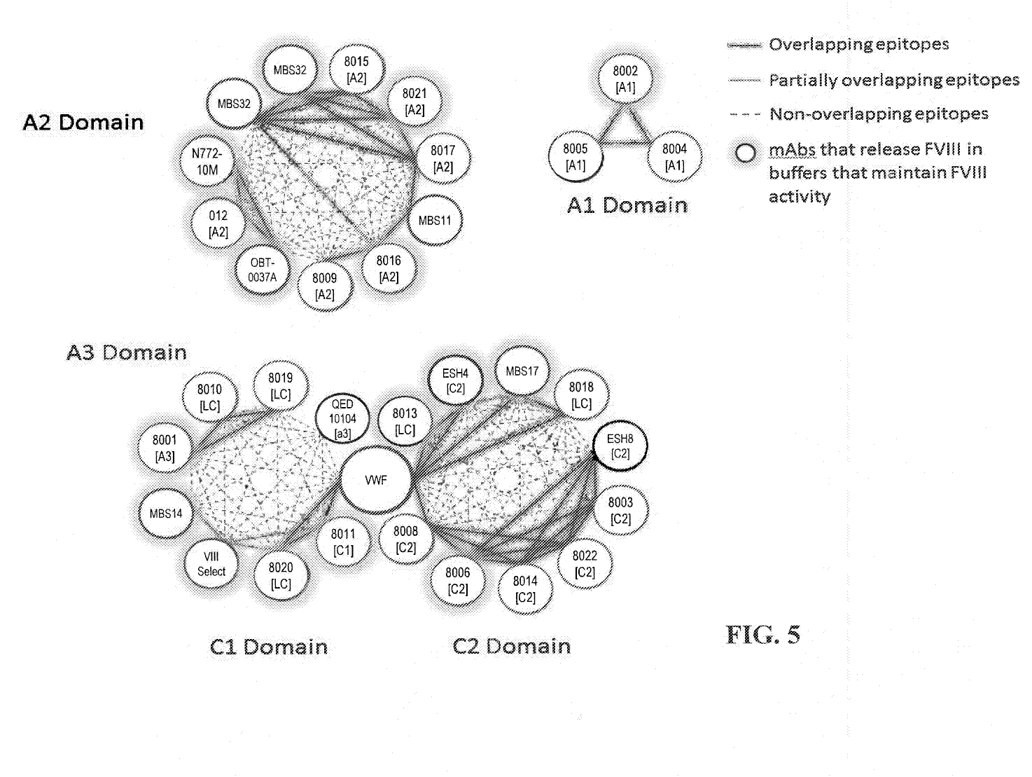

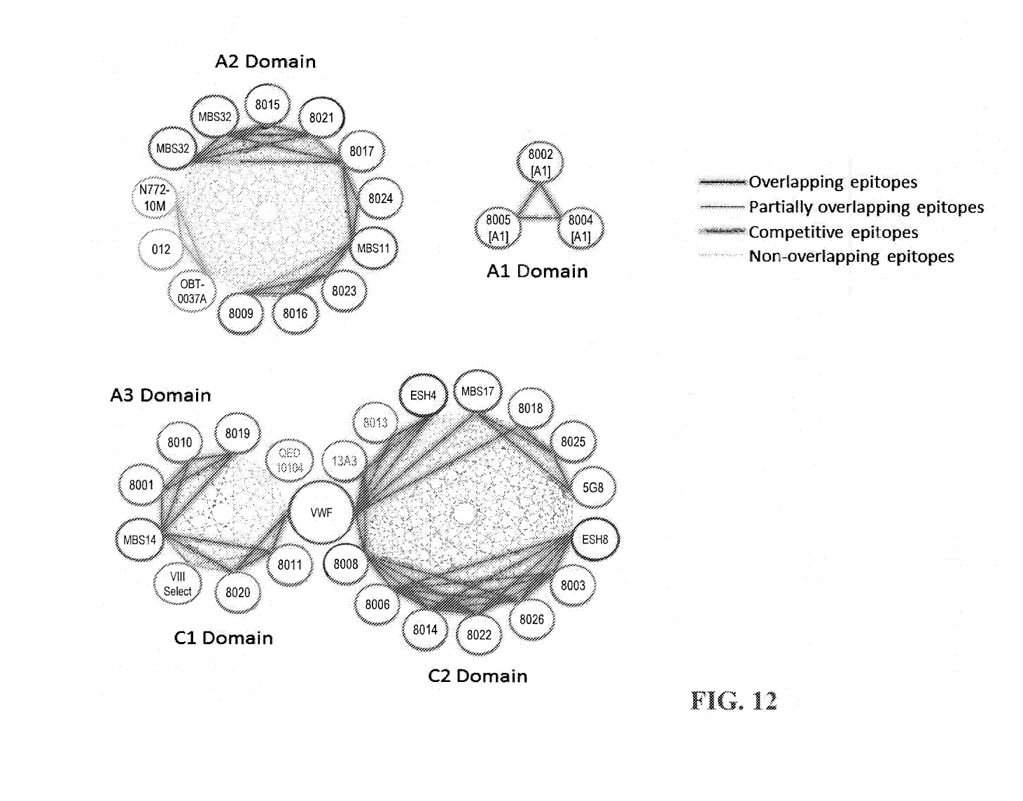

As FIG. 5 and FIG. 12 show, the anti-FVIII antibodies that are capable of releasing FVIII in a buffer while maintaining the FVIII activity include, but are not limited to, GMA8002, GMA8021, GMA8016, MBS32, GMA012, N772-10M, OBT-0037A, GMA8001, MBS14, GMA8020, GMA8011, GMA8013, GMA8023, GMA8024, GMA5G8, GMA5E8, GMA8026, or GMA8006. In one embodiment, the invention includes a method of purifying a FVIII protein comprising contacting a GMA8002 antibody, a GMA8021 antibody, a GMA8016 antibody, a MBS32 antibody, a GMA012 antibody, a N772-10M antibody, a OBT-0037A antibody, a GMA8001 antibody, a MBS14 antibody, a GMA8020 antibody, a GMA8011 antibody, a GMA8013 antibody, a GMA8006 antibody, a GMA8023 antibody, a GMA8024 antibody, a GMA5G8 antibody, a GMA5E8 antibody, a GMA8026 antibody, or an antigen-binding molecule thereof with the FVIII protein. The FVIII protein can then be eluted by a buffer and be isolated. The purified/isolated FVIII protein can then be formulated and administered to a subject in need thereof.

In another embodiment, the anti-FVIII antibodies that are useful for purifying a FVIII protein are A1 domain-specific antibodies. For example, a method of purifying a FVIII protein can comprise contacting the FVIII protein with an anti-FVIII antibody or antigen-binding molecule thereof that binds to an A1 domain. In a particular embodiment, an example of anti-FVIII antibodies or an antigen-binding molecule thereof that bind to an A1 domain of FVIII is a GMA8002 antibody, an antigen-binding molecule thereof, or a variant or derivative thereof.

In other embodiments, the anti-FVIII antibodies that are useful for purifying a FVIII protein are A2 domain-specific antibodies. For example, a method of purifying a FVIII protein can comprise contacting the FVIII protein with an anti-FVIII antibody or antigen-binding molecule thereof that binds to an A2 domain. Non-limiting examples of the anti-FVIII antibodies or an antigen-binding molecule thereof that bind to an A2 domain of FVIII can include a GMA8021 antibody, a GMA8016 antibody, a MBS32 antibody, a N772-10M antibody, a GMA012 antibody, a GMA8023 antibody, a GMA8024 antibody, or an OBT-0037A antibody, an antigen-binding molecule thereof, or a variant or derivative thereof. In a particular embodiment, an A2 domain-specific antibody is a MBS32 antibody.

In still other embodiments, the anti-FVIII antibodies that are useful for purifying a FVIII protein are A3 domain-specific antibodies. For example, a method of purifying a FVIII protein can comprise contacting the FVIII protein with an anti-FVIII antibody or antigen-binding molecule thereof that binds to an A3 domain. Non-limiting examples of anti-FVIII antibodies or one or more antigen-binding molecule thereof that bind to an A3 domain of FVIII are a GMA8001 antibody or a MBS14 antibody, an antigen-binding molecule thereof, or a variant or derivative thereof.

In yet other embodiments, the anti-FVIII antibodies that are useful for purifying a FVIII protein are A3 domain-specific antibodies. For example, a method of purifying a FVIII protein can comprise contacting the FVIII protein with an anti-FVIII antibody or antigen-binding molecule thereof that binds to an A3 domain. In a particular embodiment, examples of anti-FVIII antibodies or an antigen-binding molecule thereof that bind to an A3 domain of FVIII include, but are not limited to, a GMA8001 antibody, a MBS14 antibody, an antigen-binding molecule thereof, or a variant or derivative thereof.

In some embodiments, the anti-FVIII antibodies that are useful for purifying a FVIII protein are C1 domain-specific antibodies. For example, a method of purifying a FVIII protein can comprise contacting the FVIII protein with an anti-FVIII antibody or antigen-binding molecule thereof that binds to a C1 domain. In a particular embodiment, an example of anti-FVIII antibodies or an antigen-binding molecule thereof that bind to a C1 domain of FVIII is a GMA8011 antibody, an antigen-binding molecule thereof, or a variant or derivative thereof.

In certain embodiments, the anti-FVIII antibodies that are useful for purifying a FVIII protein are C2 domain-specific antibodies. For example, a method of purifying a FVIII protein can comprise contacting the FVIII protein with an anti-FVIII antibody or antigen-binding molecule thereof that binds to a C2 domain. In a particular embodiment, an example of anti-FVIII antibodies or an antigen-binding molecule thereof that bind to a C2 domain of FVIII is a GMA8006 antibody, a GMA5G8 antibody, a GMA5E8 antibody, or a GMA8026 antibody, an antigen-binding molecule thereof, or a variant or derivative thereof.

In some embodiments, the anti-FVIII antibodies that are useful for purifying a FVIII protein are light chain-specific antibodies. For example, a method of purifying a FVIII protein can comprise contacting the FVIII protein with an anti-FVIII antibody or antigen-binding molecule thereof that binds to a light chain. In a particular embodiment, non-limiting examples of anti-FVIII antibodies or an antigen-binding molecule thereof that bind to a light chain of FVIII are a GMA8020 antibody, a GMA5E8 antibody, a GMA8013 antibody, an antigen-binding molecule thereof, or a variant or derivative thereof.

In certain embodiments, the elution buffer for the anti-FVIII antibodies or antigen-binding molecule thereof comprises propylene glycol, or arginine, or both. In one embodiment, the elution buffer comprises at least about 30% (v/v), at least about 40% (v/v), at least about 45% (v/v), at least about 50% (v/v), at least about 55% (v/v), at least about 60% (v/v), at least about 65% (v/v), at least about 70% (v/v), or at least about 75% (v/v) propylene glycol. In another embodiment, the elution buffer comprises at least about 0.5M, at least about 0.6M, at least about 0.7M, at least about 0.8M, at least about 0.9M, at least about 1.0M, at least about 1.1M, at least about 1.2M, or at least about 1.3M. of arginine. In other embodiments, the buffer comprises about 30% (v/v) to about 80% (v/v) propylene glycol, about 40% (v/v) to about 70% (v/v) propylene glycol, about 40% (v/v) to about 60% (v/v) propylene glycol, or about 40% (v/v) to about 50% (v/v) propylene glycol. In some embodiments, the buffer comprises about 0.5M to about 2M of arginine. In certain embodiments, arginine can be L-arginine or arginine homologs. Examples of the arginine homologs include, but are not limited to, 2-amino-3-guanidinopropionic acid, 2-amino-4-guanidino-butyric acid and 2-amino-6-guanidinocaproic acid.

In some embodiments, the buffer comprises propylene glycol, arginine, and. Histidine. In other embodiments, the buffer comprises propylene glycol, arginine, and CaCl₂. In certain embodiments, the buffer comprises propylene glycol, arginine, histidine, CaCl₂, and Tween-20. For example, the buffer can comprise about 10 mM to about 100 mM of histidine, about 0.1M to about 1.5M of arginine, about 10 mM to about 100 mM of CaCl2, about 30% (v/v/) to about 70% (v/v/) of propylene glycol, and about 0.01% to about 0.1% of Tween-20. In a specific embodiment, the buffer comprises about 50 mM histidine, about 0.9M arginine, about 50 mM CaCl2, about 45% (v/v) propylene glycol, and about 0.05% Tween-20 at pH 7.2.

The present invention also includes a method of purifying a FVIII protein, comprising contacting an anti-FVIII antibody or antigen-binding molecule thereof, which specifically binds to a FVIII epitope, with the FVIII protein and eluting the FVIII protein in a high ionic buffer, wherein the anti-FVIII antibody or antigen-binding molecule thereof comprises:

(i) a variable heavy chain CDR-1 (VH-CDR1) sequence at least about 60%, 70%, 80%, 90%, 95%, or 100% identical to a VH-CDR1 of an antibody selected from ESH4, GMA8013, MBS17, GMA5G8, GMA5E8, GMA8025, or OBT0037A,

(ii) a variable heavy chain CDR-2 (VH-CDR2) sequence at least about 60%, 70%, 80%, 90%, 95%, or 100% identical to a VH-CDR2 of an antibody selected from ESH4, GMA8013, MBS17, GMA5G8, GMA5E8, GMA8025, or OBT0037A;

(iii) a variable heavy chain CDR-3 (VH-CDR3) sequence at least about 60%, 70%, 80%, 90%, 95%, or 100% identical to a VH-CDR3 of an antibody selected from ESH4, GMA8013, MBS17, GMA5G8, GMA5E8, GMA8025, or OBT0037A;

(iv) a variable light chain CDR-1 (VL-CDR1) sequence at least about 60%, 70%, 80%, 90%, 95%, or 100% identical to a VL-CDR1 of an antibody selected from ESH4, GMA8013, MBS17, GMA5G8, GMA5E8, GMA8025, or OBT0037A;

(v) a variable light chain CDR-2 (VL-CDR2) sequence at least about 60%, 70%, 80%, 90%, 95%, or 100% identical to a VL-CDR2 of an antibody selected from ESH4, GMA8013, MBS17, GMA5G8, GMA5E8, GMA8025, or OBT0037A; and,

(vi) a variable light chain CDR-3 (VL-CDR3) sequence at least about 60%, 70%, 80%, 90%, 95%, or 100% identical to a VL-CDR3 of an antibody selected from ESH4, GMA8013, MBS17, GMA5G8, GMA5E8, GMA8025, or OBT0037A,

wherein the FVIII epitope is located in a light chain, an A2 region, a C2 domain, or any combinations thereof.

In one embodiment, the anti-FVIII antibody or antigen-binding molecule thereof comprises a VH region and a VL region, wherein the VH region comprises an amino acid sequence at least 60%, at least 70%, at least 80%, at least 90%, at least 95%, or at least 100% identical to a VH region of an antibody selected from ESH4, GMA8013, MBS17, GMA5G8, GMA5E8, GMA8025, or OBT0037A and wherein the VL region comprises an amino acid sequence at least 60%, at least 70%, at least 80%, at least 90%, at least 95%, or at least 100% identical to a VL region of an antibody selected from ESH4, GMA8013, MBS17, GMA5G8, GMA5E8, GMA8025, or OBT0037A.

In another embodiment, the FVIII epitope to which the anti-FVIII antibody or antigen-binding molecule thereof is located in the C2 domain, wherein the anti-FVIII antibody or antigen-binding molecule thereof comprises a VH region and a VL region, wherein the VH region comprises the VH of ESH4 and the VL region comprises the VL of ESH4. The anti-FVIII antibody or antigen-binding molecule thereof that binds to the C2 domain can comprise a VH region and a VL region, wherein the VH region comprises the VH of an antibody selected from MSB17, and the VL region comprises the VL of an antibody selected from MBS17.

In other embodiments, the FVIII epitope to which the anti-FVIII antibody or antigen-binding molecule thereof is located in the light chain, wherein the anti-FVIII antibody or antigen-binding molecule thereof comprises a VH region and a VL region, wherein the VH region comprises the VH of an antibody selected from GMA8013, and the VL region comprises the VL of an antibody selected from GMA8013.

In some embodiments, the FVIII epitope to which the anti-FVIII antibody or antigen-binding molecule thereof binds to is located in the A2 domain, wherein the anti-FVIII antibody or antigen-binding molecule thereof comprises a VH region and a VL region, wherein the VH region comprises the VH of OBT0037A and the VL region comprises the VL of OBT0037A.

In certain aspects, the anti-FVIII antibody or antigen-binding molecule thereof binds to the FVIII protein at a dissociation constant (KD) lower than about 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, 0.005 nM, or 0.001 nM.

In some aspects, the high ionic buffer comprises NaCl, CaCl₂, Tris-HCl, or any combinations thereof. In one embodiment, the high ionic buffer comprises at least about 5 nM, at least about 10 nM, at least about 15 nM, at least about 20 nM, at least about 25 nM, at least about 30 nM, at least about 40 nM, at least about 50 nM, at least about 60 nM, at least about 70 nM, at least about 80 nM Tris-HCl. In another embodiment, the high ionic buffer comprises at least about 0.1M, at least about 0.2M, at least about 0.3M, at least about 0.4M, at least about 0.5M, at least about 0.6M, at least about 0.7M, at least about 0.8M, at least about 0.9M, at least about 1.0M, at least about 1.1M, at least about 1.2M, at least about 1.3M, at least about 1.4M, or at least about 1.5M NaCl. In certain aspects, the high ionic buffer comprises at least about 0.1M CaCl₂, at least about 0.15M, at least about 0.2M, at least about 0.25M, at least about 0.3M, at least about 0.35M, at least about 0.4M, at least about 0.45M, at least about 0.5M, at least about 0.6M, at least about 0.65M, at least about 0.7M, at least about 0.75M, at least about 0.8M, at least about 0.85M, at least about 0.9M, at least about 0.95M, or at least about 1.0M CaCl₂. In a specific example, the high ionic buffer comprises about 20 nM Tris-HCl, about 0.6M NaCl, about 0.35M CaCl₂ at pH 7.2.

### III.A. Antibodies Binding to VWF

Some anti-FVIII antibodies can compete with VWF for the binding to FVIII. In one embodiment, such antibodies can be used to reduce, prevent, or inhibit FVIII binding to VWF by blocking a VWF binding site on FVIII. Those anti-FVIII antibodies that are capable of binding to a VWF binding site include, but are not limited to, GMA8018, MBS17, ESH4, GMA8013, GMA8008, GMA8011, GMA8045, or GMA8020. In one aspect, such antibodies can be used to design a FVIII protein that does not bind to VWF or that does bind to VWF.

Therefore, in one embodiment, the invention includes a method of reducing, preventing, or inhibiting the binding of a FVIII protein with VWF comprising contacting the FVIII protein with an anti-FVIII antibody or antigen-binding molecule thereof that is capable of blocking a VWF binding site of FVIII, e.g., a GMA8018 antibody, a MBS17 antibody, a ESH4 antibody, a GMA8013 antibody, a GMA8008 antibody, a GMA8011 antibody, a GMA8045 antibody, or a GMA8020 antibody.

In another embodiment, a method of reducing, preventing, or inhibiting the binding of a FVIII protein to VWF comprises contacting the FVIII protein with an anti-FVIII antibody or antigen-binding molecule thereof, which binds to VWF, in a medium that does not contain VWF or in a medium that contains VWF. In a medium that does not contain any VWF, the anti-FVIII antibody or antigen-binding molecule thereof can bind to the FVIII protein at a VWF binding site without any competition from VWF. When VWF is added to the mixture of the anti-FVIII antibody or antigen-binding molecule thereof and the FVIII protein, VWF cannot bind to the FVIII protein due to the anti-FVIII antibody or antigen-binding molecule thereof. Thus, the method can further comprise measuring the binding of the VWF in the mixture of the anti-FVIII antibody or antigen-binding molecule thereof, the FVIII protein, and VWF.

In one aspect, this method can be used to design a FVIII variant that does not bind to VWF. For example, a FVIII protein, while being designed, can be tested to measure its ability (or inability) to bind to VWF using the anti-FVIII antibody or antigen-binding molecule thereof.

In another aspect, this method can be used to design or select a FVIII variant that is capable of binding to VWF. For example, a FVIII protein containing an insertion of a heterologous moiety at around the VWF binding site and still having the ability to bind to VWF can be identified.

In some aspects, the method comprises contacting a FVIII protein with an anti-FVIII antibody or antigen-binding molecule thereof in the presence of VWF; thereby the anti-FVIII antibody or antigen-binding molecule thereof competes with VWF for the binding to the FVIII protein. The method further comprises the binding of the FVIII protein to the anti-FVIII antibody or antigen-binding molecule thereof.

In other aspects, the invention includes a method of identifying a FVIII protein that does not bind to VWF comprising contacting an anti-FVIII antibody or antigen-binding molecule thereof with the FVIII protein, measuring the binding of the anti-FVIII antibody or antigen-binding molecule thereof with the FVIII protein, and isolating the FVIII protein that does not bind to the anti-FVIII antibody or antigen-binding molecule thereof.

In certain aspects, the VWF that binds to the FVIII protein comprises full-length mature VWF or a VWF fragment. In other aspects, the VWF is endogenous VWF. In some aspects, the FVIII protein identified and purified/isolated from the methods has a half-life longer than the half-life of a protein consisting of full-length wild-type FVIII. In other aspects, the methods further comprise administering the isolated FVIII protein to a subject in need thereof. The subject can have hemophilia A.

### III.C. Antibodies Competing with FVIII-Binding Molecule or FVIII Inhibitor

Each FVIII antibody can bind to one or more domains of FVIII. Identifying the specific epitope can lead to identification of a FVIII-binding molecule or a FVIII inhibitor that binds to FVIII. The epitope domains of the anti-FVIII antibodies and the affinities are provided in Table 1.

TABLE 1
Binding Epitope and Affinity of Anti-FVIII Antibodies

| | | BDD | | | | |
|---|---|---|---|---|---|---|
| | | rFVIII | | rFVIIIFc | | |
| | | Mean | | Mean | | Instru- |
| | Antibody | K_{D} (M) | SD (M) | K_{D} (M) | SD (M) | ment |
| A1 | GMA 8005 | 4.63E−10 | 9.91E−11 | 6.55E−10 | 1.89E−10 | ProteOn |
| A1 | GMA 8002 | 2.30E−10 | 7.27E−11 | 4.57E−10 | 1.83E−10 | Biacore |
| A2 | GMA 012 | 3.25E−10 | 1.16E−10 | 4.74E−10 | 9.55E−11 | ProteOn |
| A2 | GMA 8809 | 5.51E−11 | 1.14E−11 | 6.27E−11 | 8.63E−12 | ProteOn |
| A2 | GMA 8016 | 1.90E−10 | 5.58E−11 | 3.68E−10 | 5.49E−11 | ProteOn |
| A2 | GMA 8017 | 3.86E−10 | 4.27E−11 | 3.82E−10 | 9.31E−11 | ProteOn |
| A2 | GMA 8023 | | | | | |
| A2 | GMA 8024 | | | | | |
| A2 | MBS11 | 1.47E−09 | 3.50E−10 | 1.48E−09 | 2.03E−10 | ProteOn |
| A2 | MBS32 | 4.10E−10 | 4.80E−11 | 3.62E−10 | 1.27E−10 | ProteOn |
| A2 | MBS22 | 1.11E−11 | 5.18E−12 | 1.13E−11 | 4.66E−12 | ProteOn |
| A3 | GMA 8001 | 8.20E−10 | 2.69E−10 | 1.03E−09 | 1.89E−10 | ProteOn |
| A3 | GMA 8010 | 1.07E−10 | 1.30E−11 | 1.26E−10 | 1.08E−11 | ProteOn |
| A3 | GMA 8019 | 3.82E−11 | 5.30E−12 | 4.70E−11 | 1.03E−11 | ProteOn |
| C1 | GMA 8011 | 9.21E−11 | 8.02E−12 | 8.73E−11 | 6.00E−12 | ProteOn |
| C1 | GMA 8020 | 7.31E−11 | 3.58E−12 | 6.83E−11 | 5.21E−12 | ProteOn |
| C1 | MBS14 | 7.62E−10 | 1.43E−10 | 1.35E−09 | 1.40E−10 | ProteOn |
| C2 | ESH4 | 2.48E−10 | 2.18E−11 | 2.77E−10 | 2.81E−11 | ProteOn |
| C2 | ESH8 | 1.04E−10 | 7.31E−12 | 1.42E−10 | 2.31E−12 | ProteOn |
| C2 | GMA 8003 | 3.94E−10 | 2.00E−11 | 4.41E−10 | 4.38E−11 | ProteOn |
| C2 | GMA 8006 | 1.58E−10 | 1.56E−11 | 2.19E−10 | 4.78E−11 | ProteOn |
| C2 | GMA 8008 | 7.14E−11 | 6.56E−12 | 7.33E−11 | 1.58E−11 | ProteOn |
| C2 | GMA 8013 | 1.02E−10 | 8.39E−12 | 1.27E−10 | 2.16E−11 | ProteOn |
| C2 | GMA 8014 | 1.18E−10 | 8.08E−12 | 1.18E−10 | 1.01E−11 | ProteOn |
| C2 | GMA 8045 | 8.07E−11 | 1.14E−11 | 7.97E−11 | 2.29E−11 | ProteOn |
| C2 | GMA 5G8 | 7.92E−12 | 1.56E−12 | 5.94E−12 | 2.59E−12 | ProteOn |
| C2 | GMA 8025 | | | | | |
| C2 | GMA 8026 | | | | | |
| C2 | MBS17 | 6.59E−09 | 7.00E−10 | 8.52E−09 | 6.68E−10 | Biacore |

The present invention provides a method of reducing or preventing a FVIII-binding molecule from binding to a FVIII protein comprising contacting an anti-FVIII antibody or antigen-binding molecule thereof with the FVIII protein, wherein the anti-FVIII antibody or antigen-binding molecule thereof binds to a FVIII-binding site to which the FVIII-binding molecules binds. In one embodiment, the FVIII protein is present in a medium without the FVIII-binding molecule when it is contacted with the anti-FVIII antibody or antigen-binding molecule thereof selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17.

The anti-FVIII antibody or antigen binding molecule thereof useful for the method comprises:

(i) a variable heavy chain CDR-1 (VH-CDR1) sequence at least about 60%, 70%, 80%, 90%, 95%, or 100% identical to VH-CDR1 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17;

(ii) a variable heavy chain CDR-2 (VH-CDR2) sequence at least about 60%, 70%, 80%, 90%, 95%, or 100% identical to VH-CDR2 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17;

(iii) a variable heavy chain CDR-3 (VH-CDR3) sequence at least about 60%, 70%, 80%, 90%, 95%, or 100% identical to VH-CDR3 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17;

(iv) a variable light chain CDR-1 (VL-CDR1) sequence at least about 60%, 70%, 80%, 90%, 95%, or 100% identical to VL-CDR1 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17;

(v) a variable light chain CDR-2 (VL-CDR2) sequence at least about 60%, 70%, 80%, 90%, 95%, or 100% identical to VL-CDR2 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17; and,

(vi) a variable light chain CDR-3 (VL-CDR3) sequence at least about 60%, 70%, 80%, 90%, 95%, or 100% identical to VL-CDR3 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17, and

wherein the anti-FVIII antibody or antigen-binding molecule thereof specifically binds to a FVIII epitope, which is located in an A1 region, an A2 region, an A3 region, a C1 region, a C2 region, or any combinations thereof.

In some embodiments, the anti-FVIII antibody or antigen-binding molecule thereof comprises a VH region and a VL region,

wherein the VH region comprises an amino acid sequence at least 60%, at least 70%, at least 80%, at least 90%, at least 95%, or at least 100% identical to the VH region of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17; and

wherein the VL region comprises an amino acid sequence at least 60%, at least 70%, at least 80%, at least 90%, at least 95%, or at least 100% identical to the VL region of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA5G8, GMA5E8, GMA8025, GMA8026, or MBS17.

In one aspect, a method of the invention comprises contacting a FVIII protein with an anti-FVIII antibody or antigen-binding molecule thereof specifically binds to the A1 domain of FVIII. For example, non-limiting examples of the anti-FVIII antibody or antigen-binding molecule thereof specifically binds to the A1 domain of FVIII includes an antibody or antigen binding molecule thereof comprising the VH region of an antibody selected from GMA8002, GMA8004, or GMA8005 and the VL region of an antibody selected from GMA8002, GMA8004, or GMA8005.

In another aspect, the method comprises contacting a FVIII protein with an anti-FVIII antibody or antigen-binding molecule thereof specifically binds to the A2 domain of FVIII. Examples of the anti-FVIII antibody or antigen-binding molecule thereof includes, but are not limited to an anti-FVIII antibody or antigen-binding molecule thereof comprising the VH region of an antibody selected from GMA8023, GMA8024, GMA012, GMA8009, GMA8016, GMA8017, MBS11, MBS32, or MBS22 and the VL region of an antibody selected from GMA012, GMA8009, GMA8016, GMA8017, MBS11, MBS32, GMA8015, GMA8021, N772-10M, OBT-0037A, or MBS22.

In other aspects, the method comprises contacting a FVIII protein with an anti-FVIII antibody or antigen-binding molecule thereof specifically binds to the A3 domain of FVIII. In some examples, the anti-FVIII antibody or antigen-binding molecule thereof comprises the VH region of an antibody selected from GMA8001, GMA8010, MBS14, or GMA8019 and the VL region of an antibody selected from GMA8001, GMA8010, MBS14, or GMA8019.

In some aspects, the method comprises contacting a FVIII protein with an anti-FVIII antibody or antigen-binding molecule thereof specifically binds to the C1 domain of FVIII. Non-limiting examples of the anti-FVIII antibody or antigen-binding molecule thereof include an anti-FVIII antibody or antigen-binding molecule thereof comprising the VH region of an antibody selected from GMA8011, GMA8020, or MBS14, and the VL region of an antibody selected from GMA8011, GMA8020, or MBS14.

In other aspects, the method comprises contacting a FVIII protein with an anti-FVIII antibody or antigen-binding molecule thereof specifically binds to the C2 domain of FVIII. Examples of the anti-FVIII antibody or antigen-binding molecule thereof include, but are not limited to, an anti-FVIII antibody or antigen-binding molecule thereof comprising the VH region of an antibody selected from ESH4, ESH8, GMA8003, GMA8006, GMA8008, GMA8013, GMA8014, GMA8022, GMA8018, GMA8045, GMA5G8, GMA8025, GMA8026, or MBS17 and the VL region of an antibody selected from ESH4, ESH8, GMA8003, GMA8006, GMA8008, GMA8013, GMA8014, GMA8022, GMA8018, GMA8045, GMA5G8, GMA8025, GMA8026, or MBS17.

In certain aspects, the method comprises contacting a FVIII protein with an anti-FVIII antibody or antigen-binding molecule thereof specifically binds to a light chain. Non-limiting examples of the anti-FVIII antibody or antigen-binding molecule thereof include an anti-FVIII antibody or antigen-binding molecule thereof comprising the VH region of an antibody selected from GMA8013, GMA8018, GMA8020, GMA8011, GMA8010, GMA5E8, or GMA8019 and the VL region of an antibody selected from GMA8013, GMA8018, GMA8020, GMA8011, GMA8010, GMA5E8, or GMA8019.

In the absence of the FVIII-binding molecule, the anti-FVIII antibody or antigen-binding molecule thereof can freely bind to the FVIII protein, thereby blocking the binding site of the FVIII-binding molecule, e.g., a FVIII inhibitor, on FVIII. The method can further comprise adding the FVIII-binding molecule, e.g., a FVIII inhibitor, to the mixture of the FVIII protein and the anti-FVIII antibody or antigen-binding molecule thereof. The FVIII-binding molecule, e.g., a FVIII inhibitor, that binds to the same site as the anti-FVIII antibody or antigen-binding molecule is not capable of binding to the FVIII protein. In one embodiment, the method can further comprise measuring binding of the FVIII-binding molecule, e.g., a FVIII inhibitor. If the anti-FVIII antibody or antigen-binding molecule thereof is bound to the same binding site as the FVIII-binding molecule, e.g., a FVIII inhibitor, the measurement of the FVIII-binding molecule, e.g., a FVIII inhibitor, shows low or no binding to the FVIII protein.

In another embodiment, the method further comprises measuring binding of the anti-FVIII antibody or antigen-binding molecule thereof to the FVIII protein. In other embodiments, the method further comprises identifying the FVIII-binding molecule, e.g., a FVIII inhibitor, which does not bind to the FVIII protein in the presence of the anti-FVIII antibody or antigen-binding molecule thereof. In some embodiments, the method can be used to design a FVIII protein variant to identify a FVIII protein that does or does not bind to a FVIII-binding molecule, e.g., a FVIII inhibitor. Therefore, the method can further comprise identifying the FVIII protein, which does not bind to the FVIII-binding molecule, e.g., a FVIII inhibitor, in the presence of the anti-FVIII antibody or antigen-binding molecule thereof.

Also provided is a method of reducing or preventing a FVIII-binding molecule, e.g., a FVIII inhibitor, from binding to a FVIII protein comprising contacting an anti-FVIII antibody or antigen-binding molecule thereof with the FVIII protein, wherein the anti-FVIII antibody or antigen-binding molecule thereof binds to a FVIII-binding site to which the FVIII-binding molecules binds, wherein the FVIII protein is present in a medium comprising the FVIII-binding molecule, e.g., a FVIII inhibitor, and wherein the anti-FVIII antibody or antigen-binding molecule thereof competes the binding to the FVIII protein with the FVIII-binding molecule, e.g., a FVIII inhibitor. In one aspect, in the presence of the FVIII-binding molecule, the anti-FVIII antibody or antigen-binding molecule thereof competes with the FVIII-binding molecule, e.g., a FVIII inhibitor, thereby reducing the binding of the FVIII-binding molecule to the FVIII protein. Therefore, in some embodiments, the method further comprises measuring the binding of the FVIII-binding molecule, e.g., a FVIII inhibitor, to the FVIII protein. In other embodiments, the method further comprises measuring the binding of the anti-FVIII antibody or antigen-binding molecule thereof to the FVIII protein.

Measuring binding of the FVIII-binding molecule, e.g., FVIII inhibitor, can lead to identification of the FVIII-binding molecule. Thus, in some embodiments, a method further comprises identifying the FVIII-binding molecule, e.g., FVIII inhibitor, that competes the binding to the FVIII protein with the anti-FVIII antibody or antigen-binding molecule thereof. In other embodiments, the method can be used in designing a FVIII protein variant.

In certain embodiments, the invention includes a method of identifying a subject who has developed a FVIII inhibitor which binds to a FVIII protein in plasma comprising contacting an anti-FVIII antibody or antigen-binding molecule thereof with the plasma of the subject, wherein the anti-FVIII antibody or antigen-binding molecule thereof binds to a FVIII-binding site to which the FVIII inhibitor binds. In some examples, the method can further comprise measuring the binding of the anti-FVIII antibody or antigen-binding molecule thereof to the FVIII protein in the presence of the FVIII inhibitor. In other examples, the method further comprises identifying the subject who has developed the FVIII inhibitor, which prevents or inhibits specific binding of one or more of the anti-FVIII antibody or antigen-binding molecule thereof.

In other embodiments, the invention includes a method of identifying a FVIII binding site of a FVIII inhibitor comprising contacting an anti-FVIII antibody or antigen-binding molecule thereof with a FVII protein in the presence of the FVIII inhibitor. In some examples, the method further comprises selecting the anti-FVIII antibody or antigen-binding molecule thereof that competes the binding to the FVIII protein with the FVIII inhibitor. In other examples, the method further comprises identifying the FVIII binding site of the anti-FVIII antibody or antigen-binding molecule thereof. In yet other embodiments, the invention provides a method of preventing or inhibiting a cellular update of a FVIII protein comprising contacting an anti-FVIII antibody or antigen-binding molecule thereof with the FVIII protein which specifically binds to a FVIII epitope.

### IV. Factor VII Protein

“Factor VIII protein” or “FVIII protein” as used herein, means functional Factor VIII protein in its normal role in coagulation, unless otherwise specified. Thus, the term FVIII includes variant proteins that are functional. In one embodiment, the FVIII protein is the human, porcine, canine, rat, or murine FVIII protein. A functional FVIII protein can be a fusion protein, such as, but not limited to, a fusion protein comprising a fully or partially B-domain deleted FVIII, at least a portion of an immunoglobulin constant region, e.g., an Fc domain, or both. Myriad functional FVIII variants have been constructed and can be used as recombinant FVIII proteins as described herein. See PCT Publication Nos. WO 2011/069164 A2, WO 2012/006623 A2, WO 2012/006635 A2, or WO 2012/006633 A2, all of which are incorporated herein by reference in their entireties.

A great many functional FVIII variants are known. In addition, hundreds of nonfunctional mutations in FVIII have been identified in hemophilia patients. See, e.g., Cutler et al., Hum. Mutat. 19:274-8 (2002), incorporated herein by reference in its entirety. In addition, comparisons between FVIII from humans and other species have identified conserved residues that are likely to be required for function. See, e.g., Cameron et al., Thromb. Haemost. 79:317-22 (1998) and U.S. Pat. No. 6,251,632, incorporated herein by reference in their entireties.

The human FVIII amino acid sequence was deduced from cDNA as shown in U.S. Pat. No. 4,965,199, which is incorporated herein by reference in its entirety. Native mature human FVIII derived from the cDNA sequence (i.e., without the secretory signal peptide but prior to other post-translational processing) is presented as SEQ ID NO: 1. Partially or fully B-domain deleted FVIII is functional and has been used in commercial FVIII therapeutics. See, e.g., EP506757B2, which is incorporated herein by reference in its entirety.

“Native mature FVIII” comprises functional domains, which may or may not be necessary for procoagulant activity. The sequence of native mature human FVIII is presented as SEQ ID NO: 1. A native FVIII protein has the following formula: A1-a1-A2-a2-B-a3-A3-C1-C2, where A1, A2, and A3 are the structurally-related “A domains,” B is the “B domain,” C1 and C2 are the structurally-related “C domains,” and a1, a2 and a3 are acidic spacer regions.

In certain aspects a recombinant FVIII protein is provided, where the protein comprises a first polypeptide, i.e., an amino acid chain, comprising Formula I: (A1)-a1-(A2)-a2-[B], and a second polypeptide, i.e., an amino acid chain, comprising Formula II: a3-(A3)-(C1). The first polypeptide and the second polypeptide can exist as a single amino acid chain, that is, fused through amide bonds, or can exist as a heterodimer. According to this aspect, A1 is an A1 domain of FVIII as described herein, A2 is an A2 domain of FVIII as described herein, [B] is an optional B domain of FVIII or a fragment thereof (i.e., the B domain may or may not be part of the protein, and may be only partially present), A3 is an A3 domain of FVIII as described herein, C1 is a C1 domain of FVIII as described herein, and a1, a2, and a3 are acidic spacer regions. In certain aspects the second polypeptide further comprises a (C2) situated C-terminal to the (C1), where C2 is a C2 domain of FVIII. While the various FVIII domains of a recombinant polypeptide of the invention share primary sequence similarity with the corresponding regions of native mature FVIII, e.g., native mature human FVIII, the regions need not be identical provided that the recombinant polypeptide has procoagulant activity.

A FVIII protein of the invention can comprise at least one heterologous moiety inserted into at least one permissive loop, or into the a3 region, or both, has procoagulant activity, and can be expressed in a host cell. A “heterologous moiety” can be a heterologous polypeptide or a non-polypeptide entity, such as polyethylene glycol (PEG) or both. Exemplary heterologous moieties are described below. In certain aspects a recombinant FVIII protein of the invention comprises at least one heterologous moiety inserted into FVIII. The terms “insert” or “insert into” as applied to FVIII refer to the covalent or non-covalent attachment of heterologous moiety to a FVIII polypeptide by integrating it within the FVIII polypeptide chain, attaching it to the side chain of a native amino acid or a heterologous natural or non-natural amino acid (e.g., a cysteine or another amino acid with a derivatizable side chain introduced in the FVIII sequence using molecular biology methods), or to a linker or other molecule covalently or non-covalently attached to the FVIII polypeptide. The term “insertion” when used in the context of a polypeptide sequence refers to the introduction of a heterologous sequence (e.g., a polypeptide or a derivatizable amino acid such as cysteine) between two contiguous amino acids in the amino acid sequence of a FVIII polypeptide, or the fusion, conjugation, or chemical attachment of a heterologous moiety to a FVIII polypeptide.

In certain aspects, a FVIII protein of the invention is chimeric. A “chimeric protein,” or “chimeric polypeptide” as used herein, means a protein or polypeptide that includes within it at least two stretches of amino acids from different sources, e.g., a FVIII protein comprising a heterologous polypeptide, e.g., a VWF fragment, e.g., a D′ domain and a D3 domain of FVIII. Chimeric proteins or chimeric polypeptides can include two, three, four, five, six, seven, or more amino acid chains from different sources, such as different genes, different cDNAs, or different species. Exemplary heterologous polypeptides for use in recombinant polypeptides of the invention include, but are not limited to polypeptides which increase FVIII half-life or stability, for example, an immunoglobulin Fc region and/or a VWF fragment.

### IV.A. Heterologous Moieties

The heterologous moiety or moieties of the FVIII protein disclosed herein can comprise, consist of, or consist essentially of prophylactic and/or therapeutic agents (e.g., clotting factors), molecules capable of improving a pharmacokinetic (PK) property (e.g., plasma half-life extending moieties), detectable moieties (e.g., fluorescent molecules or radionuclides), etc.

As used herein, the term “therapeutic agent” refers to any biological or chemical agent used in the treatment of a disease or disorder. Therapeutic agents include any suitable biologically active chemical compounds, biologically derived components such as cells, peptides, antibodies, and polynucleotides, and radiochemical therapeutic agents such as radioisotopes. In some embodiments, the chimeric molecule comprises a clotting factor.

In some embodiments, a heterologous moiety can modify a physicochemical property of a FVIII protein lacking such heterologous moiety, for example, it can increase the hydrodynamic radius of a chimeric molecule. In other embodiments, the incorporation of a heterologous moiety into a FVIII protein can improve one or more pharmacokinetic properties without significantly affecting its biological activity or function.

In some embodiments, the heterologous moiety is a polypeptide comprising, consisting essentially of, or consisting of at least about 10, 100, 200, 300, 400, 500, 600, 700, 800, 900, 1000, 1100, 1200, 1300, 1400, 1500, 1600, 1700, 1800, 1900, 2000, 2500, 3000, or 4000 amino acids. In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In other embodiments, a heterologous moiety increases stability of the chimeric molecule of the invention or a fragment thereof. As used herein, the term “stability” refers to an art-recognized measure of the maintenance of one or more physical properties of the chimeric molecule in response to an environmental condition (e.g., an elevated or lowered temperature). In certain embodiments, the physical property can be the maintenance of the covalent structure of the chimeric molecule (e.g., the absence of proteolytic cleavage, unwanted oxidation or deamidation). In other embodiments, the physical property can also be the presence of the chimeric molecule in a properly folded state (e.g., the absence of soluble or insoluble aggregates or precipitates). In one embodiment, the stability of the chimeric molecule is measured by assaying a biophysical property of the chimeric molecule, for example thermal stability, pH unfolding profile, stable removal of glycosylation, solubility, biochemical function (e.g., ability to bind to a protein, receptor or ligand), etc., and/or combinations thereof. In another embodiment, biochemical function is demonstrated by the binding affinity of the interaction. In one embodiment, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In some embodiments, the FVIII protein comprises at last one heterologous moiety that is a “half-life extending moiety.” As used herein, the term “half-life extending moiety” refers to a heterologous moiety which increases the in vivo half-life of a protein, for example, a chimeric molecule. The term “half-life” refers to a biological half-life of a particular protein or polypeptide (e.g., a clotting factor or a chimeric molecule disclosed herein) in vivo. Half-life can be represented by the time required for half the quantity administered to a subject to be cleared from the circulation and/or other tissues in the animal. When a clearance curve of a given polypeptide or chimeric molecule of the invention is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, procoagulant compounds of the invention are monophasic, and thus do not have an alpha phase, but just the single beta phase. In certain embodiments, the term half-life as used herein refers to the half-life of the procoagulant compound in the β-phase. The typical β phase half-life of a human antibody in humans is 21 days. In vivo half-life of a chimeric molecule can be determined by any method known to those of skill in the art. In certain embodiments, the half-life extending moiety can comprise an attachment site for a non-polypeptide moiety (e.g., PEG).

Half-life extending moieties, as discussed below in detail, can comprise, for example, (i) low complexity peptides, (ii) albumin, (iii) albumin binding polypeptide or fatty acid, (iv) Fc, (v) transferrin, (vi) PAS, (vii) the C-terminal peptide (CTP) of the β subunit of human chorionic gonadotropin, (viii) polyethylene glycol (PEG), (ix) hydroxyethyl starch (HES), (x) albumin-binding small molecules, (xi) vWF, (xii) a clearance receptor or fragment thereof which blocks binding of the chimeric molecule to a clearance receptor, or (xiii) any combinations thereof. In some embodiments, the half-life extending moiety comprises an Fc region. In other embodiments, the half-life extending moiety comprises two Fc regions fused by a linker. Exemplary heterologous moieties also include, e.g., FcRn binding moieties (e.g., complete Fc regions or portions thereof which bind to FcRn), single chain Fc regions (scFc regions, e.g., as described in U.S. Publ. No. 2008-0260738, and Intl. Publ. Nos. WO 2008-012543 and WO 2008-1439545), or processable scFc regions. In some embodiments, a heterologous moiety can include an attachment site for a non-polypeptide moiety such as polyethylene glycol (PEG), hydroxyethyl starch (HES), polysialic acid, or any derivatives, variants, or combinations of these moieties.

In certain embodiments, a chimeric molecule of the invention comprises at least one half-like extending moiety which increases the in vivo half-life of the chimeric molecule with respect to the in vivo half-life of the corresponding chimeric molecule lacking such heterologous moiety. In vivo half-life of a chimeric molecule can be determined by any method known to those of skill in the art, e.g., activity assays (chromogenic assay or one stage clotting aPTT assay), ELISA, etc.

### EXAMPLES

### General Materials and Methods

In general, the practice of the present invention employs, unless otherwise indicated, conventional techniques of chemistry, biophysics, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., CS.H.L. Press, Pub. (1999); and Current Protocols in Molecular Biology, eds. Ausubel et al., John Wiley & Sons (1992).

### Example 1

### Pairwise Epitope Overlap Analysis—OCTET®

ForteBio's OCTET® was used to analyze the epitope overlap among the anti-FVIII antibodies. ForteBio's OCTET® utilizes BioLayer Interferometry (BLI) technology to monitor the interaction of proteins and other biomolecules to their binders directly in real time. The binding interaction is continuously detected by measuring the change in thickness of the protein layer on the sensor tip. The detector monitors the interference pattern created by attaching a layer of molecules to the tip of an optic fiber. Any change in the number of molecules bound results in a shift in the pattern. Monitoring the interference pattern vs. time allows sensitive detection on molecular binding.

Over thirty anti-FVIII mouse monoclonal antibodies, e.g., GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, or MBS17, were characterized. VIIISELECT® (a camelid nanobody (i.e., a single-chain antibody raised in llamas) was also characterized.

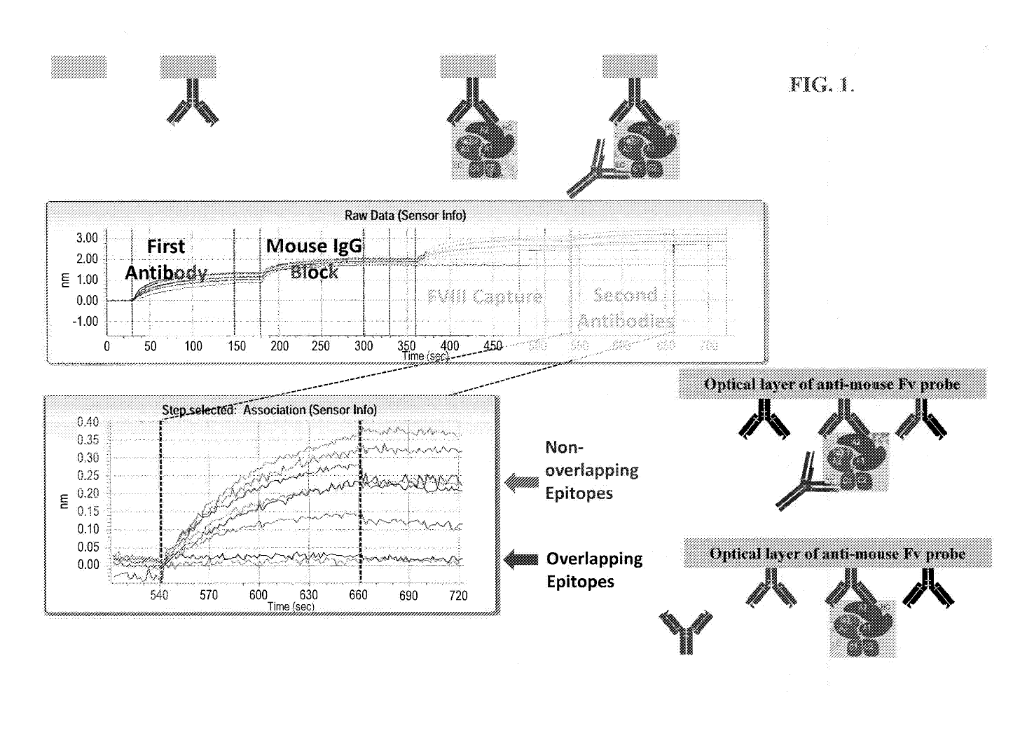

Anti-mouse Fv biosensor probe is used to bind the first antibody which then captures FVIII. Subsequently, the FVIII-bound probe is exposed to a second antibody. As FIG. 1 shows, an increase in the spectral interference signal upon exposure of the probe to the second antibody indicates non-competition between the two antibodies (upper schematic), whereas a lack of change in the signal indicates competition (lower schematic).

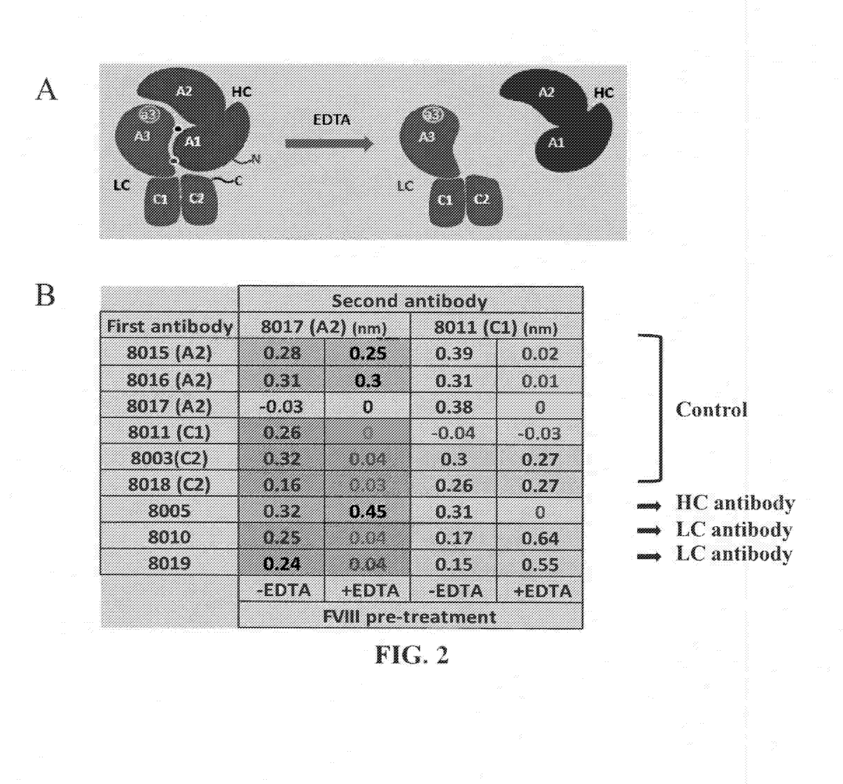

In order to determine the FVIII binding chain (heavy chain v. light chain), BDD rFVIII was incubated with EDTA in order to dissociate FVIII into separate heavy and light chains. Dissociated BDD rFVIII was then used in the assay as shown in FIG. 1. FIG. 2 shows an increase in the spectral interference signal (nm).

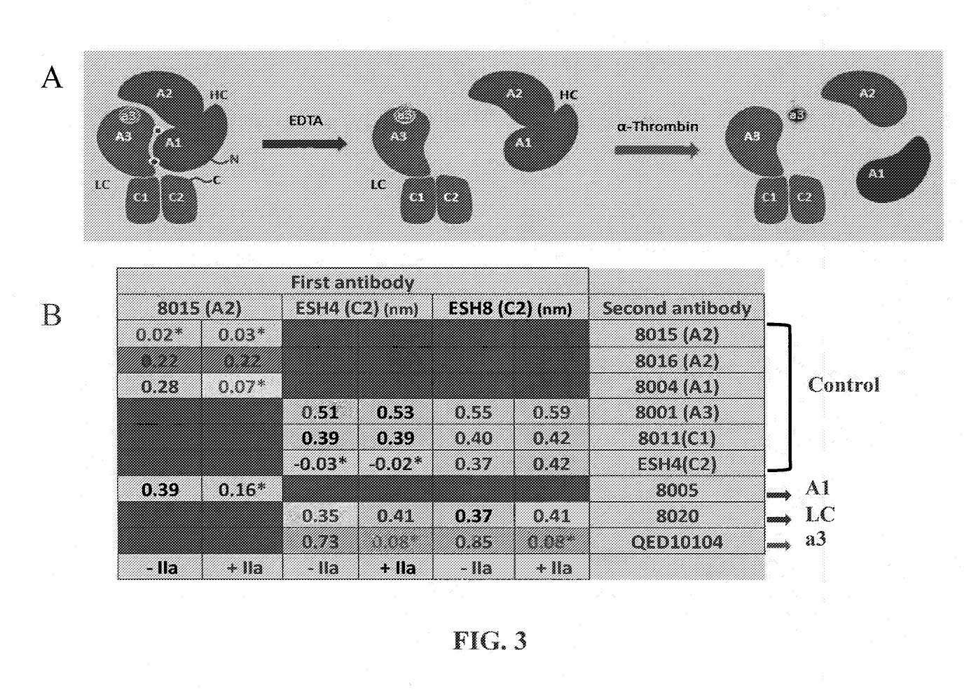

To determine the specific FVIII domain that each anti-FVIII antibody binds to, EDTA-dissociated BDD rFVIII was further digested with α-thrombin (IIa) to enable discrimination between the A domains in the heavy chain, as well as between the a3 acidic peptide and the remainder of the light chain. See FIG. 3A. Dissociated and digested BDD rFVIII was then used in the assay as shown in FIG. 1. As shown in FIG. 3B, increase in the spectral interference signal (nm) were shown. Mark (*) next to the numbers indicates no additional binding, i.e., different chain/domain, while the other numbers without (*) shows additional binding, or same chain/domain. Chain/domain specificity determined by these methods are shown at the right of FIG. 3B.

The anti-FVIII mouse monoclonal antibodies and VIIISELECT® that were characterized by Octet are summarized in a graph with domain and chain specificity indicated as shown in FIG. 5. The solid lines between MBS22 and 8016, between MBS14 and VIIISELECT®, between VIIISELECT® and 8011, between ESH4 and MBS17, and between VIIISELECT® and 8020 indicates partially overlapping epitopes. The other solid lines indicate overlapping epitopes. The dash lines indicate no overlapping epitopes. The halo rings indicate the FVIII antibodies that release FVII in buffers that maintain FVIII activity. These antibodies may be used for immunoaffinity purification.

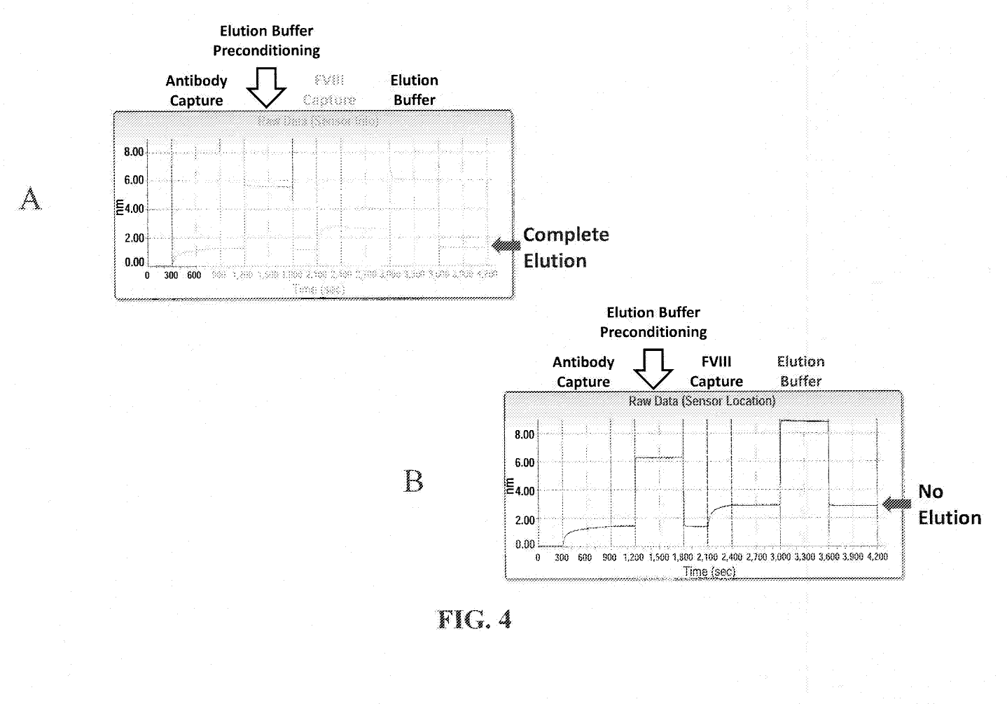

Several antibodies that were mapped to a specific domain, e.g., GMA8005 (A1), GMA8010 (A3), GMA8019 (A3), GMA8020 (C1), GMA8013 (C2), and GMA8018 (C2). Seven antibodies that are mapped to the FVIII C domains compete with VWF for binding to FVIII. Thirteen antibodies release FVIII in a buffer that maintains FVIII activity, indicating those antibodies have the potential to be used for affinity purification of FVIII (FIG. 4 and FIG. 5).

### Example 2

### Immunoaffinity Purification

In order to test its application for immunoaffinity purification, a test antibody was bound to an anti-mouse Fv biosensor probe. The probe was then exposed to an elution buffer for preconditioning. FVIII was captured by the antibody bound to the probe and then tested the release by the elution buffer (50 mM Histidine, 0.9 M Arginine HCl, 50 mM CaCl₂, 45% propylene glycol, 0.05% Tween20, pH 7.2). As shown in FIGS. 4A and 4B, upon exposure of the probe to an elution buffer, complete elution (left) shows a decrease in the spectral interference signal back to the level before FVIII binding while no elution (right) shows a lack of change in the signal.

### Example 3

### Affinity Measurement

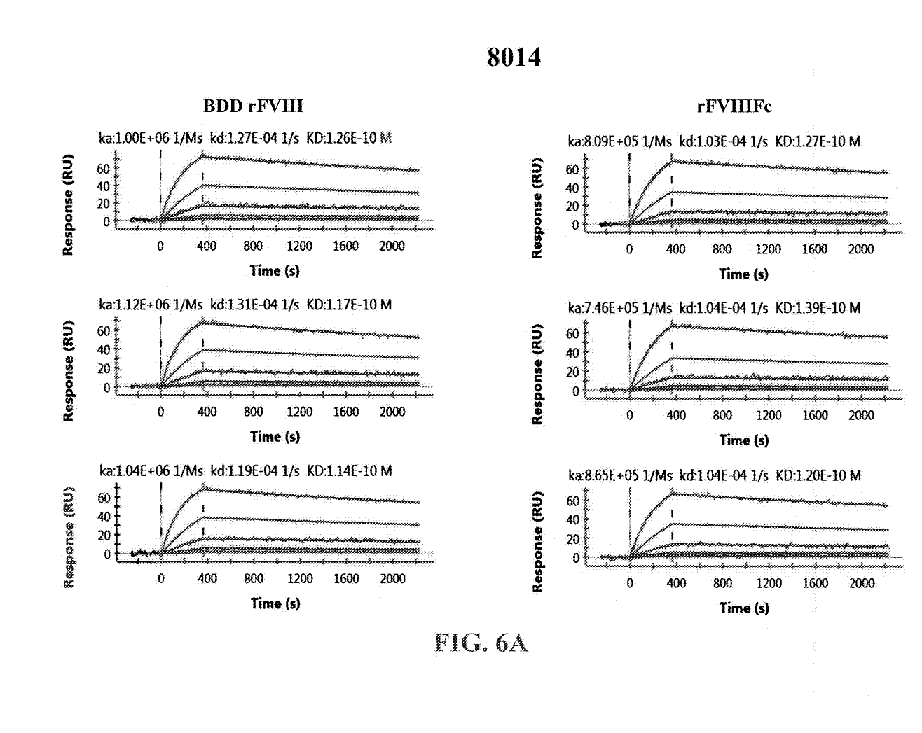

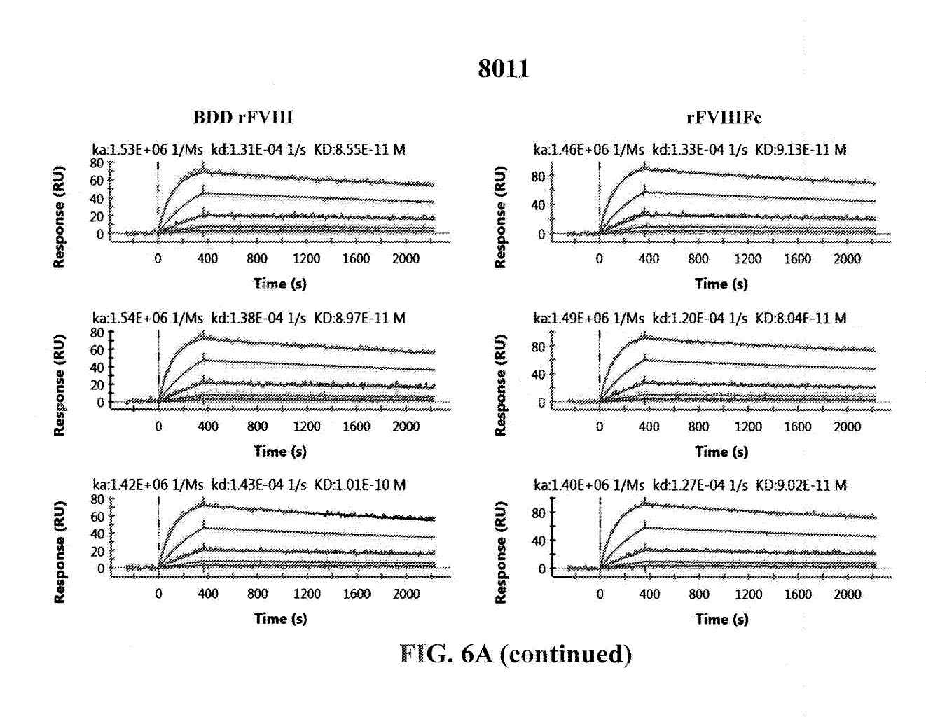

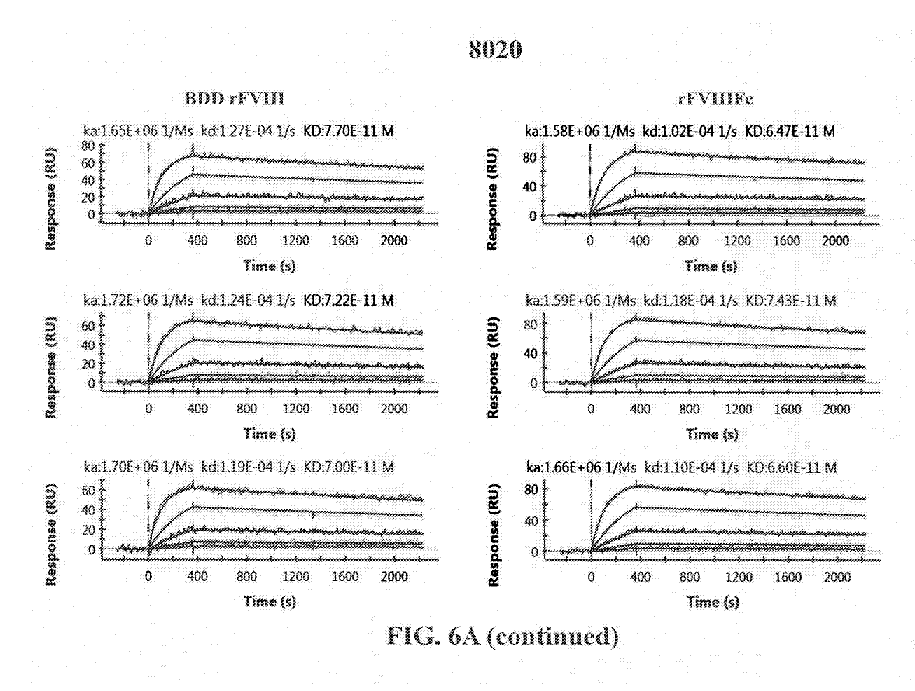

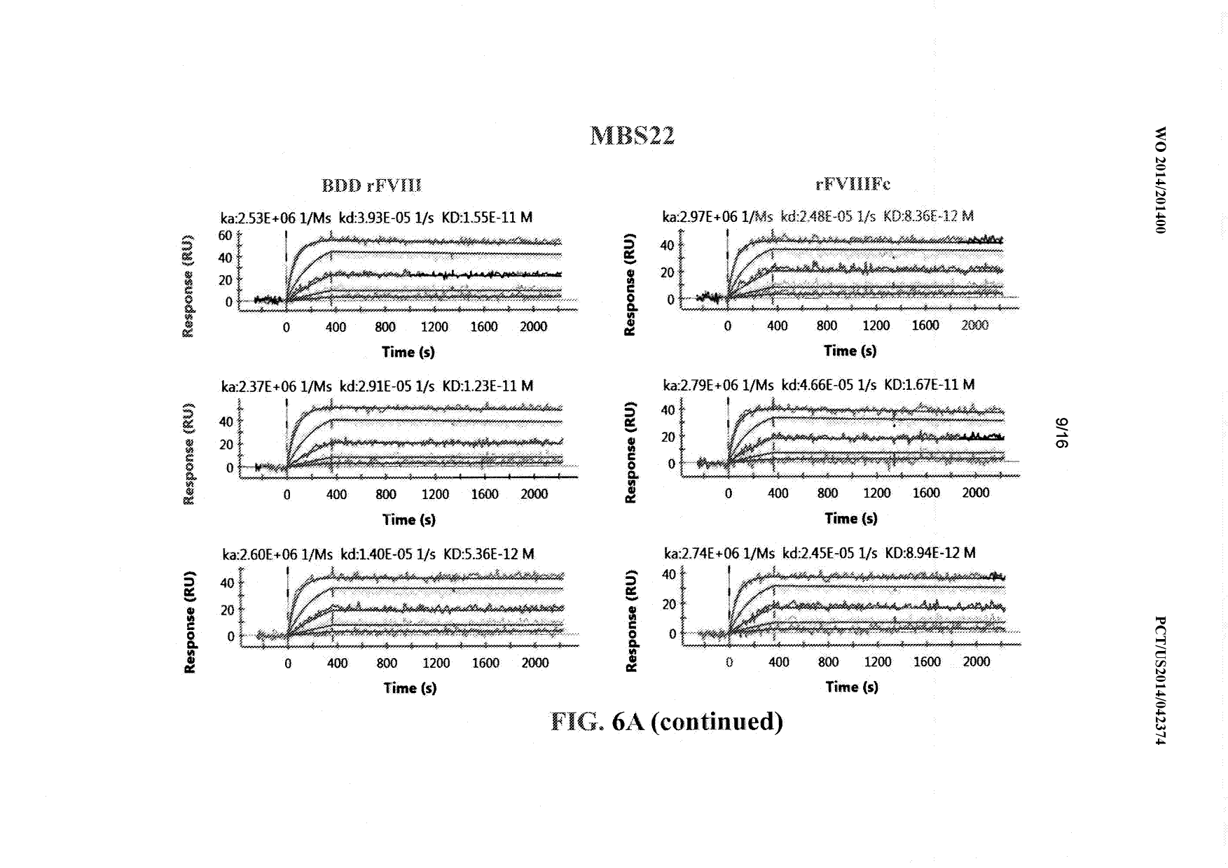

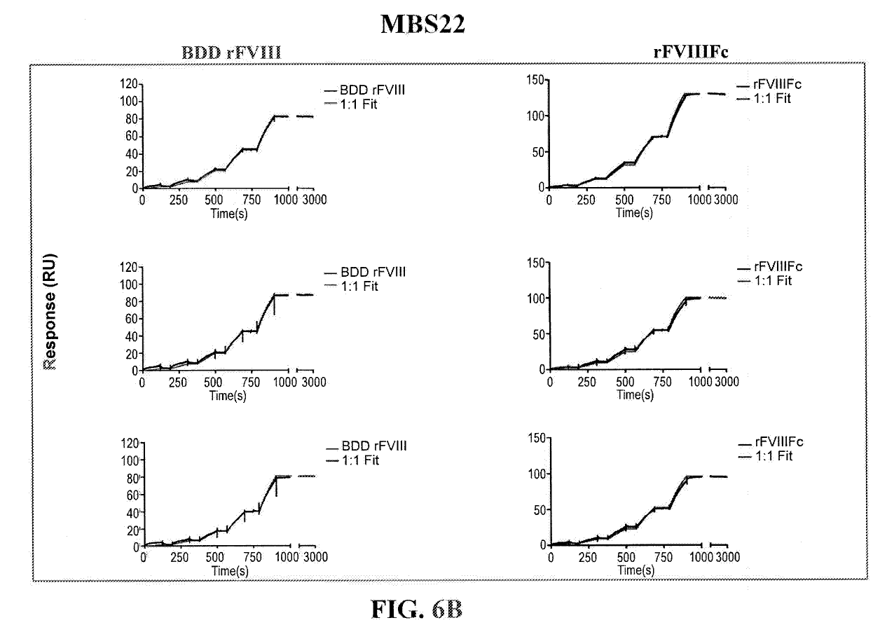

A total of 25 mouse monoclonal antibodies, representing specificities for all five structural domains of FVIII, were evaluated. FIG. 6A is representative triplicate experiments of the affinities of the GMA8014, GMA8020, GMA8011, and MBS22 antibodies for BDD rFVIII and rFVIIIFc on ProteOn. MBS22 showed very slow dissociation rate from rFVIIIFc or BDD rFVIII. To confirm the results of MBS22, as shown in FIG. 6B, the affinity was evaluated on Biacore by direct immobilization to the chip and by using long dissociation step (35 minutes). Results from both methods indicate similar affinity of MBS22 for rFVIIIFc and BDD rFVIII.

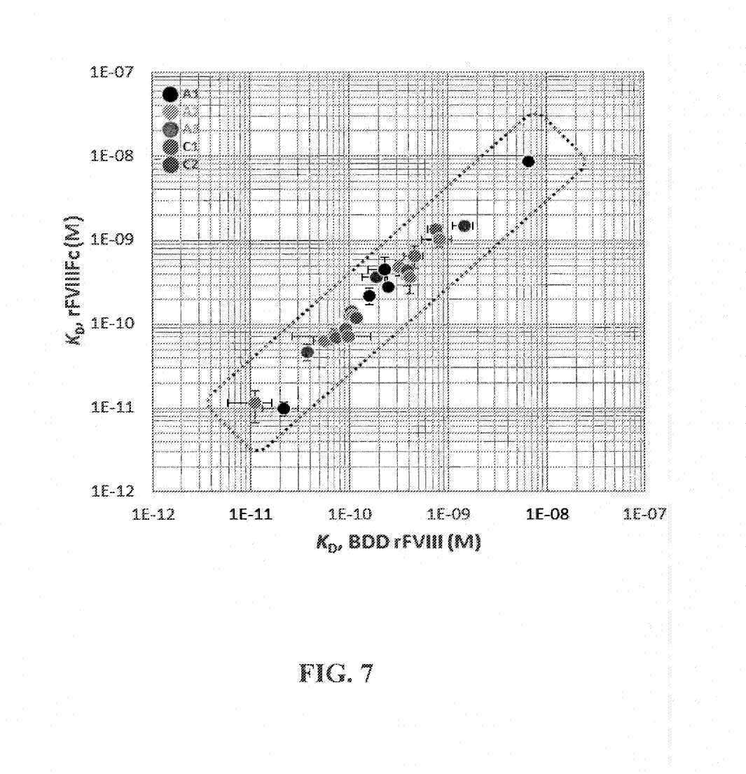

The affinities of the anti-FVIII antibodies were plotted as shown in FIG. 7. The y-axis shows rFVIIIFc affinity, and the x-axis shows BDD rFVIII affinity. The domain specificity of each antibody in the panel and the affinity is indicated in Table 1. The affinities of the antibodies for rFVIII and rFVIIIFc, expressed in terms of K_{D (M)}, spanned the picomolar to nanomolar range and were plotted against one another. The linear correlation indicates that the antibodies exhibit comparable affinities for rFVIII and rFVIIIFc.

### Example 4

### Negative strain Electron Microscopy (EM)

GMA-8015 (FIG. 8A) and ESH8 (FIG. 8B) anti-FVIII antibodies were selected for further analysis by negative stain EM. Fab fragments were generated for these two antibodies, and the structures of the Fab-rFVIII complexes were visualized. For both GMA-8015 Fab-FVIII and ESH8 Fab-FVIII, 5000 particles were grouped into 25 class averages as shown in the top panels. Representative images of the Fab-bound FVIII structures are shown in the lower panels. GMA-8015 binds to the A2 domain of FVIII, while ESH8 binds to the C2 domain.

For rFVIIIFc, negative stain EM images were grouped into 100 different classes as shown in FIG. 9A. The resulting class averages are shown in the top left panel of FIG. 9A, with a representative image of a single class shown in the lower left panel of FIG. 9A. This image represents one of the several possible orientations of the Fc relative to FVIII in rFVIIIFc. The class averages, comprising a total of 15,990 particles, were further divided into six groups representing different conformations rFVIIIFc. 3D maps were calculated from these groups and docked the crystal structures of rFVIII (3CDZ) and Fc (1HZH) into the resulting maps as shown in in the top left panel of FIG. 9B. The lower right panel of FIG. 9B illustrates one of the possible orientations of Fc relative to the FVIII component of rFVIIIFc in greater detail and from different views.

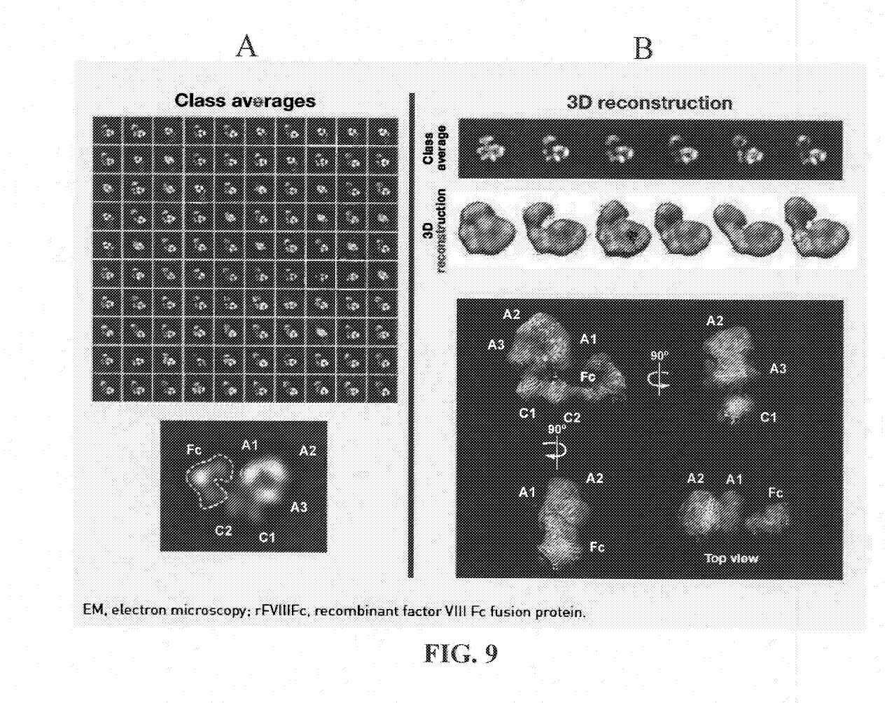

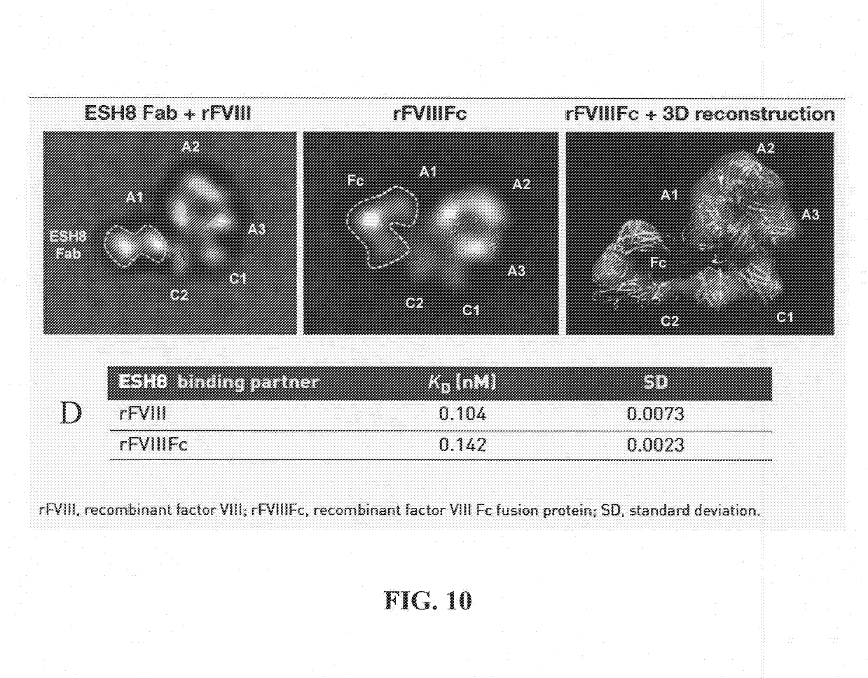

FIG. 10 shows representative negative stain EM images of ESH8 Fab-bound rFVIII (FIG. 10A), rFVIIIFc (FIG. 10B), and one of the 3D reconstructions of rFVIIIFc (FIG. 10C) in the same orientation. The ESH8 Fab binds to the C2 domain of rFVIII in close proximity to the Fc fusion site in rFVIIIFc. FIG. 10D shows that both rFVIII and rFVIIIFc bind to ESH8 with similar affinities, suggesting that the Fc does not interfere with the ESH8 interaction and can adopt multiple positions relative to the FVIII component of rFVIIIFc.

### Example 5

### Epitope Overlap Analysis—SPR

The PROTEON™ XPR36 protein interaction array was used to analyze the epitope overlap among the anti-FVIII antibodies. The ProteOn™ XPR36 protein interaction array system is a SPR optical biosensor that provides benefits of parallel processing. As other SPR technologies, The PROTEON™ XPR36 detects the refractive index change (mass change) within a thin layer on the surface of the sensor chip. This system generates a 6×6 interaction array for the simultaneous epitope overlap analysis of anti-FVIII antibodies.

Over thirty anti-FVIII mouse monoclonal antibodies, e.g., GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, MBS17, GMA8021, 8045, 5E8 or 5G8 were characterized.

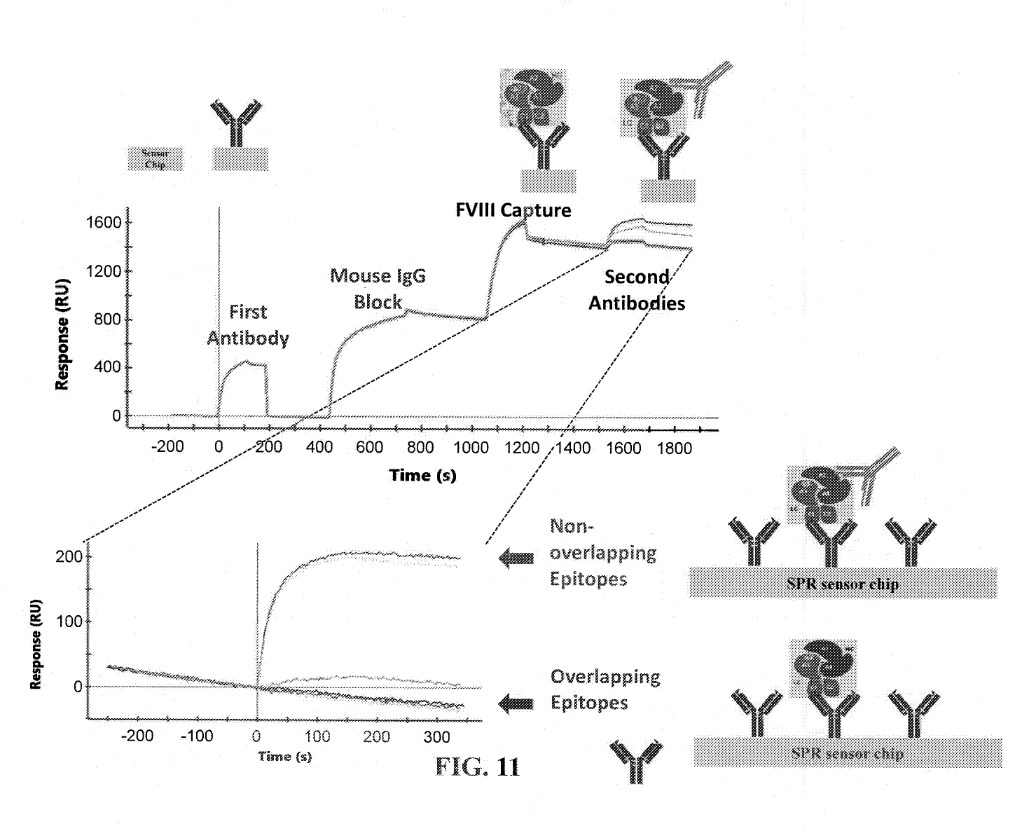

As FIG. 11 shows, GLC sensor chip was used for the epitope overlap analysis. First, goat anti mouse antibody was immobilized to the horizontal channels by amine coupling. First antibodies (capture antibodies) then flew through the chip vertically to be captured by goat anti-mouse antibody. The following steps were done horizontally in the order described. Mouse IgG was used to block goat anti-mouse antibody. FVIII was captured by first antibodies. Subsequently, the second antibodies flew through the FVIII-bound sensor chip. As FIG. 11 shows, an increase in the response signal upon exposure of the chip to the second antibody indicates non-competition between the two antibodies (upper schematic), whereas a lack of change in the signal indicates competition (lower schematic). FIG. 12 shows the epitope overlap analysis of the method.

The present invention has been described above with the aid of functional building blocks illustrating the implementation of specified functions and relationships thereof. The boundaries of these functional building blocks have been arbitrarily defined herein for the convenience of the description. Alternative boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents. Other embodiments of the invention will be apparent to those skilled in the art from consideration of the specification and practice of the invention disclosed herein.

All patents and publications cited herein are incorporated by reference herein in their entirety.

SEQUENCE LISTING:
SEQ ID NO: 1
>sp|P00451|20-2351 FA8_HUMAN Coagulation factor VIII, mature sequence (w/o
signal peptide)
ATRRYYLGAVELSWDYMQSDLGELPVDARFPPRVPKSFPFNTSVVYKKTLFVEFTDHLFNIAKPRPPWMGLLGPTIQA
EVYDTVVITLKNMASHPVSLHAVGVSYWKASEGAEYDDQTSQREKEDDKVFPGGSHTYVWQVLKENGPMASDPLCLTY
SYLSHVDLVKDLNSGLIGALLVCREGSLAKEKTQTLHKFILLFAVFDEGKSWHSETKNSLMQDRDAASARAWPKMHTV
NGYVNRSLPGLIGCHRKSVYWHVIGMGTTPEVHSIFLEGHTFLVRNHRQASLEISPITFLTAQTLLMDLGQFLLFCHI
SSHQHDGMEAYVKVDSCPEEPQLRMKNNEEAEDYDDDLTDSEMDVVRFDDDNSPSFIQIRSVAKKHPKTWVHYIAAEE
EDWDYAPLVLAPDDRSYKSQYLNNGPQRIGRKYKKVRFMAYTDETFKTREAIQHESGILGPLLYGEVGDTLLIIFKNQ
ASRPYNIYPHGITDVRPLYSRRLPKGVKHLKDFPILPGEIFKYKWTVTVEDGPTKSDPRCLTRYYSSFVNMERDLASG
LIGPLLICYKESVDQRGNQIMSDKRNVILFSVFDENRSWYLTENIQRFLPNPAGVQLEDPEFQASNIMHSINGYVFDS
LQLSVCLHEVAYWYILSIGAQTDFLSVFFSGYTFKHKMVYEDTLTLFPFSGETVFMSMENPGLWILGCHNSDFRNRGM
TALLKVSSCDKNTGDYYEDSYEDISAYLLSKNNAIEPRSFSQNSRHPSTRQKQFNATTIPENDIEKTDPWFAHRTPMP
KIQNVSSSDLLMLLRQSPTPHGLSLSDLQEAKYETFSDDPSPGAIDSNNSLSEMTHFRPQLHHSGDMVFTPESGLQLR
LNEKLGTTAATELKKLDFKVSSTSNNLISTIPSDNLAAGTDNTSSLGPPSMPVHYDSQLDTTLFGKKSSPLTESGGPL
SLSEENNDSKLLESGLMNSQESSWGKNVSSTESGRLFKGKRAHGPALLTKDNALFKVSISLLKTNKTSNNSATNRKTH
IDGPSLLIENSPSVWQNILESDTEFKKVTPLIHDRMLMDKNATALRLNHMSNKTTSSKNMEMVQQKKEGPIPPDAQNP
DMSFFKMLFLPESARWIQRTHGKNSLNSGQGPSPKQLVSLGPEKSVEGQNFLSEKNKVVVGKGEFTKDVGLKEMVFPS
SRNLFLTNLDNLHENNTHNQEKKIQEEIEKKETLIQENVVLPQIHTVTGTKNFMKNLFLLSTRQNVEGSYDGAYAPVL
QDFRSLNDSTNRTKKHTAHFSKKGEEENLEGLGNQTKQIVEKYACTTRISPNTSQQNFVTQRSKRALKQFRLPLEETE
LEKRIIVDDTSTQWSKNMKHLTPSTLTQIDYNEKEKGAITQSPLSDCLTRSHSIPQANRSPLPIAKVSSFPSIRPIYL
TRVLFQDNSSHLPAASYRKKDSGVQESSHFLQGAKKNNLSLAILTLEMTGDQREVGSLGTSATNSVTYKKVENTVLPK
PDLPKTSGKVELLPKVHIYQKDLFRTETSNGSFGHLDLVEGSLLQGTEGAIKWNEANRPGKVPFLRVATESSAKTPSK
LLDPLAWDNHYGTQIPKEEWKSQEKSPEKTAFKKKDTILSLNACESNHAIAAINEGQNKPEIEVTWAKQGRTERLCSQ
NPPVLKRHQREITRTTLQSDQEEIDYDDTISVEMKKEDFDIYDEDENQSPRSFQKKTRHYFIAAVERLWDYGMSSSPH
VLRNRAQSGSVPQFKKVVFQEFTDGSFTQPLYRGELNEHLGLLGPYIRAEVEDNIMVTFRNQASRPYSFYSSLISYEE
DQRQGAEPRKNFVKPNETKTYFWKVQHHMAPTKDEFDCKAWAYFSDVDLEKDVHSGLIGPLLVCHTNTLNPAHGRQVT
VQEFALFFTIFDETKSWYFTENMERNCRAPCNIQMEDPTFKENYRFHAINGYIMDTLPGLVMAQDQRIRWYLLSMGSN
ENIHSIHFSGHVFTVRKKEEYKMALYNLYPGVFETVEMLPSKAGIWRVECLIGEHLHAGMSTLFLVYSNKCQTPLGMA
SGHIRDFQITASGQYGQWAPKLARLHYSGSINAWSTKEPFSWIKVDLLAPMIIHGIKTQGARQKFSSLYISQFIIMYS
LDGKKWQTYRGNSTGTLMVFFGNVDSSGIKHNIFNPPIIARYIRLHPTHYSIRSTLRMELMGCDLNSCSMPLGMESKA
ISDAQITASSYFTNMFATWSPSKARLHLQGRSNAWRPQVNNPKEWLQVDFQKTMKVTGVTTQGVKSLLTSMYVKEFLI
SSSOGHQWTLFFQNGKVKVFQGNOSFTPVVNSLDPPLLTRYLRIHPQSWVHQIALRMEVLGCEAQDLY
SEQ ID NO: 2
>Mature B-Deleted FVIII construct, protein sequence
ATRRYYLGAVELSWDYMQSDLGELPVDARFPPRVPKSFPFNTSVVYKKTLFVEFTDHLFNIAKPRPPWMGLLGPTIQA
EVYDTVVITLKNMASHPVSLHAVGVSYWKASEGAEYDDQTSQREKEDDKVFPGGSHTYVWQVLKENGPMASDPLCLTY
SYLSHVDLVKDLNSGLIGALLVCREGSLAKEKTQTLHKFILLFAVFDEGKSWHSETKNSLMQDRDAASARAWPKMHTV
NGYVNRSLPGLIGCHRKSVYWHVIGMGTTPEVHSIFLEGHTFLVRNHRQASLEISPITFLTAQTLLMDLGQFLLFCHI
SSHQHDGMEAYVKVDSCPEEPQLRMKNNEEAEDYDDDLTDSEMDVVRFDDDNSPSFIQIRSVAKKHPKTWVHYIAAEE
EDWDYAPLVLAPDDRSYKSQYLNNGPQRIGRKYKKVRFMAYTDETFKTREAIQHESGILGPLLYGEVGDTLLIIFKNQ
ASRPYNIYPHGITDVRPLYSRRLPKGVKHLKDFPILPGEIFKYKWTVTVEDGPTKSDPRCLTRYYSSFVNMERDLASG
LIGPLLICYKESVDQRGNQIMSDKRNVILFSVFDENRSWYLTENIQRFLPNPAGVQLEDPEFQASNIMHSINGYVFDS
LQLSVCLHEVAYWYILSIGAQTDFLSVFFSGYTFKHKMVYEDTLTLFPFSGETVFMSMENPGLWILGCHNSDFRNRGM
TALLKVSSCDKNTGDYYEDSYEDISAYLLSKNNAIEPRSFSQNPPVLKRHQREITRTTLQSDQEEIDYDDTISVEMKK
EDFDIYDEDENQSPRSFQKKTRHYFIAAVERLWDYGMSSSPHVLRNRAQSGSVPQFKKVVFQEFTDGSFTQPLYRGEL
NEHLGLLGPYIRAEVEDNIMVTFRNQASRPYSFYSSLISYEEDQRQGAEPRKNFVKPNETKTYFWKVQHHMAPTKDEF
DCKAWAYFSDVDLEKDVHSGLIGPLLVCHTNTLNPAHGRQVTVQEFALFFTIFDETKSWYFTENMERNCRAPCNIQME
DPTFKENYRFHAINGYIMDTLPGLVMAQDQRIRWYLLSMGSNENTHSIHFSGHVFTVRKKEEYKMALYNLYPGVRETV
EMLPSKAGIWRVECLIGEHLHAGMSTLFLVYSNKCQTPLGMASGHIRDFQITASGQYGQWAPKLARLHYSGSINAWST
KEPFSWIKVDLLAPMIIHGIKTQGARQKFSSLYISQFIIMYSLDGKKWQTYRGNSTGTLMVFFGNVDSSGIKHNIFNP
PIIARYIRLHPTHYSIRSTLRMELMGCDLNSCSMPLGMESKAISDAQITASSYFTNMFATWSPSKARLHLQGRSNAWR
PQVNNPKEWLQVDFQKTMKVTGVTTQGVKSLLTSMYVKEFLISSSQDGHQWTLFFQNGKVKVFQGNQDSFTPVVNSLD
PPLLTRYLRIHPQSWVHQIALRMEVLGCEAQDLY
SEQ ID NO: 3
>Mature B-Deleted FVIII construct, DNA sequence
GCCACCAGAAGATACTACCTGGGTGCAGTGGAACTGTCATGGGACTATATGCAAAGTGATCTCGGTGAGCTGCCTGTG
GACGCAAGATTTCCTCCTAGAGTGCCAAAATCTTTTCCATTCAACACCTCAGTCGTGTACAAAAAGACTCTGTTTGTA
GAATTCACGGATCACCTTTTCAACATCGCTAAGCCAAGGCCACCCTGGATGGGTCTGCTAGGTCCTACCATCCAGGCT
GAGGTTTATGATACAGTGGTCATTACACTTAAGAACATGGCTTCCCATCCTGTCAGTCTTCATGCTGTTGGTGTATCC
TACTGGAAAGCTTCTGAGGGAGCTGAATATGATGATCAGACCAGTCAAAGGGAGAAAGAAGATGATAAAGTCTTCCCT
GGTGGAAGCCATACATATGTCTGGCAGGTCCTGAAAGAGAATGGTCCAATGGCCTCTGACCCACTGTGCCTTACCTAC
TCATATCTTTCTCATGTGGACCTGGTAAAAGACTTGAATTCAGGCCTCATTGGAGCCCTACTAGTATGTAGAGAAGGG
AGTCTGGCCAAGGAAAAGACACAGACCTTGCACAAATTTATACTACTTTTTGCTGTATTTGATGAAGGGAAAAGTTGG
CACTCAGAAACAAAGAACTCCTTGATGCAGGATAGGGATGCTGCATCTGCTCGGGCCTGGCCTAAAATGCACACAGTC
AATGGTTATGTAAACAGGTCTCTGCCAGGTCTGATTGGATGCCACAGGAAATCAGTCTATTGGCATGTGATTGGAATG
GGCACCACTCCTGAAGTGCACTCAATATTCCTCGAAGGTCACACATTTCTTGTGAGGAACCATCGCCAGGCTAGCTTG
GAAATCTCGCCAATAACTTTCCTTACTGCTCAAACACTCTTGATGGACCTTGGACAGTTTCTACTGTTTTGTCATATC
TCTTCCCACCAACATGATGGCATGGAAGCTTATGTCAAAGTAGACAGCTGTCCAGAGGAACCCCAACTACGAATGAAA
AATAATGAAGAAGCGGAAGACTATGATGATGATCTTACTGATTCTGAAATGGATGTGGTCAGGTTTGATGATGACAAC
TCTCCTTCCTTTATCCAAATTCGCTCAGTTGCCAAGAAGCATCCTAAAACTTGGGTACATTACATTGCTGCTGAAGAG
GAGGACTGGGACTATGCTCCCTTAGTCCTCGCCCCCGATGACAGAAGTTATAAAAGTCAATATTTGAACAATGGCCCT
CAGCGGATTGGTAGGAAGTACAAAAAAGTCCGATTTATGGCATACACAGATGAAACCTTTAAGACTCGTGAAGCTATT
CAGCATGAATCAGGAATCTTGGGACCTTTACTTTATGGGGAAGTTGGAGACACACTGTTGATTATATTTAAGAATCAA
GCAAGCAGACCATATAACATCTACCCTCACGGAATCACTGATGTCCGTCCTTTGTATTCAAGGAGATTACCAAAAGGT
GTAAAACATTTGAAGGATTTTCCAATTCTGCCAGGAGAAATATTCAAATATAAATGGACAGTGACTGTAGAAGATGGG
CCAACTAAATCAGATCCTCGGTGCCTGACCCGCTATTACTCTAGTTTCGTTAATATGGAGAGAGATCTAGCTTCAGGA
CTCATTGGCCCTCTCCTCATCTGCTACAAAGAATCTGTAGATCAAAGAGGAAACCAGATAATGTCAGACAAGAGGAAT
GTCATCCTGTTTTCTGTATTTGATGAGAACCGAAGCTGGTACCTCACAGAGAATATACAACGCTTTCTCCCCAATCCA
GCTGGAGTGCAGCTTGAGGATCCAGAGTTCCAAGCCTCCAACATCATGCACAGCATCAATGGCTATGTTTTTGATAGT
TTGCAGTTGTCAGTTTGTTTGCATGAGGTGGCATACTGGTACATTCTAAGCATTGGAGCACAGACTGACTTCCTTTCT
GTCTTCTTCTCTGGATATACCTTCAAACACAAAATGGTCTATGAAGACACACTCACCCTATTCCCATTCTCAGGAGAA
ACTGTCTTCATGTCGATGGAAAACCCAGGTCTATGGATTCTGGGGTGCCACAACTCAGACTTTCGGAACAGAGGCATG
ACCGCCTTACTGAAGGTTTCTAGTTGTGACAAGAACACTGGTGATTATTACGAGGACAGTTATGAAGATATTTCAGCA
TACTTGCTGAGTAAAAACAATGCCATTGAACCAAGAAGCTTCTCTCAAAACCCACCAGTCTTGAAACGCCATCAACGG
GAAATAACTCGTACTACTCTTCAGTCAGATCAAGAGGAAATCGATTATGATGATACCATATCAGTTGAAATGAAGAAG
GAAGATTTTGACATTTATGATGAGGATGAAAATCAGAGCCCCCGCAGCTTTCAAAAGAAAACACGACACTATTTTATT
GCTGCAGTGGAGAGGCTCTGGGATTATGGGATGAGTAGCTCCCCACATGTTCTAAGAAACAGGGCTCAGAGTGGCAGT
GTCCCTCAGTTCAAGAAAGTTGTTTTCCAGGAATTTACTGATGGCTCCTTTACTCAGCCCTTATACCGTGGAGAACTA
AATGAACATTTGGGACTCCTGGGGCCATATATAAGAGCAGAAGTTGAAGATAATATCATGGTAACTTTCAGAAATCAG
GCCTCTCGTCCCTATTCCTTCTATTCTAGCCTTATTTCTTATGAGGAAGATCAGAGGCAAGGAGCAGAACCTAGAAAA
AACTTTGTCAAGCCTAATGAAACCAAAACTTACTTTTGGAAAGTGCAACATCATATGGCACCCACTAAAGATGAGTTT
GACTGCAAAGCCTGGGCTTATTTCTCTGATGTTGACCTGGAAAAAGATGTGCACTCAGGCCTGATTGGACCCCTTCTG
GTCTGCCACACTAACACACTGAACCCTGCTCATGGGAGACAAGTGACAGTACAGGAATTTGCTCTGTTTTTCACCATC
TTTGATGAGACCAAAAGCTGGTACTTCACTGAAAATATGGAAAGAAACTGCAGGGCTCCCTGCAATATCCAGATGGAA
GATCCCACTTTTAAAGAGAATTATCGCTTCCATGCAATCAATGGCTACATAATGGATACACTACCTGGCTTAGTAATG
GCTCAGGATCAAAGGATTCGATGGTATCTGCTCAGCATGGGCAGCAATGAAAACATCCATTCTATTCATTTCAGTGGA
CATGTGTTCACTGTACGAAAAAAAGAGGAGTATAAAATGGCACTGTACAATCTCTATCCAGGTGTTTTTGAGACAGTG
GAAATGTTACCATCCAAAGCTGGAATTTGGCGGGTGGAATGCCTTATTGGCGAGCATCTACATGCTGGGATGAGCACA
CTTTTTCTGGTGTACAGCAATAAGTGTCAGACTCCCCTGGGAATGGCTTCTGGACACATTAGAGATTTTCAGATTACA
GCTTCAGGACAATATGGACAGTGGGCCCCAAAGCTGGCCAGACTTCATTATTCCGGATCAATCAATGCCTGGAGCACC
AAGGAGCCCTTTTCTTGGATCAAGGTGGATCTGTTGGCACCAATGATTATTCACGGCATCAAGACCCAGGGTGCCCGT
CAGAAGTTCTCCAGCCTCTACATCTCTCAGTTTATCATCATGTATAGTCTTGATGGGAAGAAGTGGCAGACTTATCGA
GGAAATTCCACTGGAACCTTAATGGTCTTCTTTGGCAATGTGGATTCATCTGGGATAAAACACAATATTTTTAACCCT
CCAATTATTGCTCGATACATCCGTTTGCACCCAACTCATTATAGCATTCGCAGCACTCTTCGCATGGAGTTGATGGGC
TGTGATTTAAATAGTTGCAGCATGCCATTGGGAATGGAGAGTAAAGCAATATCAGATGCACAGATTACTGCTTCATCC
TACTTTACCAATATGTTTGCCACCTGGTCTCCTTCAAAAGCTCGACTTCACCTCCAAGGGAGGAGTAATGCCTGGAGA
CCTCAGGTGAATAATCCAAAAGAGTGGCTGCAAGTGGACTTCCAGAAGACAATGAAAGTCACAGGAGTAACTACTCAG
GGAGTAAAATCTCTGCTTACCAGCATGTATGTGAAGGAGTTCCTCATCTCCAGCAGTCAAGATGGCCATCAGTGGACT
CTCTTTTTTCAGAATGGCAAAGTAAAGGTTTTTCAGGGAAATCAAGACTCCTTCACACCTGTGGTGAACTCTCTAGAC
CCACCGTTACTGACTCGCTACCTTCGAATTCACCCCCAGAGTTGGGTGCACCAGATTGCCCTGAGGATGGAGGTTCTG
GGCTGCGAGGCACAGGACCTCTAC

What is claimed is:

1. An anti-FVIII antibody or antigen-binding molecule thereof which specifically binds to a Factor VIII (“FVIII”) epitope, wherein the antibody or antigen-binding molecule thereof exhibits one or more of the following characteristics:
(a) the anti-FVIII antibody or antigen-binding molecule thereof specifically binds to the same FVIII epitope as an antibody selected from MBS11, MBS32, MBS22, MBS14, GMA8023, GMA8024, GMA8045, GMA8025, GMA5G8, GMA8026, or MBS17;
(b) the anti-FVIII antibody or antigen-binding molecule thereof competitively inhibits FVII binding by an antibody selected from MBS11, MBS32, MBS22, MBS14, GMA8023, GMA8024, GMA8045, GMA8025, GMA5G8, GMA8026, or MBS17; or
(c) the anti-FVIII antibody or antigen-binding molecule thereof comprises at least one, at least two, at least three, at least four, or at least five complementarity determining regions (CDR) or variants thereof of an antibody selected from MBS11, MBS32, MBS22, MBS14, GMA8023, GMA8024, GMA8045, GMA8025, GMA5G8, GMA8026, or MBS17;
wherein the FVIII epitope is located in an A2 domain, an A3 domain, a C1 domain, a C2 domain, or any combinations thereof; and
wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

2. The anti-FVIII antibody or antigen-binding molecule thereof of claim 1, which comprises six CDRs or variants thereof of an antibody selected from MBS11, MBS32, MBS22, MBS14, GMA8023, GMA8024, GMA8045, GMA8025, GMA5G8, GMA8026, or MBS17.

3. An anti-FVIII antibody or antigen-binding molecule thereof which specifically binds to a FVIII epitope comprising:
(i) a variable heavy chain CDR-1 (VH-CDR1) sequence at least about 60%, 70%, 80%, 90%, 95%, or 100% identical to a VH-CDR1 of an antibody selected from MBS11, MBS32, GMA8023, GMA8024, or MBS22;
(ii) a variable heavy chain CDR-2 (VH-CDR2) sequence at least about 60%, 70%, 80%, 90%, 95%, or 100% identical to a VH-CDR2 of an antibody selected from MBS11, MBS32, GMA8023, GMA8024, or MBS22;
(iii) a variable heavy chain CDR-3 (VH-CDR3) sequence at least about 60%, 70%, 80%, 90%, 95%, or 100% identical to a VH-CDR3 of an antibody selected from MBS11, MBS32, GMA8023, GMA8024, or MBS22;
(iv) a variable light chain CDR-1 (VL-CDR1) sequence at least about 60%, 70%, 80%, 90%, 95%, or 100% identical to a VL-CDR1 of an antibody selected from MBS11, MBS32, GMA8023, GMA8024, or MBS22;
(v) a variable light chain CDR-2 (VL-CDR2) sequence at least about 60%, 70%, 80%, 90%, 95%, or 100% identical to a VL-CDR2 of an antibody selected from MBS11, MBS32, GMA8023, GMA8024, or MBS22; and,
(vi) a variable light chain CDR-3 (VL-CDR3) sequence at least about 60%, 70%, 80%, 90%, 95%, or 100% identical to a VL-CDR3 of an antibody selected from MBS11, MBS32, GMA8023, GMA8024, or MBS22,
wherein the FVIII epitope is located in an A2 domain, and
wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

4. The antibody or antigen-binding molecule thereof of claim 3, comprising
(i) the VH-CDR1 of an antibody selected from MBS11, MBS32, GMA8023, GMA8024, or MBS22;
(ii) the VH-CDR2 of an antibody selected from MBS11, MBS32, GMA8023, GMA8024, or MBS22;
(iii) the VH-CDR3 of an antibody selected from MBS11, MBS32, GMA8023, GMA8024, or MBS22;
(iv) the VL-CDR of an antibody selected from MBS11, MBS32, GMA8023, GMA8024, or MBS22;
(v) the VL-CDR2 of an antibody selected from MBS11, MBS32, GMA8023, GMA8024, or MBS22; and,
(vi) the VL-CDR3 of an antibody selected from MBS11, MBS32, GMA8023, GMA8024, or MBS22.

5. An anti-FVIII antibody or antigen-binding molecule thereof which specifically binds to an FVIII epitope, comprising a VH region, which comprises an amino acid sequence at least about 80%, 85%, 90%, 95%, or 100% identical to a VH of an antibody selected from MBS11, MBS32, GMA8023, GMA8024, or MBS22 and a VL region, which comprises an amino acid sequence at least about 80%, 85%, 90%, 95%, or 100% identical to a VL of an antibody selected from MBS1, MBS32, GMA8023, GMA8024, or MBS22,
wherein the FVIII epitope is located in an A2 domain and
wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

6. The antibody or antigen-binding molecule thereof of any one of claims 3 to 5, comprising the VH of MBS11 and the VL of MBS11 (MBS11 antibody).

7. The antibody or antigen-binding molecule thereof of any one of claims 3 to 5, comprising the VH of MBS32 and the VL of MBS32 (MBS32 antibody).

8. The antibody or antigen-binding molecule thereof of any one of claims 3 to 5, comprising the VH of MBS22 and the VL of MBS22 (MBS22 antibody).

9. An anti-FVIII antibody or antigen-binding molecule thereof which specifically binds to a FVIII epitope comprising
(i) a variable heavy chain CDR-1 (VH-CDR1) sequence at least about 60%, 70%, 80%, 90%, 95%, or 100% identical to a VH-CDR1 of MBS14;
(ii) a variable heavy chain CDR-2 (VH-CDR2) sequence at least about 60%, 70%, 80%, 90%, 95%, or 100% identical to a VH-CDR2 of MBS14;
(iii) a variable heavy chain CDR-3 (VH-CDR3) sequence at least about 60%, 70%, 80%, 90%, 95%, or 100% identical to a VH-CDR3 of MBS14;
(iv) a variable light chain CDR-1 (VL-CDR1) sequence at least about 60%, 70%, 80%, 90%, 95%, or 100% identical to a VL-CDR1 of MBS14;
(v) a variable light chain CDR-2 (VL-CDR2) sequence at least about 60%, 70%, 80%, 90%, 95%, or 100% identical to a VL-CDR2 of MBS14; and,
(vi) a variable light chain CDR-3 (VL-CDR3) sequence at least about 60%, 70%, 80%, 90%, 95%, or 100% identical to a VL-CDR3 of MBS14,
wherein the FVIII epitope is located in a C1 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

10. The antibody or antigen-binding molecule thereof of claim 9, comprising
(i) the VH-CDR1 of MBS14;
(ii) the VH-CDR2 of MBS14;
(iii) the VH-CDR3 of MBS14;
(iv) the VL-CDR1 of MBS14;
(v) the VL-CDR2 of MBS14; and,
(vi) the VL-CDR3 of MBS14.

11. An anti-FVIII antibody or antigen-binding molecule thereof which specifically binds to a FVIII epitope, comprising a VH region, which comprises an amino acid sequence at least about 80%, 85%, 90%, 95%, or 100% identical to the VH of MBS14 and a VL region, which comprises an amino acid sequence at least about 80%, 85%, 90%, 95%, or 100% identical to the VL of MBS14, wherein the FVIII epitope is a C1 domain and wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

12. The antibody or antigen-binding molecule thereof of any one of claims 9 to 11, comprising the VH of MBS14 and the VL of MBS14 (MBS14 antibody).

13. An anti-FVIII antibody or antigen-binding molecule thereof which specifically binds to a FVIII epitope comprising
(i) a variable heavy chain CDR-1 (VH-CDR1) sequence at least about 60%, 70%, 80%, 90%, 95%, or 100% identical to a VH-CDR1 of MBS17, GMA8045, GMA8025, GMA5G8, or GMA8026;
(ii) a variable heavy chain CDR-2 (VH-CDR2) sequence at least about 60%, 70%, 80%, 90%, 95%, or 100% identical to a VH-CDR2 of MBS17, GMA8045, GMA8025, GMA5G8, or GMA8026;
(iii) a variable heavy chain CDR-3 (VH-CDR3) sequence at least about 60%, 70%, 80%, 90%, 95%, or 100% identical to a VH-CDR3 of MBS17, GMA8045, GMA8025, GMA5G8, or GMA8026;
(iv) a variable light chain CDR-1 (VL-CDR1) sequence at least about 60%, 70%, 80%, 90%, 95%, or 100% identical to a VL-CDR1 of MBS17, GMA8045, GMA8025, GMA5G8, or GMA8026;
(v) a variable light chain CDR-2 (VL-CDR2) sequence at least about 60%, 70%, 80%, 90%, 95%, or 100% identical to a VL-CDR2 of MBS7, GMA8045, GMA8025, GMA5G8, or GMA8026; and,
(vi) a variable light chain CDR-3 (VL-CDR3) sequence at least about 60%, 70%, 80%, 90%, 95%, or 100% identical to a VL-CDR3 of MBS17, GMA8045, GMA8025, GMA5G8, or GMA8026,
wherein the FVIII epitope is located in a C2 domain and
wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

14. The antibody or antigen-binding molecule thereof of claim 13, comprising
(i) the VH-CDR1 of MBS17, GMA8045, GMA8025, GMA5G8, or GMA8026;
(ii) the VH-CDR2 of MBS17, GMA8045, GMA8025, GMA5G8, or GMA8026;
(iii) the VH-CDR3 of MBS17, GMA8045, GMA8025, GMA5G8, or GMA8026;
(iv) the VL-CDR1 of MBS17, GMA8045, GMA8025, GMA5G8, or GMA8026;
(v) the VL-CDR2 of MBS17, GMA8045, GMA8025, GMA5G8, or GMA8026; and,
(vi) the VL-CDR3 of MBS17, GMA8045, GMA8025, GMA5G8, or GMA8026.

15. An anti-FVIII antibody or antigen-binding molecule thereof which specifically binds to a FVIII epitope, comprising a VH region, which comprises an amino acid sequence at least about 80%, 85%, 90%, 95%, or 100% identical to the VH of MBS17, GMA8045, GMA8025, GMA5G8, or GMA8026 and a VL region, which comprises an amino acid sequence at least about 80%, 85%, 90%, 95%, or 100% identical to the VL of MBS17, GMA8045, GMA8025, GMA5G8, or GMA8026,
wherein the FVIII epitope is located in a C2 domain and
wherein the anti-FVIII antibody or antigen binding molecule thereof is not GMA8002, GMA8005, GMA8004, GMA012, GMA8009, GMA8015, GMA8016, GMA8017, GMA8001, GMA8010, GMA8019, GMA8011, GMA8020, ESH4, ESH8, GMA8003, GMA8006, GMA8008, GMA8013, GMA8014, GMA8018, GMA8022, VIIISELECT®, N772-10M, or OBT-0037A.

16. The antibody or antigen-binding molecule thereof of claim 15, comprising the VH of MBS17, GMA8045, GMA8025, GMA5G8, or GMA8026 and the VL of MBS17, GMA8045, GMA8025, GMA5G8, or GMA8026.

17. The antibody or antigen-binding molecule thereof of any one of claims 1 to 16, which is a monoclonal antibody, a chimeric antibody, or a humanized antibody.

18. The antibody or antigen-binding molecule thereof of any one of claims 1 to 16, which is:
(a) a single chain Fv (“scFv”);
(b) a diabody;
(c) a minibody;
(d) a polypeptide chain of an antibody;
(e) F(ab′)2; or,
(f) F(ab).

19. A nucleic acid molecule or a set of nucleic acid molecules encoding the anti-FVIII antibody or antigen-binding molecule thereof of any one of claims 1 to 18 or a complement thereof.

20. A vector or a set of vectors comprising the nucleic acid molecule or the set of the nucleic acid molecules of claim 19 or a complement thereof.

21. A host cell comprising the vector of claim 20 or the set of vectors of claim 20.

22. A composition comprising the anti-FVIII antibody or antigen-binding molecule thereof of any one of claims 1 to 18, the nucleic acid molecule or the set of nucleic acid molecules of claim 19, the vector or the set of vectors of claim 20 and a carrier.

23. A kit comprising the anti-FVIII antibody or antigen-binding molecule thereof of any one of claims 1 to 18, the nucleic acid molecule or the set of nucleic acid molecules of claim 19, the vector or the set of vectors of claim 20 and a packaging material.

24. A method for producing an anti-FVIII antibody or antigen-binding molecule thereof comprising culturing the host cell of claim 21 and recovering the anti-FVIII antibody or antigen-binding molecule thereof from the culture medium.

25. A method of purifying a FVIII protein, comprising contacting an anti-FVIII antibody or antigen-binding molecule thereof, which specifically binds to a FVIII epitope, with the FVIII protein and eluting the FVIII protein in a buffer, wherein the anti-FVIII antibody or antigen-binding molecule thereof comprises:
(i) a variable heavy chain CDR-1 (VH-CDR1) sequence at least about 60%, 70%, 80%, 90%, 95%, or 100% identical to a VH-CDR1 of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006;
(ii) a variable heavy chain CDR-2 (VH-CDR2) sequence at least about 60%, 70%, 80%, 90%, 95%, or 100% identical to a VH-CDR2 of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006;
(iii) a variable heavy chain CDR-3 (VH-CDR3) sequence at least about 60%, 70%, 80%, 90%, 95%, or 100% identical to a VH-CDR3 of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006;
(iv) a variable light chain CDR-1 (VL-CDR1) sequence at least about 60%, 70%, 80%, 90%, 95%, or 100% identical to a VL-CDR1 of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006;
(v) a variable light chain CDR-2 (VL-CDR2) sequence at least about 60%, 70%, 80%, 90%, 95%, or 100% identical to a VL-CDR2 of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006; and,
(vi) a variable light chain CDR-3 (VL-CDR3) sequence at least about 60%, 70%, 80%, 90%, 95%, or 100% identical to a VL-CDR3 of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006,
wherein the FVIII epitope is located in an A1 domain, an A2 domain, an A3 domain, a C1 domain, a C2 domain, a light chain, or any combinations thereof, and
wherein the buffer comprises propylene glycol and arginine.

26. The method of claim 25, wherein the anti-FVIII antibody or antigen-binding molecule thereof comprises a VH region and a VL region,
wherein the VH region comprises an amino acid sequence at least 60%, at least 70%, at least 80%, at least 90%, at least 95%, or at least 100% identical to a VH region of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006 and
wherein the VL region comprises an amino acid sequence at least 60%, at least 70%, at least 80%, at least 90%, at least 95%, or at least 100% identical to a VL region of an antibody selected from GMA8002, GMA8021, GMA8016, MBS32, GMA012, N772-10M, OBT-0037A, GMA8001, MBS14, GMA8020, GMA8011, GMA8013, GMA5G8, GMA5E8, GMA8023, GMA8024, GMA8026, or GMA8006.

27. The method of claim 25 or 26, wherein the FVIII epitope is located in the A1 domain.

28. The method of claim 27, wherein the anti-FVIII antibody or antigen-binding molecule thereof comprises a VH region and a VL region, wherein the VH region comprises the VH of GMA8002 and the VL region comprises the VL of GMA8002.

29. The method of claim 25 or 26, wherein the FVIII epitope is located in the A2 domain.

30. The method of claim 29, wherein the anti-FVIII antibody or antigen-binding molecule thereof comprises a VH region and a VL region, wherein the VH region comprises the VH of an antibody selected from GMA8021, GMA8016, MBS32, N772-10M, GMA012, or OBT-0037A and the VL region comprises the VL of an antibody selected from GMA8021, GMA8016, MBS32, N772-10M, GMA012, GMA8023, GMA8024, or OBT-0037A.

31. The method of claim 25 or 26, wherein the FVIII epitope is located in the A3 domain.

32. The method of claim 31, wherein the anti-FVIII antibody or antigen-binding molecule thereof comprises a VH region and a VL region, wherein the VH region comprises the VH of GMA8001 or MBS14 and the VL region comprises the VL of GMA8001 or MBS14.

33. The method of claim 25 or 26, wherein the FVIII epitope is located in the C1 domain.

34. The method of claim 33, wherein the anti-FVIII antibody or antigen-binding molecule thereof comprises a VH region and a VL region, wherein the VH region comprises the VH of GMA8011 and the VL region comprises the VL of GMA8011.

35. The method of claim 25 or 26, wherein the FVIII epitope is located in the C2 domain.

36. The method of claim 35, wherein the anti-FVIII antibody or antigen-binding molecule thereof comprises a VH region and a VL region, wherein the VH region comprises the VH of GMA8006, GMA8026, or GMA5G8, and the VL region comprises the VL of GMA 8006, GMA8026, or GMA5G8.

37. The method of claim 25 or 26, wherein the FVIII epitope is located in a light chain of the FVIII protein.

38. The method of claim 37, wherein the anti-FVIII antibody or antigen-binding molecule thereof comprises a VH region and a VL region, wherein the VH region comprises the VH of GMA8020, GMA5E8, or GMA8013 and the VL region comprises the VL of GMA8020, GMA5E8, or GMA8013.

39. The method of any one of claims 25 to 38, wherein the anti-FVIII antibody or antigen-binding molecule thereof binds to the FVIII protein at a dissociation constant (K_{D}) lower than about 1 nM.

40. The method of any one of claims 25 to 38, wherein the anti-FVIII antibody or antigen-binding molecule thereof binds to the FVIII protein at a dissociation constant (K_{D}) lower than about 0.1 nM.

41. The method of any one of claims 25 to 38, wherein the anti-FVIII antibody or antigen-binding molecule thereof binds to the FVIII protein at a dissociation constant (K_{D}) lower than about 0.01 nM.

42. A method of reducing or preventing a FVIII protein from binding to von Willebrand Factor (“VWF”) comprising contacting an anti-FVIII antibody or antigen-binding molecule thereof with the FVIII protein, wherein the anti-FVIII antibody or antigen-binding molecule thereof binds to the FVII protein at a VWF binding site.

43. The method of claim 42, wherein the anti-FVIII antibody or antigen-binding molecule thereof binds to the FVIII protein in the absence of VWF.

44. The method of claim 43, further comprising adding VWF to the mixture of the FVIII protein and the anti-FVIII antibody or antigen-binding molecule thereof.

45. The method of any one of claims 42 to 44, wherein the anti-FVIII antibody or antigen-binding molecule thereof competes the binding to the FVIII protein with VWF in the presence of VWF.

46. The method of any one of claims 42 to 45, further comprising measuring the binding of the FVIII protein to the anti-FVIII antibody or antigen-binding molecule thereof.

47. A method of identifying a FVIII protein that does not bind to VWF comprising contacting an anti-FVIII antibody or antigen-binding molecule thereof with the FVIII protein, measuring the binding of the anti-FVIII antibody or antigen-binding molecule thereof with the FVIII protein, and isolating the FVIII protein that does not bind to the anti-FVIII antibody or antigen-binding molecule thereof.

48. The method of any one of claims 42 to 47, wherein the VWF comprises full-length mature VWF.

49. The method of any one of claims 42 to 47, wherein the VWF is a VWF fragment.

50. The method of any one of claims 42 to 47, wherein the VWF is endogenous VWF.

51. The method of any one of claims 42 to 50, wherein the FVIII protein has a half-life longer than the half-life of a protein consisting of full-length wild-type FVIII.

52. The method of any one of claims 42 to 50, further comprising administering the isolated FVIII protein to a subject in need thereof.

53. The method of claim 52, wherein the subject has hemophilia A.

54. The method of any one of claims 42 to 53, wherein the anti-FVIII antibody or antigen-binding molecule thereof comprises:
(i) a variable heavy chain CDR-1 (VH-CDR1) sequence at least about 60%, 70%, 80%, 90%, 95%, or 100% identical to VH-CDR1 of an antibody selected from GMA8018, MBS17, ESH4, GMA8013, GMA8008, GMA8011, GMA8045, or GMA8020;
(ii) a variable heavy chain CDR-2 (VH-CDR2) sequence at least about 60%, 70%, 80%, 90%, 95%, or 100% identical to VH-CDR2 of an antibody selected from GMA8018, MBS17, ESH4, GMA8013, GMA8008, GMA8011, GMA8045, or GMA8020;
(iii) a variable heavy chain CDR-3 (VH-CDR3) sequence at least about 60%, 70%, 80%, 90%, 95%, or 100% identical to VH-CDR3 of an antibody selected from GMA8018, MBS17, ESH4, GMA8013, GMA8008, GMA8011, GMA8045, or GMA8020;
(iv) a variable light chain CDR-1 (VL-CDR1) sequence at least about 60%, 70%, 80%, 90%/a, 95%, or 100% identical to VL-CDR1 of an antibody selected from GMA8018, MBS17, ESH4, GMA8013, GMA8008, GMA8011, GMA8045, or GMA8020;
(v) a variable light chain CDR-2 (VL-CDR2) sequence at least about 60%, 70%, 80%, 90%, 95%, or 100% identical to VL-CDR2 of an antibody selected from GMA8018, MBS17, ESH4, GMA8013, GMA8008, GMA8011, GMA8045, or GMA8020; and,
(vi) a variable light chain CDR-3 (VL-CDR3) sequence at least about 60%, 70%, 80%, 90%, 95%, or 100% identical to VL-CDR3 of an antibody selected from GMA8018, MBS17, ESH4, GMA8013, GMA8008, GMA8011, GMA8045, or GMA8020, and
wherein the anti-FVIII antibody or antigen-binding molecule thereof specifically binds to the VWF binding site on the FVIII protein.

55. The method of any one of claims 42 to 54, wherein the anti-FVIII antibody or antigen-binding molecule thereof comprises a VH region and a VL region,
wherein the VH region comprises an amino acid sequence at least 60%, at least 70%, at least 80%, at least 90%, at least 95%, or at least 100% identical to a VH region of an antibody selected from GMA8018, MBS17, ESH4, GMA8013, GMA8008, GMA8011, GMA8045, or GMA8020;
wherein the VL region comprises an amino acid sequence at least 60%, at least 70%, at least 80%/a, at least 90%, at least 95%, or at least 100% identical to a VL region of an antibody selected from GMA8018, MBS17, ESH4, GMA8013, GMA8008, GMA8011, GMA8045, or GMA8020; and
wherein the anti-FVIII antibody or antigen-binding molecule thereof specifically binds to the VWF binding site on the FVIII protein.

56. The method of claim 55, wherein the anti-FVIII antibody or antigen-binding molecule thereof comprises a VH region and a VL region, wherein the VH region comprises the VH of GMA8018 and the VL region comprises the VL of GMA8018.

57. The method of claim 55, wherein the anti-FVIII antibody or antigen-binding molecule thereof comprises a VH region and a VL region, wherein the VH region comprises the VH of MBS17 and the VL region comprises the VL of MBS17.

58. The method of claim 55, wherein the anti-FVIII antibody or antigen-binding molecule thereof comprises a VH region and a VL region, wherein the VH region comprises the VH of ESH4 and the VL region comprises the VL of ESH4.

59. The method of claim 55, wherein the anti-FVIII antibody or antigen-binding molecule thereof comprises a VH region and a VL region, wherein the VH region comprises the VH of GMA8013 and the VL region comprises the VL of GMA8013.

60. The method of claim 55, wherein the anti-FVIII antibody or antigen-binding molecule thereof comprises a VH region and a VL region, wherein the VH region comprises the VH of GMA8008 and the VL region comprises the VL of GMA8008.

61. The method of claim 55, wherein the anti-FVIII antibody or antigen-binding molecule thereof comprises a VH region and a VL region, wherein the VH region comprises the VH of GMA8011 and the VL region comprises the VL of GMA8011.

62. The method of claim 55, wherein the anti-FVIII antibody or antigen-binding molecule thereof comprises a VH region and a VL region, wherein the VH region comprises the VH of GMA8020 and the VL region comprises the VL of GMA8020.

63. A method of reducing or preventing a FVIII-binding molecule from binding to a FVIII protein comprising contacting an anti-FVIII antibody or antigen-binding molecule thereof with the FVIII protein, wherein the anti-FVIII antibody or antigen-binding molecule thereof binds to a FVIII-binding site to which the FVIII-binding molecules binds.

64. The method of claim 63, wherein the FVIII protein is present in a medium without the FVIII-binding molecule.

65. The method of claim 63 or 64, further comprising adding the FVIII-binding molecule to the mixture of the FVIII protein and the anti-FVIII antibody or antigen-binding molecule thereof.

66. The method of any one of claims 63 to 65, further comprising measuring binding of the FVIII-binding molecule or the anti-FVIII antibody or antigen-binding molecule thereof to the FVIII protein.

67. The method of any one of claims 63 to 66, further identifying the FVIII-binding molecule, which does not bind to the FVIII protein in the presence of the anti-FVIII antibody or antigen-binding molecule thereof.

68. The method of any one of claims 63 to 66, further identifying the FVIII protein, which does not bind to the FVIII-binding molecule in the presence of the anti-FVIII antibody or antigen-binding molecule thereof.

69. The method of claim 63, wherein the FVIII protein is present in a medium comprising the FVIII-binding molecule and wherein the anti-FVIII antibody or antigen-binding molecule thereof competes the binding to the FVIII protein with the FVIII-binding molecule.

70. The method of claim 63 or 69, further comprising measuring the binding of the FVIII-binding molecule or the anti-FVIII antibody or antigen-binding molecule thereof to the FVIII protein.

71. The method of any one of claims 63, 69, or 70, further comprising identifying the FVIII protein that competes the binding to the anti-FVIII antibody or antigen-binding molecule thereof with the FVIII-binding molecule.

72. The method of any one of claims 63, 69, 70, or 71, further comprising identifying the FVIII-binding molecule that competes the binding to the FVIII protein with the anti-FVIII antibody or antigen-binding molecule thereof.

73. A method of identifying a subject who has developed a FVIII inhibitor which binds to a FVIII protein in plasma comprising contacting an anti-FVIII antibody or antigen-binding molecule thereof with the plasma of the subject, wherein the anti-FVIII antibody or antigen-binding molecule thereof binds to a FVIII-binding site to which the FVIII inhibitor binds.

74. The method of claim 73, further comprising measuring the binding of the anti-FVIII antibody or antigen-binding molecule thereof to the FVIII protein in the presence of the FVIII inhibitor.

75. The method of claim 73 or 74, further comprising identifying the subject who has developed the FVIII inhibitor, which prevents or inhibits specific binding of one or more of the anti-FVIII antibody or antigen-binding molecule thereof.

76. A method of identifying a FVIII binding site of a FVIII inhibitor comprising contacting an anti-FVIII antibody or antigen-binding molecule thereof with a FVII protein in the presence of the FVIII inhibitor.

77. The method of claim 76, further comprising selecting the anti-FVIII antibody or antigen-binding molecule thereof that competes the binding to the FVIII protein with the FVIII inhibitor.

78. The method of claim 76 or 77, further comprising identifying the FVIII binding site of the anti-FVIII antibody or antigen-binding molecule thereof.

79. A method of preventing or inhibiting a cellular update of a FVIII protein comprising contacting an anti-FVIII antibody or antigen-binding molecule thereof with the FVIII protein which specifically binds to a FVIII epitope.

80. The method of any one of claims 63 to 79, wherein the anti-FVIII antibody or antigen-binding molecule thereof comprises:
(i) a variable heavy chain CDR-1 (VH-CDR1) sequence at least about 60%, 70%, 80%, 90%, 95%, or 100% identical to VH-CDR1 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;
(ii) a variable heavy chain CDR-2 (VH-CDR2) sequence at least about 60%, 70%, 80%, 90%, 95%, or 100% identical to VH-CDR2 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;
(iii) a variable heavy chain CDR-3 (VH-CDR3) sequence at least about 60%, 70%, 80%, 90%, 95%, or 100% identical to VH-CDR3 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;
(iv) a variable light chain CDR-1 (VL-CDR1) sequence at least about 60%, 70%, 80%, 90%, 95%, or 100% identical to VL-CDR1 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17;
(v) a variable light chain CDR-2 (VL-CDR2) sequence at least about 60%, 70%, 80%, 90%, 95%, or 100% identical to VL-CDR2 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17; and,
(vi) a variable light chain CDR-3 (VL-CDR3) sequence at least about 60%, 70%, 80%, 90%, 95%, or 100% identical to VL-CDR3 of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17, and
wherein the anti-FVIII antibody or antigen-binding molecule thereof specifically binds to a FVIII epitope, which is located in an A1 region, an A2 region, an A3 region, a C1 region, a C2 region, or any combinations thereof.

81. The method of any one of claims 63 to 80, wherein the anti-FVIII antibody or antigen-binding molecule thereof comprises a VH region and a VL region,
wherein the VH region comprises an amino acid sequence at least 60%, at least 70%, at least 80%, at least 90%, at least 95%, or at least 100% identical to the VH region of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17; and
wherein the VL region comprises an amino acid sequence at least 60%, at least 70%, at least 80%, at least 90%, at least 95%, or at least 100% identical to the VL region of an antibody selected from GMA8002, GMA8005, GMA8004, GMA012, GMA8009, GMA8015, GMA8016, GMA8017, MBS11, MBS32, MBS22, GMA8001, GMA8010, GMA8019, GMA8011, GMA8020, MBS14, ESH4, ESH8, GMA8003, GMA8006, GMA8008, GMA8013, GMA8014, GMA8018, GMA8022, GMA8023, GMA8024, GMA8045, GMA8025, GMA5G8, GMA5E8, GMA8026, or MBS17.

82. The method of any one of claims 63 to 81, wherein the anti-FVIII antibody or antigen-binding molecule thereof specifically binds to the A1 domain of FVIII.

83. The method of claim 82, wherein the anti-FVIII antibody or antigen-binding molecule thereof comprises the VH region of an antibody selected from GMA8002, GMA8004, or GMA8005 and the VL region of an antibody selected from GMA8002, GMA8004, or GMA8005.

84. The method of any one of claims 63 to 81, wherein the anti-FVIII antibody or antigen-binding molecule thereof specifically binds to the A2 domain of FVIII.

85. The method of claim 84, wherein the anti-FVIII antibody or antigen-binding molecule thereof comprises the VH region of an antibody selected from GMA012, GMA8009, GMA8016, GMA8017, MBS11, MBS32, GMA8024, GMA8023, or MBS22 and the VL region of an antibody selected from GMA012, GMA8009, GMA8016, GMA8017, MBS11, MBS32, GMA8015, GMA8021, N772-10M, OBT-0037A, GMA8024, GMA8023, or MBS22.

86. The method of any one of claims 63 to 81, wherein the anti-FVIII antibody or antigen-binding molecule thereof specifically binds to the A3 domain of FVIII.

87. The method of claim 86, wherein the anti-FVIII antibody or antigen-binding molecule thereof comprises the VH region of an antibody selected from GMA8001, GMA8010, MBS14, or GMA8019 and the VL region of an antibody selected from GMA8001, GMA8010, MBS14, or GMA8019.

88. The method of any one of claims 63 to 81, wherein the anti-FVIII antibody or antigen-binding molecule thereof specifically binds to a C1 domain of FVIII.

89. The method of claim 88, wherein the anti-FVIII antibody or antigen-binding molecule thereof comprises the VH region of an antibody selected from GMA8011, GMA8020, or MBS14, and the VL region of an antibody selected from GMA8011, GMA8020, or MBS14.

90. The method of any one of claims 63 to 81, wherein the anti-FVIII antibody or antigen-binding molecule thereof specifically binds to a C2 domain of FVIII.

91. The method of claim 90, wherein the anti-FVIII antibody or antigen-binding molecule thereof comprises the VH region of an antibody selected from ESH4, ESH8, GMA8003, GMA8006, GMA8008, GMA8013, GMA8014, GMA8022, GMA8018, GMA8045, GMA8025, GMA5G8, GMA8026, or MBS17 and the VL region of an antibody selected from ESH4, ESH8, GMA8003, GMA8006, GMA8008, GMA8013, GMA8014, GMA8022, GMA8018, GMA8045, GMA8025, GMA5G8, GMA8026, or MBS17.

92. The method of any one of claims 63 to 81, wherein the anti-FVIII antibody or antigen-binding molecule thereof specifically binds to a light chain.

93. The method of claim 92, wherein the anti-FVIII antibody or antigen-binding molecule thereof comprises the VH region of an antibody selected from GMA8013, GMA8018, GMA8020, GMA8010, GMA5E8, or GMA8019 and the VL region of an antibody selected from GMA8013, GMA8018, GMA8020, GMA8010, GMA5E8, or GMA8019.

94. The method of any one of claims 25 to 41, wherein the buffer is an elution buffer comprising at least about 30% (v/v), at least about 40% (v/v), at least about 45% (v/v), at least about 50% (v/v), at least about 55% (v/v), at least about 60% (v/v), at least about 65% (v/v), at least about 70% (v/v), or at least about 75% (v/v) propylene glycol.

95. The method of any one of claims 25 to 41, wherein the buffer is an elution buffer comprising at least about 0.5M, at least about 0.6M, at least about 0.7M, at least about 0.8M, at least about 0.9M, at least about 1.0M, at least about 1.1M, at least about 1.2M, or at least about 1.3M arginine.

96. The method of claim 94 or 95, wherein the buffer comprises about 30% (v/v) to about 80% (v/v) propylene glycol, about 40% (v/v) to about 70% (v/v) propylene glycol, about 40% (v/v) to about 60% (v/v) propylene glycol, or about 40% (v/v) to about 50% (v/v) propylene glycol.

97. The method of any one of claims 94 to 96, wherein the buffer comprises about 0.5M to about 2M of arginine.

98. The method of any one of claims 25 to 41 and 94 to 97, wherein the buffer further comprises histidine, CaCl₂, Tween-20, or any combinations thereof.

99. The method of claim 98, wherein the buffer comprises about 10 mM to about 100 mM of histidine, about 0.1M to about 1.5M of arginine, about 10 mM to about 100 mM of CaCl₂, about 30% (v/v/) to about 70% (v/v/) of propylene glycol, and about 0.01% to about 0.1% of Tween-20.

100. The method of claim 98, wherein the buffer comprises about 50 mM histidine, about 0.9M arginine, about 50 mM CaCl₂, about 45% (v/v) propylene glycol, and about 0.05% Tween-20 at pH 7.2.

101. A method of purifying a FVIII protein, comprising contacting an anti-FVIII antibody or antigen-binding molecule thereof, which specifically binds to a FVIII epitope, with the FVIII protein and eluting the FVIII protein in a high ionic buffer, wherein the anti-FVIII antibody or antigen-binding molecule thereof comprises:
(i) a variable heavy chain CDR-1 (VH-CDR1) sequence at least about 60%, 70%, 80%, 90%, 95%, or 100% identical to a VH-CDR1 of an antibody selected from ESH4, GMA8013, MBS17, GMA5G8, GMA5E8, GMA8025, or OBT0037A;
(ii) a variable heavy chain CDR-2 (VH-CDR2) sequence at least about 60%, 70%, 80%, 90%, 95%, or 100% identical to a VH-CDR2 of an antibody selected from ESH4, GMA8013, MBS17, GMA5G8, GMA5E8, GMA8025, or OBT0037A;
(iii) a variable heavy chain CDR-3 (VH-CDR3) sequence at least about 60%, 70%, 80%, 90%, 95%, or 100% identical to a VH-CDR3 of an antibody selected from ESH4, GMA8013, MBS17, GMA5G8, GMA5E8, GMA8025, or OBT0037A;
(iv) a variable light chain CDR-1 (VL-CDR1) sequence at least about 60%, 70%, 80%, 90%, 95%, or 100% identical to a VL-CDR1 of an antibody selected from ESH4, GMA8013, MBS17, GMA5G8, GMA5E8, GMA8025, or OBT0037A;
(v) a variable light chain CDR-2 (VL-CDR2) sequence at least about 60%, 70%, 80%, 90%, 95%, or 100% identical to a VL-CDR2 of an antibody selected from ESH4, GMA8013, MBS17, GMA5G8, GMA5E8, GMA8025, or OBT0037A; and,
(vi) a variable light chain CDR-3 (VL-CDR3) sequence at least about 60%, 70%, 80%, 90%, 95%, or 100% identical to a VL-CDR3 of an antibody selected from ESH4, GMA8013, MBS17, GMA5G8, GMA5E8, GMA8025, or OBT0037A,
wherein the FVIII epitope is located in a light chain, an A2 region, a C2 domain, or any combinations thereof.

102. The method of claim 101, wherein the anti-FVIII antibody or antigen-binding molecule thereof comprises a VH region and a VL region,
wherein the VH region comprises an amino acid sequence at least 60%, at least 70%, at least 80%, at least 90%, at least 95%, or at least 100% identical to a VH region of an antibody selected from ESH4, GMA8013, MBS17, GMA5G8, GMA5E8, GMA8025, or OBT0037A and
wherein the VL region comprises an amino acid sequence at least 60%, at least 70%, at least 80%, at least 90%, at least 95%, or at least 100% identical to a VL region of an antibody selected from ESH4, GMA8013, MBS17, GMA5G8, GMA5E8, GMA8025, or OBT0037A.

103. The method of claim 101 or 102, wherein the FVIII epitope is located in the C2 domain.

104. The method of claim 103, wherein the anti-FVIII antibody or antigen-binding molecule thereof comprises a VH region and a VL region, wherein the VH region comprises the VH of ESH4, GMA5G8, or GMA8025 and the VL region comprises the VL of ESH4, GMA5G8, or GMA8025.

105. The method of claim 103, wherein the anti-FVIII antibody or antigen-binding molecule thereof comprises a VH region and a VL region, wherein the VH region comprises the VH of an antibody selected from MSB17, and the VL region comprises the VL of an antibody selected from MBS17.

106. The method of claim 101 or 102, wherein the FVIII epitope is located in the light chain.

107. The method of claim 106, wherein the anti-FVIII antibody or antigen-binding molecule thereof comprises a VH region and a VL region, wherein the VH region comprises the VH of an antibody selected from GMA8013, and the VL region comprises the VL of an antibody selected from GMA8013.

108. The method of claim 101 or 102, wherein the FVIII epitope is located in the A2 domain.

109. The method of claim 108, wherein the anti-FVIII antibody or antigen-binding molecule thereof comprises a VH region and a VL region, wherein the VH region comprises the VH of OBT0037A and the VL region comprises the VL of OBT0037A.

110. The method of any one of claims 101 to 109, wherein the anti-FVIII antibody or antigen-binding molecule thereof binds to the FVIII protein at a dissociation constant (K_{D}) lower than about 1 nM.

111. The method of any one of claims 101 to 110, wherein the anti-FVII antibody or antigen-binding molecule thereof binds to the FVIII protein at a dissociation constant (K_{D}) lower than about 0.1 nM.

112. The method of any one of claims 101 to 111, wherein the anti-FVIII antibody or antigen-binding molecule thereof binds to the FVIII protein at a dissociation constant (K_{D}) lower than about 0.01 nM.

113. The method of any one of claims 101 to 112, wherein the high ionic buffer comprises NaCl, CaCl₂, Tris-HCl, or any combinations thereof.

114. The method of any one of claims 101 to 113, wherein the high ionic buffer comprises at least about 5 nM, at least about 10 nM, at least about 15 nM, at least about 20 nM, at least about 25 nM, at least about 30 nM, at least about 40 nM, at least about 50 nM, at least about 60 nM, at least about 70 nM, at least about 80 nM Tris-HCl.

115. The method of any one of claims 101 to 114, wherein the high ionic buffer comprises at least about 0.1M, at least about 0.2M, at least about 0.3M, at least about 0.4M, at least about 0.5M, at least about 0.6M, at least about 0.7M, at least about 0.8M, at least about 0.9M, at least about 1.0M, at least about 1.1M, at least about 1.2M, at least about 1.3M, at least about 1.4M, or at least about 1.5M NaCl.

116. The method of any one of claims 101 to 115, wherein the high ionic buffer comprises at least about 0.1M CaCl₂, at least about 0.15M, at least about 0.2M, at least about 0.25M, at least about 0.3M, at least about 0.35M, at least about 0.4M, at least about 0.45M, at least about 0.5M, at least about 0.6M, at least about 0.65M, at least about 0.7M, at least about 0.75M, at least about 0.8M, at least about 0.85M, at least about 0.9M, at least about 0.95M, or at least about 1.0M CaCl₂.

117. The method of any one of claims 101 to 116, wherein the high ionic buffer comprises about 20 nM Tris-HCl, about 0.6M NaCl, about 0.35M CaCl₂ at pH 7.2.

118. The method of any one of claims 25 to 117, wherein the FVHI protein comprises FVIII and a heterologous moiety.

119. The method of claim 118, wherein the heterologous moiety comprises a half-life extending moiety.

120. The method of claim 118 or 119, wherein the heterologous moiety comprises a low-complexity polypeptide.

121. The method of claim 118 or 119, wherein the heterologous moiety comprises albumin, albumin binding polypeptide or fatty acid, Fc, transferrin, PAS, the C-terminal peptide (CTP) of the β subunit of human chorionic gonadotropin, polyethylene glycol (PEG), hydroxyethyl starch (HES), albumin-binding small molecules, vWF, a clearance receptor or fragment thereof which blocks binding of the chimeric molecule to a clearance receptor or any combinations thereof.

122. The method of any one of claims 25 to 121, wherein the FVIII protein comprises single chain FVIII or dual chain FVIII.

123. The method of any one of claims 25 to 122, wherein the FVIII protein comprises B-domain deleted FVIII or full-length mature FVIII.

124. The antibody or antigen-binding molecule thereof of any one of claims 3 to 5, comprising the VH of GMA8023 and the VL of GMA8023.

125. The antibody or antigen-binding molecule thereof of any one of claims 3 to 5, comprising the VH of GMA8024 and the VL of GMA8024.

126. The antibody or antigen-binding molecule thereof of any one of claims 3 to 5, comprising the VH of GMA8045 and the VL of GMA8045.

127. The antibody or antigen-binding molecule thereof of any one of claims 3 to 5, comprising the VH of GMA5G8 and the VL of GMA5G8.

128. The antibody or antigen-binding molecule thereof of any one of claims 3 to 5, comprising the VH of GMA8025 and the VL of GMA8025.

129. The antibody or antigen-binding molecule thereof of any one of claims 3 to 5, comprising the VH of GMA8026 and the VL of GMA8026.

130. The antibody or antigen-binding molecule thereof of any one of claims 124 to 129, which is a monoclonal antibody, a chimeric antibody, or a humanized antibody.

131. The antibody or antigen-binding molecule thereof of any one of claims 124 to 129, which is:
(a) a single chain Fv (“scFv”);
(b) a diabody;
(c) a minibody;
(d) a polypeptide chain of an antibody;
(e) F(ab′)₂; or,
(f) F(ab).

132. A nucleic acid molecule or a set of nucleic acid molecules encoding the anti-FVIII antibody or antigen-binding molecule thereof of any one of claims 124 to 129 or a complement thereof.

133. A vector or a set of vectors comprising the nucleic acid molecule of claim 132 or a complement thereof or the set of the nucleic acid molecules of claim 132 or a complement thereof.

134. A host cell comprising the vector of claim 133 or the set of vectors of claim 133.

135. A composition comprising the anti-FVIII antibody or antigen-binding molecule thereof of any one of claims 124 to 131, the nucleic acid molecule or the set of nucleic acid molecules of claim 132, the vector or the set of vectors of claim 133 and a carrier.

136. A kit comprising the anti-FVIII antibody or antigen-binding molecule thereof of any one of claims 124 to 131, the nucleic acid molecule or the set of nucleic acid molecules of claim 132, the vector or the set of vectors of claim 133 and a packaging material.

137. A method for producing an anti-FVIII antibody or antigen-binding molecule thereof comprising culturing the host cell of claim 134 and recovering the anti-FVIII antibody or antigen-binding molecule thereof from the culture medium.

138. The method of claim 55, wherein the anti-FVIII antibody or antigen-binding molecule thereof comprises a VH region and a VL region, wherein the VH region comprises the VH of GMA8045 and the VL region comprises the VL of GMA8045.


SEQUENCE LISTING (3 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 1438 aa, Artificial Sequence
  Mature B-Deleted FVIII construct
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - DNA, 4314 bp, Artificial Sequence
  Mature B-Deleted FVIII construct
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggctagcttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaatcgattatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac